SEQUENCE LISTING (2 sequences)

SEQ ID NO: 1 - RNA, 11 nt, synthetic construct
  source 1..11: mol_type = other RNA
  misc_feature 1..11: note = polyA
aaaaaaaaaaa

SEQ ID NO: 2 - RNA, 13 nt, synthetic construct
  source 1..13: mol_type = other RNA
  misc_feature 1..13: note = polyT
ttttttttttttt

(19) United States
(12) Patent Application Publication
(10) Pub. No.: US 2024/0190957 A1
(43) Pub. Date: Jun. 13, 2024

(54) HIGH-THROUGHPUT METHODS FOR ANALYZING AND AFFINITY-MATURING AN ANTIGEN-BINDING MOLECULE

(71) Applicant: 10x Genomics, Inc., Pleasanton, CA (US)

(72) Inventors: Wyatt James MCDONNELL, Pleasanton, CA (US); Michael John Terry STUBBINGTON, Counterslip Bristol (GB); Katherine PFEIFFER, Pleasanton, CA (US)

(21) Appl. No.: 18/452,127

(22) Filed: Aug. 18, 2023

Related U.S. Application Data
(63) Continuation of application No. PCT/US2022/017067, filed on Feb. 18, 2022.
(60) Provisional application No. 63/151,337, filed on Feb. 19, 2021.
(60) Provisional application No. 63/195,518, filed on Jun. 1, 2021.

(51) Int. Cl. C07K 16/28; C12N 9/22; C12N 9/78; C12N 15/11; C12N 15/85

(52) U.S. Cl. CPC C07K 16/28; C12N 9/22; C12N 9/78; C12N 15/11; C12N 15/85; C07K 2317/622; C12N 2310/20; C12Y 305/04005

(57) ABSTRACT

The present disclosure generally relates to compositions (e.g., nucleic acid constructs, vectors, plasmids, engineered cells, etc.) and methods using the same for selecting and/or making an antigen-binding molecule of interest (e.g., an immune receptor, an antibody or functional fragment thereof) with improved affinity to a target antigen.

### CROSS-REFERENCE TO RELATED APPLICATIONS

This application claims the benefit of U.S. Provisional Patent Application Nos. 63/151,337, filed Feb. 19, 2021, and 63/195,518, filed Jun. 1, 2021, which are incorporated herein by reference in their entireties.

### INCORPORATION OF THE SEQUENCE LISTING

This application contains a Sequence Listing, which is hereby incorporated herein by reference in its entirety. The contents of the electronic sequence listing 2024-01-29 Sequence_Listing_ST26 057862-534C01US.xml; Size: 3,591 bytes; and Date of Creation: Jan. 29, 2024.

### FIELD

The present disclosure generally relates to compositions and methods for analyzing and/or affinity maturing an antigen-binding molecule of interest (e.g., an immune receptor, an antibody or functional fragment thereof). The present disclosure also relates to compositions and methods for engineering an antigen-binding site of an antibody, or antigen-binding fragment thereof, to include an altered characteristic.

### BACKGROUND

Engineering and selection of antibody candidates can yield extremely powerful bio-therapeutics. However, approaches that can narrow down and identify a limited number of particularly potent antibodies are limited in their throughput, and typically require study of individual antibodies expressed by secreted cells. Moreover, it may be desirable to improve affinity of selected lead antibodies, such as via affinity maturation or other optimization processes. However, such techniques also can suffer from low throughput. Because of these limitations, antibody discovery efforts, including lead selection, optimization, and validation, can take months to years. Thus, there is a need for high throughput approaches for identifying potent antibody candidates as well as for affinity maturation. The present disclosure provides a solution to these problems.

### SUMMARY OF THE DISCLOSURE

Provided herein, among others, is a nucleic acid construct. In some embodiments, the nucleic acid construct comprises one or more of the following: a) a first heterologous nucleic acid sequence encoding a self-targeting guide RNA (stgRNA) operably linked to a first promoter, b) a second heterologous nucleic acid sequence encoding a CRISPR-associated (Cas) protein operably linked to a second promoter, wherein the second promoter is induced by an immune response; and c) a third nucleic acid sequence encoding an antigen-binding molecule of interest. In some embodiments, the third nucleic acid sequence is operably linked to a third promoter. In some embodiments, any one of the first, the second, and the third nucleic acid sequence is heterologous or non-heterologous.

In some embodiments, the nucleic acid construct further comprises a fourth nucleic acid sequence encoding a reporter protein. In some embodiments, the fourth nucleic acid sequence is operably linked to a fourth promoter. In some embodiments, the reporter protein is a fluorescent protein.

In certain embodiments, the reporter protein comprises a green fluorescent protein (GFP), eGFP, red fluorescent protein (RFP), BFP, YFP, CFP, citrine, luciferase, GUS, lacZ/beta-galactosidase, chloramphenicol acetyltransferase (CAT), puromycin N-acetyltransferase (PAC), dihydrofolate reductase (DHFR), Sh ble protein, and hygromycin B phosphotransferase.

In some embodiments, one or more of the first, the third, and the fourth promoter independently comprises a strong and/or constitutive promoter. In certain embodiments, the one or more of the strong and/or constitutive promoters are selected from a CMV promoter, an EF1a promoter, a SV39 promoter, a SV40 promoter, a Ubc promoter, a human beta actin promoter, a CAG promoter, a PGK1 promoter, a TRE promoter, a UAS promoter, an Ac5 promoter, a polyhedrin promoter, a CaMKIIa promoter, a GAL 1 promoter, a GAL 10 promoter, a TEF1 promoter, a GDS promoter, a ADH1 promoter, a CaMV35S promoter, a Ubi promoter, a H1 promoter, and a U6 promoter.

In some embodiments, the nucleic acid construct provided herein comprises the first, second, third, and fourth nucleic acid sequences, and the first, the second, and the third nucleic acid sequences are heterologous.

In some embodiments, the Cas protein encoded by the second heterologous nucleic acid sequence comprises a type III, type VI, and type II Cas protein.

In certain embodiments, the Cas protein is selected from the group consisting of Cas9, CasPhi, Cas12, CasRx, Cas13, and Cas3/Cascade protein.

In some embodiments, the stgRNA encoded by the first heterologous nucleic acid sequence comprises one or more protospacer-adjacent motifs (PAMs) configured to target the Cas protein to a sequence of the stgRNA.

In additional embodiments, the nucleic acid construct provided herein further comprises a nucleic acid sequence encoding an enzyme capable of mediating somatic hypermutation (SHM).

In some embodiments, the coding sequence for the SHM enzyme is operably linked to the Cas protein coding sequence via a coding sequence for a linker. In certain embodiments, the linker is a cleavable linker. In exemplary embodiments, the cleavable linker is an autoproteolytic peptide selected from the group consisting of P2A, E2A, F2A, and T2A.

In some embodiments, the coding sequence for the SHM enzyme is operably linked to the Cas protein coding sequence via an internal ribosomal entry site (IRES). In some embodiments, the coding sequence for the SHM enzyme is operably linked to the second promoter. In some exemplary embodiments, the second promoter is selected from the group consisting of a nucleotide factor kappa-B (NFkB) promoter, a MyD88 promoter, an interleukin (IL)-2 promoter, and a JAK/STAT promoter, a Bcl-xL promoter, a c-Rel promoter, a Myc promoter, a NFAT promoter, a mTORC1 promoter, a CD40 promoter, a TLR9 promoter, a Btk promoter, a PI3K promoter, an Arp2/3 promoter, and an IL-4 promoter.

In certain embodiments, the SHM is selected from the group consisting of RAG1, RAG2, APOBEC3F, activation-induced cytidine deaminase (AID), POLH/, Pol η, Pol θ, Polι, Polζ, Pol λ, REV1, PCNA, UNG, and a functional mutant thereof.

Further provided herein is an nucleic acid construct comprising: a) a third nucleic acid sequence encoding an antigen-binding molecule of interest, and b) a nucleic acid sequence encoding an enzyme capable of mediating SHM, wherein the coding sequence for the SHM enzyme is operably linked to an inducible promoter, wherein the inducible promoter is induced by an immune response.

In certain embodiments, the inducible promoter is selected from the group consisting of a nucleotide factor kappa-B (NFkB) promoter, a MyD88 promoter, an interleukin (IL)-2 promoter, and a JAK/STAT promoter, a Bcl-xL promoter, a c-Rel promoter, a Myc promoter, a NFAT promoter, a mTORC1 promoter, a CD40 promoter, a TLR9 promoter, a Btk promoter, a PI3K promoter, an Arp2/3 promoter, and an IL-4 promoter. In other embodiments, the SHM enzyme is selected from the group consisting of RAG1, RAG2, APOBEC3F, activation-induced cytidine deaminase (AID), POLH/, Pol η, Pol θ, Polι, Polζ, Pol λ, REV1, PCNA, UNG, and a functional mutant thereof.

In some embodiments, the third nucleic acid sequence is heterologous or non-heterologous. In some embodiments, the third nucleic acid sequence is operably linked to a promoter. In certain embodiments, the promoter is selected from a CMV promoter, an EF1a promoter, a SV39 promoter, a SV40 promoter, a Ubc promoter, a human beta actin promoter, a CAG promoter, a PGK1 promoter, a TRE promoter, a UAS promoter, an Ac5 promoter, a polyhedrin promoter, a CaMKIIa promoter, a GAL 1 promoter, a GAL 10 promoter, a TEF1 promoter, a GDS promoter, a ADH1 promoter, a CaMV35S promoter, a Ubi promoter, a H1 promoter, and a U6 promoter.

In some embodiments, the antigen-binding molecule comprises an immune receptor, an immunoglobulin, and an antibody or a functional fragment thereof.

In certain embodiments, the antigen-binding molecule comprises an immunoglobulin. In some embodiments, immunoglobulin is selected from the group consisting of IgA, IgD, IgE, IgG, and IgM.

In other embodiments, the antigen-binding molecule comprises an antibody or a functional fragment thereof. In some embodiments, the antibody is a monoclonal antibody or a polyclonal antibody. In certain embodiments, the antibody or a functional fragment thereof comprises a fragment antigen-binding (Fab) fragment, a single-chain variable fragment (scFv), a nanobody, a diabody, a triabody, a minibody, an F(ab′)2 fragment, an F(ab) fragment, a VH domain, a VL domain, a single chain variable fragment (scFv), an Fv fragment, a Fc fragment, a single domain antibody (sdAb), a VNAR domain, and a VHH domain.

In yet other embodiments, the immune receptor comprises a B cell receptor, a chimeric antigen receptor, a membrane-bound antibody, and a membrane-bound immunoglobulin superfamily member. In one exemplary embodiment, the immune receptor is a B cell receptor. In another exemplary embodiment, the immune receptor is a chimeric antigen receptor.

In some embodiments, the third nucleic acid sequence encoding the antigen-binding molecule is configured as a single chain comprising a first segment encoding a first variable region and a second segment encoding a second variable region.

In some embodiments, the third nucleic acid sequence comprises a linker that operably links the first segment and the second segment. In certain embodiments, the linker comprises an autoproteolytic peptide selected from the group consisting of P2A, E2A, F2A, and T2A. In other embodiments, the linker comprises an internal ribosome entry site (IRES). In yet other embodiments, the linker comprises a thrombin cleavage domain. In some embodiments, the coding sequence for the Cas protein is operably linked to a NFkB inducible promoter.

In additional aspects, the present disclosure also encompasses a composition comprising a plurality of the nucleic acid constructs described herein.

In other embodiments, the present disclosure also provides a vector comprising the nucleic acid construct described herein. In some embodiments, the vector further comprises an inducible cellular switch. In some embodiments, the inducible cellular switch comprises rimiducid response element, tetracycline response element, rapamycin response element, ecdysone response element, mifepristone response element, light-based response system, CD24, and HSV-TK/CDK1 pairing.

The present disclosure also provides a composition comprising a plurality of the vectors described herein.

In addition, the present disclosure describes an engineered cell comprising the nucleic acid construct provided herein. In other embodiments, the engineered cell comprises the nucleic acid construct described herein. In some embodiments, the engineered cell is an immune cell, a neuron, an epithelial cell, an endothelial cell, or a stem cell. In one exemplary embodiment, the engineered cell is an immune cell. In some embodiments, the engineered cell expresses the stgRNA, the reporter protein, the Cas protein, and/or the antigen-binding molecule. In some embodiments, the immune cell is a B cell. In certain exemplary embodiments, the engineered cell is a B cell, and wherein the coding sequence for the Cas protein is operably linked to a NFkB inducible promoter. In other embodiments, the immune cell is a T cell.

Further, the present disclosure provides a composition comprising a plurality of the engineered cells described herein.

The present disclosure further provides, among others, a method for analyzing an antigen-binding molecule of interest. In some embodiments, the method for analyzing an antigen-binding molecule of interest comprises a) contacting at least one of the engineered cells of provided herein with a target antigen of the antigen-binding molecule of interest, wherein binding of the target antigen with the antigen-binding molecule expressed by the engineered cells activates an immune response that induces expression of the Cas protein, wherein the induced Cas protein introduces one or more mutations in the nucleic acid sequence of the stgRNA; and b) determining the number of mutations introduced in the nucleic acid sequence of the stgRNA.

In some embodiments, the method for analyzing an antigen-binding molecule of interest further comprises using the determined number of mutations to analyze the binding affinity of the antigen-binding molecule of interest for the antigen. In some embodiments, the determining comprises sequencing all or a part of the nucleic acid sequence encoding the stgRNA expressed by the at least one engineered cell. In some embodiments, the sequencing comprises whole transcriptome sequencing.

In some embodiments, the method for analyzing an antigen-binding molecule of interest further comprises comparing the all or a part of the nucleic acid sequence encoding the stgRNA to the nucleic acid sequence encoding the stgRNA from a reference engineered cell.

In some embodiments, the reference engineered cell: a) expresses an antigen-binding molecule that does not bind to the target antigen; b) does not express an antigen-binding molecule; or c) is an engineered cell whose the coding sequence for Cas protein is not switched on.

In some embodiments, the method for analyzing an antigen-binding molecule of interest further comprises affinity maturing the antigen-binding molecule of interest.

The present disclosure also encompasses a method for affinity maturing an antigen-binding molecule of interest. In some embodiments, the method for affinity maturing an antigen-binding molecule of interest comprises contacting at least one of the engineered cells described herein with a target antigen of the antigen-binding molecule of interest.

In some embodiments, the at least one engineered cell comprises a population of engineered cells. In some embodiments, the method for affinity maturing an antigen-binding molecule of interest further comprises partitioning the population of engineered cells into partitions of a plurality of partitions such that a partition of the plurality of partitions comprises a single engineered cell. In some embodiments, the plurality of partitions comprise a plurality of microwells or a plurality of droplets. In some embodiments, the partitioning occurs prior to or after the contacting. In some embodiments, the partition of the plurality of partitions further comprises a plurality of nucleic acid barcode molecules comprising a partition-specific barcode sequence.

In additional embodiments, the method for affinity maturing an antigen-binding molecule of interest further comprises using a nucleic acid barcode molecule of the plurality of nucleic acid barcode molecules and an analyte of the engineered cell to generate a barcoded analyte of the engineered cell. In some embodiments, at least a subset of the plurality of nucleic acid barcode molecules are releasably attached to a gel bead.

In certain embodiments, the method for affinity maturing an antigen-binding molecule of interest further comprises releasing the nucleic acid barcode molecules from the gel bead prior to generating a first barcoded nucleic acid molecule. In some embodiments, the gel bead is degradable. In certain embodiments, the nucleic acid barcode molecules are released from the gel bead through degradation of the gel bead. In other embodiments, the nucleic acid barcode molecules are released from the gel bead through cleavage of a linkage between the nucleic acid barcode molecule and the gel bead.

In some embodiments, the nucleic acid barcode molecule further comprises a unique molecular identifier (UMI) sequence.

In some embodiments, the UMI sequence of the nucleic acid barcode molecule differs from the UMI sequence of another nucleic acid barcode molecule of the plurality of nucleic acid barcode molecules in the partition.

In certain embodiments, the nucleic acid barcode molecule further comprises a functional sequence. In other embodiments, nucleic acid barcode molecule further comprises a capture sequence.

In some embodiments, any one or more of the first nucleic acid sequence, the second nucleic acid sequence, and third nucleic acid sequence further comprises a capture handle sequence that is complementary to the capture sequence of the nucleic acid barcode molecule.

In certain embodiments, the capture sequence comprises a template switch oligonucleotide (TSO) sequence. In other embodiments, the capture sequence comprises a polyT sequence. In yet other embodiments, the capture sequence comprises a reporter capture sequence.

In some embodiments, the target antigen is coupled to a reporter oligonucleotide, wherein the reporter oligonucleotide comprises a reporter barcode sequence. In certain embodiments, the reporter oligonucleotide further comprises a reporter capture handle and/or a functional sequence. In other embodiments, the reporter capture handle comprises a sequence that is complementary to the reporter capture sequence.

In some embodiments, the method for affinity maturing an antigen-binding molecule of interest further comprises identifying a single engineered cell of the population of the engineered cells of which the number of mutations in the mutated stgRNA is higher than the mutation number determined in the stgRNA of the reference engineered cell.

Provided herein, among others, also includes a method of making an antigen-binding molecule of interest. In some embodiments, the method of making an antigen-binding molecule of interest comprises a) identifying the antigen-binding molecule of interest by method for analyzing an antigen-binding molecule of interest described herein, and b) purifying and/or producing the antigen-binding molecule from the identified single engineered cell expressing the antigen-binding molecule thereof. In other aspects, the present disclosure further provides an antigen-binding molecule identified by the method for analyzing an antigen-binding molecule of interest described herein.

In another aspect, the disclosure provides for a method of engineering an antigen-binding site of an antibody, or antigen-binding fragment thereof, to have an altered characteristic. In the method, a nucleic acid sequence encoding a selected antibody, or selected antigen-binding fragment thereof, is provided. The selected antibody, or selected antigen-binding fragment thereof, binds a target antigen. The nucleic acid sequence is amplified in an error-prone amplification reaction to produce a plurality of polynucleotides encoding variant antibodies, or variant antigen-binding fragments thereof. The plurality of variant antibodies, or variant antigen-binding fragments thereof, is expressed in a plurality of engineered cells. The plurality of engineered cells have a nucleic acid construct that includes one or more of (a) a first heterologous nucleic acid sequence encoding a stgRNA operably linked to a first promoter, (b) a second heterologous nucleic acid sequence encoding a CRISPR-associated (Cas) protein operably linked to a second promoter, wherein the second promoter is induced by an immune response; and (c) a third nucleic acid sequence encoding an antigen-binding molecule of interest, wherein the antigen-binding molecule of interest is the variant antibody, or variant antigen-binding fragment thereof. An engineered cell of the plurality of engineered cells expresses the variant antibody, or variant antigen binding fragment thereof, of the plurality of variant antibodies, or variant antigen binding fragments thereof. The engineered cell of the plurality of engineered cells is contacted with the target antigen. Binding the target antigen with the variant antibody, or variant antigen binding fragment thereof, activates an immune response that induces expression of the Cas protein. The inducing expression of the Cas protein introduces one or more mutations in the nucleic acid sequence of the stgRNA. The number of mutations introduced in the nucleic acid sequence of the stgRNA is determined. The variant antibody, or variant antigen-binding fragment thereof, is identified to comprise the altered characteristic based on the determined number of mutations.

In yet another aspect, the disclosure provides for a method of preparing a library of variant antibodies, or variant antigen-binding fragments thereof, comprising an altered characteristic or characteristics. In the method, a nucleic acid sequence encoding a selected antibody, or selected antigen-binding fragment thereof, is provided. The selected antibody, or selected antigen-binding fragment thereof, binds a target antigen. The nucleic acid sequence is amplified in an error-prone amplification reaction to produce a plurality of polynucleotides encoding variant antibodies, or variant antigen-binding fragments thereof. The plurality of variant antibodies, or variant antigen-binding fragments thereof, is expressed in a plurality of engineered cells. The plurality of engineered cells have a nucleic acid construct that includes one or more of (a) a first heterologous nucleic acid sequence encoding a stgRNA operably linked to a first promoter, (b) a second heterologous nucleic acid sequence encoding a CRISPR-associated (Cas) protein operably linked to a second promoter, wherein the second promoter is induced by an immune response; and (c) a third nucleic acid sequence encoding an antigen-binding molecule of interest, wherein the antigen-binding molecule of interest is the variant antibody, or variant antigen-binding fragment thereof. An engineered cell of the plurality of engineered cells expresses the variant antibody, or variant antigen binding fragment thereof, of the plurality of variant antibodies, or variant antigen binding fragments thereof. The engineered cell of the plurality of engineered cells is contacted with the target antigen. Binding the target antigen with the variant antibody, or variant antigen binding fragment thereof, activates an immune response that induces expression of the Cas protein. The inducing the expression of the Cas protein introduces one or more mutations in the nucleic acid sequence of the stgRNA. The number of mutations introduced in the nucleic acid sequence of the stgRNA is determined. The variant antibody, or variant antigen-binding fragment thereof, is identified for inclusion in the library based on the determined number of mutations.

In some embodiments, the provided nucleic acid sequence encoding the selected antibody, or selected antigen-binding fragment thereof, is of a plurality of nucleic acid sequences encoding a plurality of selected antibodies, or selected antigen-binding fragments thereof, that bind the target antigen.

In some embodiments, the selected antibody, or selected antigen-binding fragment thereof, comprises a human antibody, or antigen-binding fragment thereof.

In some embodiments, the selected antibody, or selected antigen-binding fragment thereof, comprises a human antibody.

In certain embodiments, the provided nucleic acid sequence encoding the selected antibody, or selected antigen-binding fragment thereof, is derived from nucleic acids of a cell of a human donor. In some embodiments, the cell is from a blood sample or a peripheral blood mononuclear cell sample of the human donor. In some embodiments, the cell is a B cell.

In some embodiments, the plurality of nucleic acid sequences encoding the plurality of selected antibodies, or selected antigen-binding fragments thereof, are derived from nucleic acids of a combination of cells from multiple human donors. In some embodiments, the cells from the multiple human donors are from blood samples and/or peripheral blood mononuclear cell samples of the human donors. In some embodiments, the cells are B cells.

In some embodiments, the donor has been exposed to the target antigen, is suspected of having been exposed to the target antigen, or is suspected of being resistant to the target antigen.

In certain embodiments, the donors have been exposed to the target antigen, or are been suspected of having been exposed to the target antigen, or are suspected of being resistant to the target antigen.

In some embodiments, the provided nucleic acid sequence encoding the selected antibody, or selected antigen-binding fragment thereof, is derived from nucleic acids of a cell of mouse. In some embodiments, the mouse is a transgenic mouse. In some embodiments, the transgenic mouse expresses human immunoglobulin genes. In some embodiments, the cell of the mouse is from a blood sample of the mouse. In some embodiments, the cell is a B cell. In some embodiments, the mouse had been exposed to the target antigen.

In some embodiments, the plurality of nucleic acid sequences encoding the plurality of selected antibodies, or selected antigen-binding fragments thereof, are derived from nucleic acids of a combination of cells from mice. In some embodiments, the cells from the mice are from blood samples of the mice. In some embodiments, the cells are B cells. In some embodiments, the mice had been exposed to the target antigen.

In certain embodiments, the altered characteristic comprises an altered affinity for the target antigen, based on the determined number of mutations introduced in the nucleic acid sequence of the stgRNA. In some embodiments, the method further comprises sequencing all or a part of the nucleic acid sequence encoding the stgRNA to determine the number of mutations introduced in the nucleic acid sequence of the stgRNA. In some embodiments, the sequencing comprises whole transcriptome sequencing. In some embodiments, the number of mutations is determined by comparing the all or a part of the nucleic acid sequence encoding the stgRNA to the nucleic acid sequence encoding the stgRNA from a reference engineered cell. In some embodiments the reference engineered cell (a) expresses the selected antibody, or selected antigen-binding fragment thereof as the antigen-binding molecule, (b) expresses an antigen-binding molecule that does not bind to the target antigen; (c) does not express an antigen-binding molecule; or (d) is an engineered cell whose the coding sequence for Cas protein is not switched on.

In some embodiments, the method further comprises partitioning the plurality of engineered cells into partitions of a plurality of partitions such that a partition of the plurality of partitions comprises a single engineered cell. In some embodiments, the partitioning occurs prior to, or after, the contacting. In some embodiments, the partition of the plurality of partitions further comprises a plurality of nucleic acid barcode molecules comprising a partition-specific barcode sequence. In some embodiments, the method further comprises generating a plurality of barcoded nucleic acid molecules comprising a nucleic acid sequence of an analyte of the engineered cell or reverse complement thereof and the partition-specific barcode sequence or reverse complement thereof.

In other embodiments, the plurality of nucleic acid barcode molecules further comprise a capture sequence.

In certain embodiments, the analyte is an mRNA or DNA analyte and the capture sequence is configured to couple to the mRNA or DNA analyte. In some embodiments, the capture sequence couples to the mRNA or DNA analyte by complementary base pairing. In some embodiments, the analyte is the mRNA analyte and capture sequence configured to couple to the mRNA analyte comprises a polyT sequence.

In certain embodiments, the analyte is a complementary DNA (cDNA) analyte reversed transcribed from a cell mRNA and the capture sequence is configured to couple to non-templated nucleotides appended to the cDNA during reverse transcription of the mRNA. In some embodiments, the mRNA is reverse transcribed into cDNA utilizing a primer comprising a polyT sequence. In some embodiments, the non-templated nucleotides comprise a cytosine. In some embodiments, the capture sequence configured to couple to the cDNA comprise a guanine. In some embodiments, coupling of the capture sequence to the non-templated cytosine permits reverse transcription of the cDNA to extend into the nucleic acid barcode molecule.

In certain embodiments, the capture sequence comprises a template switch oligonucleotide sequence.

In some embodiments, the plurality of nucleic acid barcode molecules further comprises a functional sequence.

In some embodiments, the analyte is derived from any one or more of the first nucleic acid sequence, the second nucleic acid sequence, and the third nucleic acid sequence. In some embodiments, the method further comprises determining sequence of a barcoded nucleic acid molecule of the barcoded nucleic acid molecules.

In certain embodiments, the altered affinity is a higher affinity. In some embodiments, the altered affinity is due to an alteration in association constant. In other embodiments, the altered affinity is due to an alteration in dissociation constant. In other embodiments, the altered affinity is due to an alteration in specificity for the target antigen.

In some embodiments, the method further comprises subjecting the identified variant antibody, or identified variant antigen-binding fragment thereof, to an activity assay. In some embodiments, the identified variant antibody, or identified variant antigen-binding fragment thereof, further comprises an altered activity.

In certain embodiments, wherein the target antigen is a pathogen.

In some embodiments, the target antigen is a virus, a bacterial cell or a parasite.

In some embodiments, the target antigen is a virus-like particle or lipoparticle.

In some embodiments, the target antigen is a cytokine or a fragment thereof.

In some embodiments, the target antigen is a tumor-associated antigen or a fragment thereof.

In some embodiments, the tumor-associated antigen is a growth factor, a growth factor receptor, or a fragment thereof.

In some embodiments, the target antigen is an autoantigen or a fragment thereof.

In certain embodiments, the error-prone amplification comprises rolling circle amplification.

In certain embodiments, the error-prone amplification introduces errors via a low fidelity polymerase enzyme.

In some embodiments, the error-prone amplification introduces errors via increasing MgCl₂ concentration, addition of MnCl₂, or both increasing MgCl₂ concentration and addition of MnCl₂ in the reaction.

In some embodiments, the error-prone amplification introduces errors via an inbalance in ratio in deoxynucleoside triphosphate concentrations in the reaction.

In certain embodiments, the variant antibody, or variant antigen-binding fragment thereof, comprises at least one amino acid substitution relative to the selected antibody or antigen binding fragment thereof.

In some embodiments, the variant antibody, or variant antigen-binding fragment thereof, comprises at least three amino acid substitutions relative to the selected antibody or antigen binding fragment thereof.

In some embodiments, the variant antibody, or variant antigen-binding fragment thereof, comprises at least five amino acid substitutions relative to the selected antibody or antigen binding fragment thereof.

The foregoing is merely a summary and is illustrative only. It is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 shows an exemplary microfluidic channel structure for partitioning individual biological particles in accordance with some embodiments of the disclosure.

FIG. 2 shows an exemplary microfluidic channel structure for the controlled partitioning of beads into discrete droplets.

FIG. 3 shows an exemplary barcode carrying bead.

FIG. 4 illustrates another example of a barcode carrying bead.

FIG. 5 schematically illustrates an example microwell array.

FIG. 6 schematically illustrates an example workflow for processing nucleic acid molecules.

FIG. 7 schematically illustrates examples of labelling agents.

FIG. 8 depicts an example of a barcode carrying bead.

FIGS. 9A-9C schematically depict an example workflow for processing nucleic acid molecules.

FIGS. 10A-10C schematically illustrate exemplary constructs of the present disclosure.

FIG. 11 shows an example of a microfluidic channel structure for delivering barcode carrying beads to droplets.

FIG. 12 illustrates a workflow for the enrichment of a nucleic acid sequence of interest.

FIG. 13 illustrates a nested PCR scheme for amplification of a nucleic acid sequence of interest.

FIG. 14 provides a pictorial outline of a nucleic acid sequence that is compatible with a vector, including incorporation of the nucleic acid sequence into the vector.

FIG. 15 provides a pictorial outline of an exemplary probe and scheme for capture-based enrichment of nucleic acid sequences of interest.

FIG. 16 provides a pictorial outline for a method of enriching a nucleic acid sequence of interest.

### DETAILED DESCRIPTION OF THE DISCLOSURE

The present disclosure relates to nucleic acid constructs (e.g., nucleic acid constructs comprising one or more nucleic acid sequences) and methods of use thereof. The nucleic acid constructs provided herein are useful in identification of antigen-binding molecules of interest that recognize a target antigen with particularly high affinity. In some embodiments, the nucleic acid constructs and methods provided herein can detect the affinity maturation of antigen-binding molecules over time. Thus, one aspect of the disclosure relates to nucleic acid constructs comprising nucleic acid sequences that encode RNA and/or protein. In some embodiments, the constructs can be configured as expression cassettes or vectors containing nucleic acid sequences operably linked to other heterologous nucleic acid sequences such as, for example, regulatory sequences that allow in vivo expression of the RNA or protein in a host cell.

The present disclosure further relates to methods. The methods include those useful for selecting and/or making an antigen-binding molecule of interest. For instance, the methods for selecting an antigen-binding molecule of interest utilize the constructs provided herein to identity antigen-binding molecules that higher affinity with a target antigen. In some embodiments, the identified antigen-binding molecule has higher affinity with a target antigen due to the mutations introduced in the antigen-binding molecule by the constructs provided herein.

In other methods, an antigen-binding site of an antibody, or antigen-binding fragment thereof, may be engineered to include an altered characteristic. In yet other methods, a library of variant antibodies, or variant antigen-binding fragments thereof, having an altered characteristic or characteristics may be prepared. In these methods, a nucleotide sequence of a selected antibody, or selected antigen-binding fragment thereof is provided and subject to error-prone amplification to produce polynucleotides encoding variant antibodies, or variant antigen-binding fragments thereof. Expressing the variant antibodies, or variant antigen-binding fragments thereof, in cells that comprise the nucleic acid sequences encoding the nucleic acid constructs provided herein, are useful to identifying those of the variant antibodies, or variant antigen-binding fragments thereof, having the altered characteristic (or for inclusion in the library). Such variant antibodies or antigen-binding fragments thereof may be expressed in cells via one or more of the constructs provided herein.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

An “adapter,” an “adaptor,” and a “tag” are terms that are used interchangeably in this disclosure, and refer to moieties that can be coupled to a polynucleotide sequence (in a process referred to as “tagging”) using any one of many different techniques including (but not limited to) ligation, hybridization, and tagmentation. Adapters can also be nucleic acid sequences that add a function, e.g., spacer sequences, primer sequences, primer binding sites, barcode sequences, and unique molecular identifier sequences.

The term “barcode” is used herein to refer to a label, or identifier, that conveys or is capable of conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

“Cancer” refers to the presence of cells possessing several characteristics typical of cancer-causing cells, such as uncontrolled proliferation, immortality, metastatic potential, rapid growth and proliferation rate, and certain characteristic morphological features. Some types of cancer cells can aggregate into a mass, such as a tumor, but some cancer cells can exist alone within a subject. A tumor can be a solid tumor, a soft tissue tumor, or a metastatic lesion. As used herein, the term “cancer” also encompasses other types of non-tumor cancers. Non-limiting examples include blood cancers or hematological malignancies, such as leukemia, lymphoma, and myeloma. Cancer can include premalignant, as well as malignant cancers.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

As used herein, the term “functional fragment thereof” or “functional variant thereof” relates to a molecule having qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. When referring to a Cas protein, the skilled artisan in the art will understand that a functional variant of a wild-type protein (e.g., a wild-type Cas protein) can include a peptide that has a substantial activity of the wild-type protein, e.g. at least about 30%, about 40%, about 50%, about 60%, about 70%, about 80%, about 90%, about 95% or about 100% of the activity that the wild-type protein exhibits. In some embodiments, one having ordinary skill in the art can use a number of methods known in the field to test the functionality or activity of a compound, e.g. peptide or protein. In some embodiments, the functional variant of the encoded wild-type protein can also include any fragment of the wild-type protein or fragment of a modified protein that has conservative modification on one or more of amino acid residues in the corresponding full length, wild-type protein. In some embodiments, the functional variant of the encoded wild-type protein can also include any modification(s), e.g. deletion, insertion and/or mutation of one or more amino acids that do not substantially negatively affect the functionality of the wild-type protein.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the orthogonal DNA target sequences described herein or the promoter sequence in a nucleic acid construct, or in an engineered response element means that the orthogonal DNA target sequences and the promoters are in-frame and in proper spatial and distance away from a polynucleotide of interest coding for a protein or an RNA to permit the effects of the respective binding by transcription factors or RNA polymerase on transcription.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

The term “biological particle” is used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle, e.g., a nucleus. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ± up to 10%, up to ±5%, or up to ±1%.

The term “microwell,” as used herein, generally refers to a well with a volume of less than 1 mL. Microwells may be made in various volumes, depending on the application. For example, microwells may be made in a size appropriate to accommodate any of the partition volumes described herein.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Compositions

### A. Nucleic Add Constructs

In some embodiments, the present disclosure provides nucleic acid constructs comprising one or more nucleic acid sequences. In one aspect, the nucleic acid constructs provided herein are useful in fast and high throughput identification of antigen-binding molecules of interest that recognize an antigen with particularly high affinity. In some embodiments, the nucleic acid constructs and methods provided herein can detect the affinity maturation of antigen-binding molecules over time. In other embodiments, the nucleic acid constructs and methods provided herein can identify antibodies, or antigen-binding fragments thereof, that have been engineered to have an altered characteristic, or to include in a library of antibodies having one or more altered characteristics.

For instance, in some embodiments, the nucleic acid constructs provided herein comprise one or more of: a first nucleic acid sequence encoding a self-targeting guide RNA (stgRNA), a second nucleic acid sequence encoding a CRISPR-associated (Cas) protein, and/or a third nucleic acid sequence encoding an antigen-binding molecule of interest. In other embodiments, the present disclosure provides nucleic acid constructs comprising a nucleic acid sequence encoding an antigen-binding molecule of interest, and a nucleic acid sequence encoding an enzyme capable of mediating somatic hypermutation (SHM).

Thus, one aspect of the disclosure relates to nucleic acid sequences that encode an RNA or protein. In some embodiments, each of the nucleic acid sequences of the disclosure can be configured as expression cassettes or vectors containing these nucleic acid sequences operably linked to heterologous nucleic acid sequences such as, for example, regulatory sequences that allow in vivo expression of the RNA or protein in a host cell.

The nucleic acid sequences provided herein can contain naturally occurring sequences, or sequences that differ from those that occur naturally, but, due to the degeneracy of the genetic code, encode the same polypeptide, e.g., antibody. These nucleic acid sequences can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid sequences can be double-stranded or single-stranded (e.g., either a sense or an antisense strand).

The nucleic acid sequences are not limited to sequences that encode RNAs (e.g., stgRNA) or proteins. Some or all of the non-coding sequences that lie upstream or downstream from a coding sequence (e.g., the coding sequence of an antibody) can also be included. Those of ordinary skill in the art of molecular biology are familiar with routine procedures for isolating nucleic acid sequences.

Nucleic acid sequences of the present disclosure can be of any length, including for example, between about 50 base pairs (bp) and about 100 bp, about 75 bp and about 150 bp, about 100 bp and about 200 bp, about 150 bp and about 250 bp, about 200 bp and about 300 bp, about 250 bp and about 350 bp, about 300 bp and about 400 bp, about 350 bp and about 450 bp, about 400 bp and about 500 bp, about 450 bp and about 550 bp, about 500 bp and about 1 Kb, about 1 Kb and about 5 Kb, about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the nucleic acid construct of the present disclosure comprises a nucleic acid sequence encoding a stgRNA. In some embodiments, the nucleic acid sequence encoding a stgRNA is referred to as the first nucleic acid sequence. In some embodiments, the stgRNA comprises one or more protospacer-adjacent motifs (PAMs). In certain embodiments, the one or more PAMs are configured to target the Cas protein to a sequence of the stgRNA. In some embodiments, the stgRNA is operably linked to a promoter (e.g., the first promoter). In certain embodiments, the stgRNA is constitutively expressed through a CMV promoter or similar strong promoters. Promoters encompassed by the present disclosure are described in detail below.

Generally, the RNA-guided DNA endonucleases, such as the Cas proteins as described herein, introduce a break in the target DNA, either double-stranded or single-stranded, containing one or more PAMs and homology to the specificity determining sequence of a small guide RNA. In certain embodiments, the PAM sequence is located 3′ of the specificity determining sequence in the small guide RNA. Once a break is introduced, the targeted DNA can be repaired via error-prone DNA repair mechanisms in cells, e.g., human cells. In some embodiments, the small guide comprising a PAM sequence acts as a stgRNA that directs the RNA-guided DNA endonuclease to cleave its own encoding DNA, generating mutagenized stgRNA locus. Consequently, in some embodiments, the mutagenized stgRNA locus would continue to be transcribed and enact additional rounds of continuous, self-targeted mutagenesis. Thus, in some embodiments, the stgRNA locus acquires mutations corresponding to the level of activity of the Cas-stgRNA (e.g., Cas9-stgRNA) complex. In certain embodiments, sequencing of the stgRNA itself and the mutations obtained by the stgRNA can be traced to progeny cells of the founder host cells by performing lineage analysis of the stgRNA and the antigen-binding molecule of interest, as described herein.

In some embodiments, the Cas protein recognizes a “protospacer-adjacent motif” (PAM). The term PAM is used herein as its conventional meaning in the field. A PAM generally is a 2-6 base pair DNA sequence immediately following the DNA sequence targeted by a Cas nuclease, e.g., the Cas9 nuclease, in the CRISPR bacterial adaptive immune system. PAM is an essential targeting component which distinguishes bacterial self from non-self DNA, thereby preventing the CRISPR locus from being targeted and destroyed by the CRISPR-associated nuclease. In some embodiments, the PAM includes the canonical PAM sequence 5′-NGG-3′ or 5′-NNGG-3′, where “N” is any nucleobase followed by two guanine (“G”) nucleobases. However, many Cas nucleases and PAMs are also suitable for the compositions and methods of the present disclosure. Thus, other PAM sequences that have been identified or to be identified are also encompassed by the present disclosure.

The stgRNA sequence of the present disclosure can be up to 200 bp in length. In some embodiments, the stgRNA is about 5 to about 200 bp. In other embodiments, the stgRNA is about 10 to about 100 bp. In some embodiments, the stgRNA is about 15 to about 60 bp. In certain embodiments, the stgRNA is about 20 to about 40 bp.

In some embodiments, the nucleic acid construct of the present disclosure comprises a nucleic acid sequence encoding a Cas protein or a functional variant thereof. In some embodiments, the nucleic acid sequence encoding a Cas protein is referred to as the second nucleic acid sequence. CRISPR-associated (Cas) proteins are generally known in the art. The clustered regularly interspaced short palindromic repeats (CRISPR)-Cas (CRISPR-associated proteins) is a prokaryotic adaptive immune system that is represented in most archaea and many bacteria. Numerous Cas proteins have been characterized and mainly comprise nucleases, helicases, or RecB-family exonucleases. The CRISPR-Cas system is generally classified into two classes and six types (Type I to Type VI), each of which has several subtypes. The distinction between the CRISPR-Cas types is based on the respective signature genes and the typical organization of the respective loci. While CRISPR systems of types I, III and IV (Class 1) involve multiple Cas proteins to degrade DNA/RNA, Type II, V and VI (Class 2) require single multifunctional proteins to achieve nucleic acid degradation. Generally, there are no particular limitations regarding the Cas proteins suitable for the compositions and methods described herein. In some embodiments, the Cas protein comprises a type III, type VI, or type II Cas protein. In certain embodiments, the Cas protein comprises a type II Cas protein. In an exemplary embodiment, the Type II Cas protein comprises naturally occurring Cas9 proteins, orthologs, and engineered variants thereof. However, other Cas nuclease systems can also be adapted to the nucleic acid constructs and methods disclosed in the present disclosure. The guide RNA for each Cas protein may vary. A skilled person in the art would understand how to choose and/or design the nucleic acid constructs disclosed herein to be used with different Cas systems. Exemplary Cas proteins that can be used with the nucleic acid constructs and methods of the present disclosure include, without limitation, CasPhi, Cas12, CasRx, Cas13, and Cas3/Cascade protein, and orthologs and engineered variants thereof.

In certain embodiments, the CRISPR-Cas-based nucleic acid constructs can be used as molecular recorders by tracking cellular responses in the form of nucleic acid sequence alterations. For instance, in some embodiments, the stgRNAs is generated so that expression of Cas9 and the stgRNA will result in cleavage and insertion/deletion (indel) mutation accumulation at the stgRNA loci. Thus, a cellular response (e.g., the binding of an antigen-binding molecule to a target antigen) can be “recorded” by linking cellular responses with the expression of the stgRNA or Cas9. In some embodiments, by sequencing the stgRNA locus and determining the level of accumulated mutations, the duration or intensity of the stimulus can be measured. Alternatively, cellular activity can be recorded as individual nucleic acid sequence alterations using single-base editors targeted to designated positions on the endogenous genomic DNA of an engineered cell described herein.

In some embodiments, Cas9-mediated nucleic acid sequence alterations are inherited from the founder cell to its descendants (e.g., progeny cells), and therefore mutations can be used for cell-lineage tracing. To perform whole-organism lineage tracing, accumulation of mutations over multiple rounds of cell division can be recorded following the delivery of the nucleic acid construct comprising Cas9 and stgRNA into an engineered cell. By tracking these mutations in hundreds of thousands of progeny cells from individual founder cell, a cellular event (e.g., the binding of an antigen-binding molecule to a target antigen) can be traced.

Alternative recording strategies using different Cas systems are also known in the art and encompassed by the present disclosure. As a non-limiting example, another recording strategy can be based on integrating nucleotides into bacterial genomic crRNA arrays as traceable molecular events. This strategy utilizes the natural adaptation process of prokaryotic CRISPR-Cas systems, in which appropriate Cas proteins (e.g., CasI and Cas2 proteins) capture short fragments of invading plasmid or phage genetic material and integrate the exogenous sequences as spacers into a crRNA array.

The nucleic acid sequence encoding the Cas protein can be operably linked to a promoter (e.g., the second promoter). In some embodiments, the second promoter is inducible by an immune response. For instance, the second promoter can be induced by recognition of a target antigen as described herein by the cell. In other embodiments, the second promoter can be induced by the binding of the cell to the target antigen. In some embodiments, the second promoter is induced by a transcription factor that is activated by antigen-mediated immune receptor stimulation. Exemplary promoters inducible by an immune response, e.g., by antigen-mediated immune receptor stimulation, include, but are not limited to, a nucleotide factor kappa-B (NFkB) promoter (i.e. an NFkB-inducible promoter), a MyD88 promoter, an interleukin (IL)-2 promoter, and a JAK/STAT promoter, a Bcl-xL promoter, a c-Rel promoter, a Myc promoter, a NFAT promoter, a mTORC1 promoter, a CD40 promoter, a TLR9 promoter, a Btk promoter, a PI3K promoter, an Arp2/3 promoter, and an IL-4 promoter. In certain embodiments, the coding sequence for the Cas protein is operably linked to a NFkB inducible promoter.

In some embodiments, the nucleic acid construct of the present disclosure comprises an immune responsive enhancer. Immune responsive enhancers are generally known in the art. For instance, non-limiting examples of the immune responsive enhancer suitable for the compositions and methods described herein can include transcription factor (TF)-responsive enhancer, interleukin 2 receptor alpha (IL2RA) enhancer, nuclear factor of activated T-cells (NFAT), c-Myc, Jun, activating transcription factor 2 (ATF-2), MYC associated factor X (MAX), interferon regulatory factor 4 (IRF4), IRF8, B lymphocyte-induced maturation protein-1 (Blimp-1), transcription factor 3 (TCF3), TCF4, signal transducer and activator of transcription 3 (STAT3), STAT6, and inhibitor of DNA binding 3 (ID3).

In some exemplary embodiments, engineered cells of the present disclosure that bind the target antigen trigger an immune response. Such immune response can activate the inducible promoter and drive expression of Cas, thereby enabling the stgRNA to target itself and induce mutations in the guide RNA (e.g., up to 200 bp long). In some embodiments, the number of mutations in the stgRNA is used to quantify antigen binding. In some embodiments, the number of mutations acquired relative to reference cells can be used to quantify a fold-change or similar change in induced NFkB activity and/or to compensate for background NFkB activity induced by the experimental conditions.

In other exemplary embodiments, sequencing of the stgRNA itself and the mutations obtained by the stgRNA can be traced to progeny cells of the founder host cells (e.g., the original clone) by performing lineage analysis of the stgRNA and the antigen-binding molecule of interest, as described above.

In some embodiments, the nucleic acid constructs provided herein further comprises a nucleic acid sequence encoding an enzyme capable of mediating somatic hypermutation (SHM). In some embodiments, the coding sequence for the SHM enzyme is operably linked to a promoter (e.g., the second promoter), wherein the promoter is inducible by an immune response. Exemplary promoters are described herein. Without being bound by theory, a cellular immune response, e.g., by antigen-mediated immune receptor activation, can trigger the expression of the SHM enzyme. The SHM enzyme can promote hypermutation of variable region genes encoding an antigen-binding molecule, hence promoting somatic hypermutation of the antigen-binding molecule and affinity maturation.

The term “affinity maturation” generally refers to a process involving accumulation of mutations in variable genes encoding antigen-binding molecules and selection of variants with high antigen affinity. As described herein, the affinity maturation may occur in vitro or in vivo. Thus, in some embodiments, the construct enables somatic hypermutation to be selectively driven by antigen-binding and enables the accumulation of additional mutations that result in enhanced binding affinity relative to the originally isolated clone.

SHM enzymes are generally known in the art. Non-limiting, exemplary suitable SHM enzymes include RAG1, RAG2, APOBEC3F, activation-induced cytidine deaminase (AID), POLH/, Pol η, Pol θ, Polι, Pol ζ, Pol λ, REV1, PCNA, and UNG. In some embodiments, the SHM enzyme encompassed by the present disclosure comprises a functional mutant of any suitable SHM enzymes.

In some embodiments, the SHM enzyme coding sequence is operably linked to the Cas protein coding sequence. In some embodiments, the SHM enzyme coding sequence is operably linked to the Cas protein coding sequence via a linker as described herein. In some embodiments, the linker can be a cleavable linker. In certain embodiments, the Cas protein coding sequence is fused to the coding sequence for an SHM enzyme (e.g., Rag or AID) via a self-cleaving linker. Exemplary self-cleaving linkers include, but are not limited to P2A, E2A, F2A, and T2A. In some embodiments, the SHM enzyme coding sequence is operably linked to the Cas protein coding sequence by an internal ribosome entry site (IRES). In other embodiments, the SHM enzyme coding sequence is operably linked to the Cas protein coding sequence by a thrombin cleavage domain.

Further, the coding sequence for the SHM enzyme can be operably linked to the same promoter as the one operably linked to the nucleic acid sequence encoding the Cas protein (i.e., the second promoter). Promoters encompassed by the present disclosure are described in detail below. Non-limiting, exemplary suitable second promoters include a nucleotide factor kappa-B (NFkB) promoter, a MyD88 promoter, an interleukin (IL)-2 promoter, and a JAK/STAT promoter, a Bcl-xL promoter, a c-Rel promoter, a Myc promoter, a NFAT promoter, a mTORC1 promoter, a CD40 promoter, a TLR9 promoter, a Btk promoter, a PI3K promoter, an actin-related protein 2/3 (Arp2/3) promoter, and an IL-4 promoter. In some exemplary embodiments, the Arp2/3 promoter is used where antigen-presenting cells are used to activate the B cells with constructs provided herein. In other exemplary embodiments, an IL-4 promoter can be used where cells are stimulated with IL-4 to induce cytidine deaminase (AID) activity.

In some embodiments, the nucleic acid construct of the present disclosure comprises a nucleic acid sequence encoding an antigen-binding molecule of interest. In some embodiments, the nucleic acid sequence encoding an antigen-binding molecule of interest is referred to as the third nucleic acid sequence. In one exemplary embodiment, the nucleic acid sequence encoding an antigen-binding molecule of interest is heterologous to the cells (e.g., the recombinant or engineered cells described herein). In some embodiments, the nucleic acid sequence encoding an antigen-binding molecule of interest is operably linked to a promoter. In certain embodiments, the nucleic acid sequence encoding an antigen-binding molecule of interest is operably linked to a constitutive promoter. In some embodiments, the promoter linked to the nucleic acid sequence encoding an antigen-binding molecule of interest is referred to as the third promoter.

In some embodiments, the antigen-binding molecule comprises an immune receptor. An immune receptor as used herein generally refers to a cell surface molecule on cells of the immune system that specifically binds surface molecules or messenger molecules. Most of immune receptors were first identified in the immune system.

In certain exemplary embodiments, the immune receptors encompassed by the present disclosure comprise B cell receptors, chimeric antigen receptors, membrane-bound antibodies, and membrane-bound immunoglobulin superfamily members.

In some embodiments, the immune receptor comprises an antigen-specific receptor, e.g., a receptor that can immunologically recognize and/or specifically bind to an antigen, or an epitope thereof, such that binding of the antigen-specific receptor to antigen, or the epitope thereof, elicits an immune response. In some embodiments, the antigen-specific receptor has antigenic specificity for a cancer antigen, such as a tumor-specific antigen (TSA) or a tumor-associated antigen (TAA).

In some embodiments, the antigen-specific receptor is a chimeric antigen receptor (CAR). Generally, a CAR includes an antigen binding domain, e.g., a single-chain variable fragment (scFv) of an antibody, fused to a transmembrane domain and an intracellular domain. In some embodiments, the antigenic specificity of a CAR can be encoded by a scFv which specifically binds to the antigen, or an epitope thereof. CARs, and methods of making them, are known in the art.

In yet other embodiments, the immune receptor is expressed from an antibody construct cloned from a B cell, e.g., memory B cell or plasma cell and engineered to lack the 6^{th} exon of the antibody constant region/antibody secretion exon. In one exemplary embodiment, the pseudo-memory B cell can be converted by building a construct without the 6^{th} exon of the antibody constant region/antibody secretion exon.

In some embodiments, the antigen-binding molecule is expressed from a construct that comprises the coding sequence for an IgG1 constant region, wherein the coding sequence for the IgG1 constant region comprises the 5^{th} exon of the constant region but not the 6^{th} exon of the constant region.

In some embodiments, the antigen-binding molecule is expressed from a construct that comprises the coding sequence for an IgG1 constant region, wherein the coding sequence for the IgG1 constant region comprises the 6^{th} exon of the constant region. In some embodiments, the coding sequence for the IgG1 constant region comprising the 6 exon of the constant region does not comprise the 5^{th} exon of the constant region.

In other embodiments, the antigen-binding molecule comprises an immunoglobulin. The immunoglobulin encompassed by the present disclosure can be any of the five primary classes of immunoglobulins IgA, IgD, IgE, IgG, and IgM.

In some embodiments, the antigen-binding molecule comprises an antibody or a functional fragment thereof.

An antibody or a functional fragment thereof can be a polypeptide molecule that recognizes and binds to a complementary target antigen. An antibody or a functional fragment thereof of the present disclosure encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

Antibodies or functional fragments thereof can be naturally occurring or synthetic. A naturally-occurring antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

Antibodies can also be produced synthetically. For example, RNAs (e.g., stgRNA) or proteins, which are monoclonal antibodies, can be synthesized using synthetic genes by recovering the antibody genes from source cells, amplifying into an appropriate vector, and introducing the vector into a host to cause the host to express the RNA (e.g., stgRNA) or protein. In general, RNAs (e.g., stgRNA) or proteins can be cloned from any species of antibody-producing animal using suitable oligonucleotide primers and/or hybridization probes. Recombinant techniques can be used to generate antibodies and antibody fragments, including non-endogenous species. In some embodiments, synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

In some embodiments, an antigen-binding molecule can be a therapeutic antibody or antigen-binding fragment thereof. A therapeutic antibody or antigen-binding fragment thereof can be a drug candidate or an FDA approved drug or therapeutic, such as a monoclonal antibody that is approved by the FDA for therapeutic use. Non-limiting examples of FDA approved monoclonal antibodies are provided in Table 1.

TABLE 1
Exemplary FDA Approved Therapeutic Monoclonal
Antibodies and other immunotherapies.

| Antibody | Brand name | Type | Target |
|---|---|---|---|
| abciximab | ReoPro | chimeric Fab | GPIIb/IIIa |
| adalimumab | Humira | fully human | TNF |
| adalimumab-atto | Amjevita | fully | TNF |
| | | human, biosimilar | |
| ado-trastuzumab | Kadcyla | humanized, | HER2 |
| emtansine | | antibody-drug | |
| | | conjugate | |
| alemtuzumab | Campath, | humanized | CD52 |
| | Lemtrada | | |
| alirocumab | Praluent | fully human | PCSK9 |
| atezolizumab | Tecentriq | humanized | PD-L1 |
| atezolizumab | Tecentriq | humanized | PD-L1 |
| avelumab | Bavencio | fully human | PD-L1 |
| basiliximab | Simulect | chimeric | IL2RA |
| belimumab | Benlysta | fully human | BLyS |
| bevacizumab | Avastin | humanized | VEGF |
| bezlotoxumab | Zinplava | fully human | Clostridium |
| | | | difficile |
| | | | toxin B |
| blinatumomab | Blincyto | mouse, bispecific | CD19 |
| brentuximab | Adcetris | chimeric, | CD30 |
| vedotin | | antibody-drug | |
| | | conjugate | |
| brodalumab | Siliq | chimeric | IL17RA |
| canakinumab | Ilaris | fully human | IL1B |
| capromab pendetide | ProstaScint | murine, | PSMA |
| | | radiolabeled | |
| certolizumab pegol | Cimzia | humanized | TNF |
| cetuximab | Erbitux | chimeric | EGFR |
| daclizumab | Zenapax | humanized | IL2RA |
| daclizumab | Zinbryta | humanized | IL2R |
| daratumumab | Darzalex | fully human | CD38 |
| denosumab | Prolia, | fully human | RANKL |
| | Xgeva | | |
| dinutuximab | Unituxin | chimeric | GD2 |
| dupilumab | Dupixent | fully human | IL4RA |
| durvalumab | Imfinzi | fully human | PD-L1 |
| eculizumab | Soliris | humanized | Complement |
| | | | component 5 |
| elotuzumab | Empliciti | humanized | SLAMF7 |
| evolocumab | Repatha | fully human | PCSK9 |
| golimumab | Simponi | fully human | TNF |
| golimumab | Simponi | fully human | TNF |
| | Aria | | |
| ibritumomab | Zevalin | murine, | CD20 |
| tiuxetan | | radioimmunotherapy | |
| idarucizumab | Praxbind | humanized Fab | dabigatran |
| infliximab | Remicade | chimeric | TNF alpha |
| infliximab-abda | Renflexis | chimeric, biosimilar | TNF |
| infliximab-dyyb | Inflectra | chimeric, biosimilar | TNF |
| ipilimumab | Yervoy | fully human | CTLA-4 |
| ixekizumab | Taltz | humanized | IL17A |
| mepolizumab | Nucala | humanized | IL5 |
| natalizumab | Tysabri | humanized | alpha-4 |
| | | | integrin |
| necitumumab | Portrazza | fully human | EGFR |
| nivolumab | Opdivo | fully human | PD-1 |
| nivolumab | Opdivo | fully human | PD-1 |
| obiltoxaximab | Anthem | chimeric | Protective |
| | | | antigen of |
| | | | the Anthrax |
| | | | toxin |
| obinutuzumab | Gazyva | humanized | CD20 |
| ocrelizumab | Ocrevus | humanized | CD20 |
| ofatumumab | Arzerra | fully human | CD20 |
| olaratumab | Lartruvo | fully human | PDGFRA |
| omalizumab | Xolair | humanized | IgE |
| palivizumab | Synagis | humanized | F protein |
| | | | of RSV |
| panitumumab | Vectibix | fully human | EGFR |
| pembrolizumab | Keytruda | humanized | PD-1 |
| pertuzumab | Perjeta | humanized | HER2 |
| ramucirumab | Cyramza | fully human | VEGFR2 |
| ranibizumab | Lucentis | humanized | VEGFR1, |
| | | | VEGFR2 |
| raxibacumab | Raxibacumab | fully human | Protective |
| | | | antigen |
| | | | of Bacillus |
| | | | anthracis |
| reslizumab | Cinqair | humanized | IL5 |
| rituximab | Rituxan | chimeric | CD20 |
| secukinumab | Cosentyx | fully human | IL17A |
| siltuximab | Sylvant | chimeric | IL6 |
| tocilizumab | Actemra | humanized | IL6R |
| tocilizumab | Actemra | humanized | IL6R |
| trastuzumab | Herceptin | humanized | HER2 |
| ustekinumab | Stelara | fully human | IL12 |
| ustekinumab | Stelara | fully human | IL12, IL23 |
| vedolizumab | Entyvio | humanized | integrin |
| | | | receptor |
| sarilumab | Kevzara | fully human | IL6R |
| rituximab and | Rituxan | chimeric, co- | CD20 |
| hyaluronidase | Hycela | formulated | |
| guselkumab | Tremfya | fully human | IL23 |
| inotuzumab | Besponsa | humanized, | CD22 |
| ozogamicin | | antibody-drug | |
| | | conjugate | |
| adalimumab-adbm | Cyltezo | fully | TNF |
| | | human, biosimilar | |
| gemtuzumab | Mylotarg | humanized, | CD33 |
| ozogamicin | | antibody-drug | |
| | | conjugate | |
| bevacizumab-awwb | Mvasi | humanized, | VEGF |
| | | biosimilar | |
| benralizumab | Fasenra | humanized | interleukin-5 |
| | | | receptor |
| | | | alpha subunit |
| emicizumab-kxwh | Hemlibra | humanized, | Factor |
| | | bispecific | IXa, Factor |
| | | | X |
| trastuzumab-dkst | Ogivri | humanized, | HER2 |
| | | biosimilar | |
| infliximab-qbtx | Ixifi | chimeric, biosimilar | TNF |
| ibalizumab-uiyk | Trogarzo | humanized | CD4 |
| tildrakizumab-asmn | Ilumya | humanized | IL23 |
| burosumab-twza | Crysvita | fully human | FGF23 |
| erenumab-aooe | Aimovig | fully human | CGRP |
| | | | receptor |

In some embodiments, an antigen-binding molecule can be similar to an FDA approved therapeutic monoclonal antibody. In some such cases, an antigen-binding molecule can have at least 75% identity, at least 80% identity, at least 85% identity, at least 90%/identity, or at least 95% identity to an FDA approved therapeutic monoclonal antibody or a range between any two foregoing values. In some embodiments, an antigen-binding molecule can have a heavy chain variable region that is at least 75% identity, at least 80% identity, at least 85% identity, at least 90% identity, or at least 95% identity to the heavy chain variable region of an FDA approved therapeutic monoclonal antibody or a range between any two foregoing values. In some embodiments, an antigen-binding molecule can have a heavy chain constant region that is at least 75% identity, at least 80% identity, at least 85% identity, at least 90% identity, or at least 95% identity to the heavy chain constant region of an FDA approved therapeutic monoclonal antibody or a range between any two foregoing values. In some embodiments, an antigen-binding molecule can have a light chain variable region that is at least 75% identity, at least 80% identity, at least 85% identity, at least 90% identity, or at least 95% identity to a light chain variable region of an FDA approved therapeutic monoclonal antibody or a range between any two foregoing values. In some embodiments, an antigen-binding molecule can have a light chain constant region that is at least 75% identity, at least 80% identity, at least 85% identity, at least 90% identity, or at least 95% identity to the light chain constant region of an FDA approved therapeutic monoclonal antibody or a range between any two foregoing values. In some embodiments, an antigen-binding molecule can be an antibody or antigen-binding fragment thereof that has a target that is the same as the target of an FDA approved therapeutic monoclonal antibody.

In some embodiments, the antibody is a monoclonal antibody, a polyclonal antibody, a multi-specific antibody, a bi-specific antibody, a chimeric antigen receptor, an oligoclonal antibody, a synthetic antibody, a recombinant antibody, a chimeric antibody, a heterochimeric antibody, or a humanized antibody. In some specific embodiments, the antibody is an oligoclonal antibody. An oligoclonal antibody generally refers to a collection of antibodies that are derived from a few clones. In some embodiments, the antibody or a functional fragment thereof comprises a monoclonal antibody or a polyclonal antibody. In other embodiments, functional fragments of any of the antibodies herein are also contemplated. Further disclosure regarding antibodies, and antigen-binding fragments thereof, that may be encoded as the antigen-binding molecule of interest in the nucleic acid construct is provided elsewhere in the disclosure herein infra.

Also contemplated herein is the use of an AVIMER® as an antigen or antigen binding moiety. The term “AVIMER®” can refer to a class of therapeutic proteins of human origin, which can be unrelated to antibodies and antibody fragments, and can be composed of several modular and reusable binding domains, referred to as A-domains (also referred to as class A module, complement type repeat, or LDL-receptor class A domain). They can be developed from human extracellular receptor domains by in vitro exon shuffling and phage display (Silverman et al., 2005, Nat. Biotechnol. 23:1493-1494; Silverman et al., 2006, Nat. Biotechnol. 24:220). The resulting proteins can contain multiple independent binding domains that can exhibit improved affinity and/or specificity compared with single-epitope binding proteins. Each of the known 217 human A-domains can include ˜35 amino acids (˜4 kDa); and these domains can be separated by linkers that can average five amino acids in length. Native A-domains fold quickly and efficiently to a uniform, stable structure mediated primarily by calcium binding and disulfide formation. A conserved scaffold motif of only 12 amino acids can be required for this common structure. The end result can be a single protein chain containing multiple domains, each of which represents a separate function. Each domain of the proteins can bind independently, and the energetic contributions of each domain can be additive.

Antigen-binding polypeptides can also include heavy chain dimers such as, for example, antibodies from camelids and sharks. Camelid and shark antibodies can include a homodimeric pair of two chains of V-like and C-like domains (neither has a light chain). Since the V_{H} region of a heavy chain dimer IgG in a camelid does may not have to make hydrophobic interactions with a light chain, the region in the heavy chain that normally contacts a light chain can be changed to hydrophilic amino acid residues in a camelid. V_{H} domains of heavy-chain dimer IgGs can be called V_{HH} domains. Shark Ig-NARs can include a homodimer of one variable domain (termed a V-NAR domain) and five C-like constant domains (C-NAR domains). In camelids, the diversity of antibody repertoire can be determined by the CDRs 1, 2, and 3 in the V_{H} or V_{HH} regions. The CDR3 in the camel V_{HH} region can be characterized by its relatively long length, averaging 16 amino acids (Muyldermans et al., 1994, Protein Engineering 7(9): 1129). Ibis can be in contrast to CDR3 regions of antibodies of many other species. For example, the CDR3 of mouse V_{H} can have an average of 9 amino acids. Libraries of camelid-derived antibody variable regions, which can maintain the in vivo diversity of the variable regions of a camelid, can be made by, for example, the methods disclosed in U.S. Patent Application Ser. No. 20050037421.

In certain embodiments, the antigen-binding molecule comprises a first segment encoding a first variable region and a second segment encoding a second variable region. Thus, in some embodiments, the nucleic acid construct of the present disclosure encoding the antigen-binding molecule (e.g., the third nucleic acid construct) is configured as a single chain comprising a first segment encoding a first variable region and a second segment encoding a second variable region. In some embodiments, the third nucleic acid construct comprises a linker that operably links the first segment and the second segment. The linker can be an autoproteolytic peptide such as, without being limited to, P2A, E2A, F2A, and T2A. Alternatively, the linker can be, without being limited to, an internal ribosome entry site (IRES) or a thrombin cleavage domain.

In some embodiments, the nucleic acid construct of the present disclosure comprises a nucleic acid sequence encoding a reporter protein. In some embodiments, the nucleic acid sequence encoding a reporter protein is referred to as the fourth nucleic acid sequence. In some embodiments, the nucleic acid sequence encoding a reporter protein is operably linked to a promoter, which is also referred to as the fourth promoter in some instances. In some embodiments, the nucleic acid sequence encoding a reporter protein is operably linked to a constitutive promoter. In some embodiments, the nucleic acid sequence encoding a reporter protein is operably linked to a constitutive promoter, such as but not limited to, a CMV promoter.

A reporter protein as used herein generally refers to a protein that can be used to measure gene expression and generally produces a measurable signal such as fluorescence, color, luminescence, or resistance to a selective reagent. In some embodiments, the reporter protein comprises a fluorescent protein. Examples of fluorescent proteins that may be used include, e.g., green fluorescent protein (GFP), eGFP, red fluorescent protein (RFP), blue fluorescent protein (BFP), yellow fluorescent protein (YFP), cyan fluorescent protein (CFP), luciferase, Sirius, Azurite, eBFP2, mTurquoise, eCFP, Cerulean, mTFP1, mUkG1, mAG1, AcGFP, mWasabi, EmGFP, eYFP, Topaz, SYFP2, Venus, Citrine, mKO, mKO2, mOrange, mOrange2, LSSmOrange, PSmOrange, and PSmOrange2, mStrawberry, mRuby, mCherry, mRaspberry, tdTomato, mKate, mKate2, mPlum, mNeptune, T-Sapphire, mAmetrine, mKeima, E2-Orange, E2-Red/Green, E2-Crimson, and ZsGreen.

In other embodiment, a reporter protein can be a selection marker, for example, proteins that confer resistance to selective reagents such as, but not limited to neomycin, puromycin, hygromycin, DHFR, GPT, zeocin, and histidinol are useful non-limiting exemplary selection markers. In yet other embodiments, the reporter protein is suitable for use in a colorimetric assay.

In one exemplary embodiment, the reporter protein is a fluorescent protein. In other exemplary embodiments, the reporter protein comprises a GFP, eGFP, RFP, BFP, YFP, CFP, citrine, and luciferase. In additional exemplary embodiments, the reporter protein comprises β-glucuronidase (GUS), lacZ/beta-galactosidase, Chloramphenicol acetyltransferase (CAT), puromycin N-acetyltransferase (PAC), dihydrofolate reductase (DHFR), Sh ble protein (resistant to zeocin), and hygromycin B phosphotransferase. However, it is understood that other reporter proteins are generally known and used in the art and are also encompassed by the present disclosure.

Some exemplary nucleic acid constructs of the present disclosure are illustrated herein.

In some embodiments, the nucleic acid constructs of the present disclosure comprise one or more of the following: a first nucleic acid sequence encoding a self-targeting guide RNA (stgRNA), a second nucleic acid sequence encoding a CRISPR-associated (Cas) protein, and/or a third nucleic acid sequence encoding an antigen-binding molecule of interest. In some embodiments, each of the first, the second, and the third nucleic acid sequence is operably linked to a promoter, as described below. In some embodiments, the promoter operably linked to the first, the second, and the third nucleic acid sequence is each denoted as the first, the second, and the third promoter, respectively.

In some embodiments, the promoter linked to the nucleic acid sequence encoding the Cas protein (i.e., the second promoter) can be induced by an immune response of the engineered cell as described below. In some embodiments, the promoter linked to the nucleic acid sequence encoding the Cas protein (i.e., the second promoter) can be induced by an immune response of the engineered cell as described below. In certain embodiments, the coding sequence for the Cas protein is operably linked to a NFkB inducible promoter. In some embodiments, the nucleic acid constructs provided herein further comprises a fourth nucleic acid sequence encoding a reporter protein.

In some embodiments, at least one of the first, the second, the third, and the fourth nucleic acid sequences is heterologous or non-heterologous. The term heterologous as used herein generally means that the nucleic acid sequences provided herein are derived from a different source from each other or from a recipient. In some instances, the recipient can be the recombinant or engineered cells discussed herein. In some embodiments, all of the first, the second, the third, and the fourth nucleic acid sequences is heterologous or non-heterologous.

In some embodiments, the first, the second, the third, and the fourth nucleic acid sequences are in the same construct. In other embodiments, the first, the second, the third, and the fourth nucleic acid sequences are in separate constructs.

In some exemplary embodiments, the nucleic acid constructs provided herein comprises the first and the second nucleic acid sequences in the same construct, and the first and the second nucleic acid sequences are heterologous. In other exemplary embodiments, the nucleic acid constructs provided herein comprises the first, the second, and the third nucleic acid sequences in the same construct, and the first, the second, and the third nucleic acid sequences are heterologous. In additional exemplary embodiments, the nucleic acid constructs provided herein comprises the first, the second, and the fourth nucleic acid sequences in the same construct, and the first, the second, and the fourth nucleic acid sequences are heterologous. In further exemplary embodiments, the nucleic acid constructs provided herein comprises the first, the second, the third, and the fourth nucleic acid sequences in the same construct, and the first, the second, the third, and the fourth nucleic acid sequences are heterologous.

In alternative embodiments, the present disclosure provides nucleic acid constructs comprising a nucleic acid sequence encoding an antigen-binding molecule of interest, and a nucleic acid sequence encoding an enzyme capable of mediating somatic hypermutation (SHM). In certain embodiments, the coding sequence for the SHM enzyme is operably linked to an inducible promoter as described herein. In one specific embodiment, the inducible promoter is an NFkB inducible promoter. In some embodiments, the nucleic acid sequence encoding an antigen-binding molecule of interest and nucleic acid sequence encoding an enzyme capable of mediating SHM are in the same construct. In other embodiments, the nucleic acid sequence encoding an antigen-binding molecule of interest and nucleic acid sequence encoding an enzyme capable of mediating SHM are in the separate constructs. In some specific embodiments, the SHM enzyme coding sequence is put under inducible control to enable selective activation of somatic hypermutation in an isolated clone of engineered cells that express the antigen-binding molecule of interest.

In other alternative embodiments, the nucleic acid constructs of the present disclosure comprise: a first nucleic acid sequence encoding a self-targeting guide RNA (stgRNA), a second nucleic acid sequence encoding a CRISPR-associated (Cas) protein, and a nucleic acid sequence encoding an enzyme capable of mediating SHM. In certain embodiments, the nucleic acid constructs also comprises a nucleic acid sequence encoding an antigen-binding molecule of interest. In certain embodiments, the stgRNA, the Cas protein, and the SHM enzyme are each independently operably linked to a inducible promoter. In some embodiments, the Cas protein and the SHM enzyme are operably linked together by a self-cleaving linker provided herein. In some embodiments, the linker can be an internal ribosome entry site (IRES). In other embodiments, the linker can be a thrombin cleavage domain.

In some embodiments, the nucleic acid constructs are provided as a set of nucleic acid constructs, each comprising one or more of the nucleic acid sequences described herein.

### B. Target Antigen

One aspect of the present disclosure relates to contacting an engineered cell expressing the nucleic acid constructs or an antigen-binding molecule of interest (e.g., a BCR, an antibody, and antigen-binding fragment of an antibody, etc.) with a target antigen. An antigen encompassed by the present disclosure can include, but is not limited to, a protein, a peptide, an antibody (or a fragment thereof), a small molecule, or a pathogen. In some specific embodiments, the antigen comprises a protein, a viral-like particle, and a nanoparticle. In other specific embodiments, the antigen comprises pathogen-associated molecular patterns (PAMP), complement proteins (e.g., complement proteins on the microbes), epitope-antibody complexes, epitopes bound to MHC, and cytokines. In an exemplary embodiment, the antigen is a protein. Examples of target antigens of an antigen-binding molecule of interest, e.g., an antibody, or antigen-binding fragment thereof, are provided throughout the disclosure herein.

In some embodiments, a target antigen is coupled to a reporter oligonucleotide, which is described in detail below. In some embodiments, the use of antigens coupled to a reporter oligonucleotide allows the detection of the specific antigen or antigens in a panel of antigens are recognized by a given clone of engineered cells (e.g., engineered immune cells, engineered B cells). In other embodiments, the use of antigens coupled to a reporter oligonucleotide allows the detection of the specific antigen or antigens in a panel of antigens are recognized by specific progeny of a clone by inferring the presence of the antigen coupled to a reporter oligonucleotide and de novo mutations induced by an SHM enzyme described herein.

In some embodiments, an antigen can be a biomolecule, such as a biologic therapeutic molecule. Examples of biologic therapeutic molecules can be, for example, a drug-reactive antibody or anti-drug antibody that is produced from a living organism or that contains one or more components of a living organism. A biologic therapeutic molecule can be derived from a human, animal, or microorganism using biotechnology techniques. Examples of biologic therapeutic molecules can include, for example, an immunological molecule (e.g. an antibody (such as a monoclonal antibodies), a fusion protein, a protein product of a gene therapy, a peptide, or other biologic molecule. In other embodiments, the antigen is a cell, for instance, a cell expressing a surface antigen.

In some embodiments, an antigen can be an antibody or antigen-binding fragment thereof. In some embodiments, an antigen can be an antibody-drug conjugate. In some embodiments, an antigen can be a therapeutic antibody or antigen-binding fragment thereof (e.g., a monoclonal antibody). In some embodiments, an antigen can be the target antigen of a therapeutic antibody or antigen-binding fragment thereof (e.g., a monoclonal antibody). Some non-limiting exemplary FDA approved therapeutic antibodies are provided in Table 1.

### C. Promoters

Any one of the nucleic acid sequences provided herein can each be operably linked to a promoter. In some embodiments, the promoter comprises a strong and/or constitutive promoter. The strength of a promoter generally refers to the rate of transcription of a gene controlled by this promoter. In some embodiments, a strong promoter means the rate of transcription of a gene controlled by this promoter is high. The strength of a promoter is relative and can generally be determined empirically. A strong promoter can be constitutive or inducible (i.e., regulated). A constitutive promoter as used herein generally refers to an unregulated promoter that allows for continual transcription of its associated gene. For instance, a constitutive promoter's activity is dependent on the availability of RNA polymerase holoenzyme but is not affected by any transcription factors. A constitutive can have any level of strengths. In some embodiments, the constitutive promoter encompassed herein is a strong promoter.

Some non-limiting, exemplary strong and/or constitutive promoters encompassed by the present disclosure include a CMV promoter, an EF1a promoter, a SV39 promoter, a SV40 promoter, a Ubc promoter, a human beta actin promoter, a CAG promoter, a PGK1 promoter, a TRE promoter, a UAS promoter, an Ac5 promoter, a polyhedrin promoter, a CaMKIIa promoter, a GAL 1 promoter, a GAL 10 promoter, a TEF1 promoter, a GDS promoter, a ADH1 promoter, a CaMV35S promoter, a Ubi promoter, a H1 promoter, and a U6 promoter.

In contrast, inducible promoters are only active under specific circumstances. Inducible promoters are generally known and used in the art. Inducible promoters can also be used in any of the nucleic acid constructs provided herein. For instances, in some embodiments, inducible promoters can be regulated by positive or negative control. In certain non-limiting exemplary embodiments, inducible promoters can be regulated by chemical agents, temperature, and light. In other non-limiting exemplary embodiments, inducible promoters can be regulated by transcription factors. In some embodiments, the transcription factors are activated by an immune response of a cell. Non-limiting, exemplary inducible promoters include a nucleotide factor kappa-B (NFkB) promoter, a MyD88 promoter, an interleukin (IL)-2 promoter, and a JAK/STAT promoter, a Bcl-xL promoter, a c-Rel promoter, a Myc promoter, a NFAT promoter, a mTORC1 promoter, a CD40 promoter, a TLR9 promoter, a Btk promoter, a PI3K promoter, an actin-related protein 2/3 (Arp2/3) promoter, and an IL-4 promoter. In some exemplary embodiments, the Arp2/3 promoter is used where antigen-presenting cells are used to activate the B cells with constructs provided herein. In other exemplary embodiments, an IL-4 promoter is used where cells are stimulated with IL-4 to induce cytidine deaminase (AID) activity. In some embodiments, the construct further comprises an enhancer element that is regulated by a transcription factor disclosed herein.

### D. Linkers

In some embodiments, one or more domains of the nucleic acid sequences provided herein are linked via a linker. For instances, in some embodiments, a single-chain antigen-binding molecule provided herein comprises at least one linker. In other embodiments, the coding sequence for an SHM enzyme is operably linked to a Cas protein coding sequence via a linker.

A linker as used herein refers to a nucleic acid sequence and/or a peptide encoded by the nucleic acid sequence, which is capable of covalently joining the functional domains (e.g., various segments in the nucleic acid sequences comprised in the constructs provided herein) or to release them under desired conditions (e.g., cleavable linkers). In certain embodiments, linkers may offer many other advantages for the production of fusion proteins, such as improving biological activity, increasing expression yield, and achieving desirable pharmacokinetic profiles. In some embodiments, a linker encompassed by the present disclosure comprises naturally occurring sequences, empirically designed artificial sequences, or a combination of both. Empirically designed linkers are generally classified into 3 categories according to their structures: flexible linkers, rigid linkers, and in vivo cleavable linkers. Numerous linkers are known in the art. For instance, various linkers are reviewed by Chen et al. (Fusion Protein Linkers: Property, Design and Functionality, Adv Drug Deliv Rev. 2013 Oct. 15; 65(10): 1357-1369). A skilled person would know how to choose or empirically design a suitable linker for a specific application with the guidance available in the field.

In some embodiments, the linker is a cleavable linker. In some embodiments, the linker comprises an autoproteolytic peptide. Non-limiting exemplary autoproteolytic peptides comprise P2A, E2A, F2A, T2A, and internal ribosome entry site (IRES).

### E. Vectors

In some embodiments, the nucleic acid constructs of the present disclosure is incorporated into an expression vector. It is understood by one skilled in the art that the term “vector” generally refers to a recombinant polynucleotide construct designed for transfer between host cells, and that can be used for the purpose of transformation, e.g., the introduction of heterologous DNA into a host cell. As such, in some embodiments, the vector can be a replicon, such as a plasmid, phage, or cosmid, into which another DNA segment can be inserted so as to bring about the replication of the inserted segment. In some embodiments, the expression vector can be an integrating vector.

In some embodiments, the expression vector can be a viral vector. As will be appreciated by one of skill in the art, a viral vector can be either a nucleic acid molecule (e.g., a transfer plasmid) that includes virus-derived nucleic acid elements that generally can facilitate transfer of the nucleic acid molecule or integration into the genome of a cell or can be a viral particle that mediates nucleic acid transfer. Viral particles will generally include various viral components and sometimes also host cell components in addition to nucleic acid(s). In some embodiments, a viral vector can be either a virus or viral particle capable of transferring a nucleic acid into a cell or to the transferred nucleic acid itself. Viral vectors and transfer plasmids contain structural and/or functional genetic elements that are primarily derived from a virus. As such, a retroviral vector can be a viral vector or plasmid containing structural and functional genetic elements, or portions thereof, that are primarily derived from a retrovirus. Similarly, a lentiviral vector can be a viral vector or plasmid containing structural and functional genetic elements, or portions thereof, including LTRs that are primarily derived from a lentivirus, which is a genus of retrovirus.

The nucleic acid sequences encoding the RNAs (e.g., stgRNA) or proteins as disclosed herein can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the RNAs (e.g., stgRNA) or proteins disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to expression cassettes including a nucleic acid constructs and/or a nucleic acid sequence encoding the RNAs (e.g., stgRNA) or proteins disclosed herein. The expression cassette generally contains coding sequences and sufficient regulatory information to direct proper transcription and/or translation of the coding sequences in a recipient cell, in vivo and/or ex vivo. The expression cassette can be inserted into a vector for targeting to a desired host cell and/or into an individual. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid construct where one or more nucleic acid sequences has been linked in a functionally operative manner, i.e., operably linked.

Also provided herein are plasmids or viruses containing one or more of the nucleic acid constructs and/or the nucleic acid sequences encoding any RNA (e.g., stgRNA) or protein as disclosed herein. The nucleic acid constructs can be contained within a vector that is capable of directing their amplification or expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferrd, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

In some embodiments, the vectors of the present disclosure further include an inducible cellular switch. In some embodiments, the inducible cellular switch allows control of the expression of the nucleic acid constructs of the disclosure by contacting a cell comprising the vector with a molecule or reagent that can turn on or off the switch. In other embodiments, these inducible cellular switches are included to improve the safety and controllability of cell therapy. The inducible cellular switches can be in the form of, without being limited to, synthetic receptors, protein-based switches, genetic circuits, and genome editing tools. In certain embodiments, the inducible cellular switches comprise drug-inducible switches. In some embodiments, the inducible cellular switches comprise genetic circuits that endow additional spatiotemporal control. In other embodiments, the inducible cellular switches can be triggered by signaling pathways. Inducible cellular switches are generally known and used in the art, and thus, a skilled person would know how to choose or design a suitable inducible cellular switches for specific uses. Non-limiting exemplary inducible cellular switches comprise rimiducid response element, tetracycline response element, rapamycin response element, ecdysone response element, mifepristone response element, light-based response system, CD24, and HSV-TK/CDK1 pairing. Such vectors can be used to develop or manufacture clinically viable therapeutic compositions (e.g., for cellular therapy).

F. Recombinant and/or Engineered Cells

The nucleic acid constructs of the present disclosure can be introduced into a host cell, such as, for example, a human T lymphocyte, to produce a recombinant cell or engineered cell containing the nucleic acid constructs. Introduction of the nucleic acid constructs of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid constructs can be delivered by viral or non-viral delivery vehicles known in the art. For example, the nucleic acid constructs can be stably integrated in the host genome, or can be episomally replicating, or present in the engineered ell as a mini-circle expression vector for transient expression. Accordingly, in some embodiments, the nucleic acid constructs is maintained and replicated in the engineered cell as an episomal unit. In some embodiments, the nucleic acid constructs is stably integrated into the genome of the engineered cell. Stable integration can be achieved using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the nucleic acid constructs is present in the engineered ell as a mini-circle expression vector for transient expression.

The nucleic acid constructs can be encapsulated in a viral capsid or a lipid nanoparticle, or can be delivered by viral or non-viral delivery means and methods known in the art, such as electroporation. For example, introduction of nucleic acids into cells can be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present disclosure that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell or engineered cell is a prokaryotic cell or a eukaryotic cell. In some embodiments, the cell is in vivo. In some embodiments, the cell is ex vivo. In some embodiments, the cell is in vitro. In some embodiments, the engineered cell is a eukaryotic cell. In some embodiments, the engineered cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the cell is a stem cell. In some embodiments, the cell is a hematopoietic stem cell. Examples of cells are provided thoughout the disclosure herein.

In some embodiments, the recombinant cell or engineered cell is an immune cell, e.g., a lymphocyte (e.g., a T cell or NK cell), or a dendritic cell. In some embodiments, the immune cell is a B cell, a monocyte, a natural killer (NK) cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), or other T cell. In some embodiments, the immune cell is a T lymphocyte. In some embodiments, the cell is a precursor T cell or a T regulatory (Treg) cell. In some embodiments, the cell is a CD34+, CD8+, or a CD4+ cell. In some embodiments, the cell is a CD8+T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from an individual. In some embodiments, the subject is a human patient.

In some specific embodiments, the engineered cell is a B cell. In certain embodiments, a cell (e.g., B cell) is engineered to express one or more of the stgRNA, the Cas protein, the antigen-binding molecule, and the reporter protein provided herein. In one exemplary embodiment, a cell (e.g., B cell) is engineered to express all of the stgRNA, the Cas protein, the antigen-binding molecule, and the reporter protein provided herein. In another exemplary embodiment, a cell (e.g., B cell) is engineered to express all of the stgRNA, the Cas protein, the antigen-binding molecule, and the reporter protein provided herein, and the coding sequence for the Cas protein is operably linked to a NFkB inducible promoter. In yet another exemplary embodiment, a cell (e.g., B cell) is engineered to further express an SHM enzyme. In certain embodiments, the expression of the SHM enzyme is operably linked to the Cas protein driven by a NFkB inducible promoter. In other embodiments, a cell (e.g., B cell) is engineered to express an antigen-binding molecule of interest and an SHM enzyme. In certain embodiments, the SHM enzyme expressed by the engineered cell enables antigen specific somatic hypermutation within the antigen-binding molecule of interest and rapid selection of additional mutations that result in enhanced affinity for the antigen.

In other exemplary embodiments, a cell (e.g., B cell) is engineered to express one or more of the stgRNA, the Cas protein, and the reporter protein described herein, and the cell comprises a nucleic acid sequence encoding the antigen-binding molecule of interest. In certain embodiments, the nucleic acid sequence encoding an antigen-binding molecule of interest is non-heterologous to the cell.

In some specific embodiments, a cell (e.g., B cell) is engineered to express a single antigen-binding molecule (e.g., a single B cell receptor (BCR)). In other embodiments, a cell (e.g., B cell) is engineered to express more than one antigen-binding molecule. For instance, in certain exemplary embodiments, an antigen-binding molecule of interest may comprise a single light chain and two heavy chains, and many light chains can pair with multiple unique heavy chains. Thus, in such embodiments, a single engineered cell can express more than one antigen-binding molecules, for example, more than one BCRs.

In another aspect, some embodiments of the disclosure relate to methods for making an engineered cell, including (a) providing a cell capable of protein expression and (b) contacting the provided cell with a nucleic acid construct of the disclosure.

In another aspect, provided herein are cell cultures including at least one engineered cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one engineered cell as disclosed herein are also within the scope of this application. Methods and systems suitable for generating and maintaining cell cultures are known in the art.

### G. Compositions and Pharmaceutical Compositions

The nucleic acids constructs, engineered cells, vectors, expression cassettes, and/or cell cultures of the disclosure can be incorporated into compositions, including pharmaceutical compositions. Further, the antigen-binding molecule selected or made by the methods provided herein can also be incorporated into compositions, including pharmaceutical compositions. For example, in some embodiments, the present disclosure provides a composition comprising a plurality of the vectors described herein. In some embodiments, the present disclosure provides a composition comprising a plurality of the nucleic acid constructs provided herein. In other embodiments, the present disclosure provides a composition comprising a plurality of the engineered cells described herein. In yet other embodiments, the present disclosure provides a composition comprising an antigen-binding molecule selected or made by the methods provided herein. In certain embodiments, the nucleic acids constructs, engineered cells, vectors, expression cassettes, and/or cell cultures as described herein can be included in compositions suitable for various downstream applications.

In other embodiments, the compositions provided herein further comprise a pharmaceutically acceptable excipient. The pharmaceutical compositions provided herein can be in any form that allows for the composition to be administered to an individual. In some specific embodiments, the pharmaceutical compositions are suitable for human administration. As used herein, the term “pharmaceutically acceptable” means approved by a regulatory agency of the Federal or a state government or listed in the U.S. Pharmacopeia or other generally recognized pharmacopeiae for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual or an animal (some non-limiting examples include a human, or a mammal). In some embodiments, the individual is a human.

The terms “administration” and “administering”, as used herein, refer to the delivery of a bioactive composition or formulation by an administration route comprising, but not limited to, intranasal, transdermal, intravenous, intra-arterial, intramuscular, intraperitoneal, subcutaneous, intramuscular, oral, and topical administration, or combinations thereof. The term includes, but is not limited to, administering by a medical professional and self-administering.

In some embodiments, the pharmaceutical compositions of the present disclosure are formulated to be suitable for the intended route of administration to an individual. For example, the pharmaceutical composition can be formulated to be suitable for parenteral, intraperitoneal, colorectal, intraperitoneal, and intratumoral administration. In some embodiments, the pharmaceutical composition can be formulated for intravenous, oral, intraperitoneal, intratracheal, subcutaneous, intramuscular, topical, or intratumoral administration. One of ordinary skilled in the art will appreciate that the formulation should suit the mode of administration.

For example, pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In some embodiments, the composition should be sterile and should be fluid to the extent that easy syringability exists. It can be stabilized under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

### H. Kits

Also provided herein are kits for the practice of the methods described herein. A kit can include instructions for use thereof and one or more of the antigen-binding molecules, nucleic acid constructs, engineered cells, and other compositions as described and provided herein. For examples, some embodiments of the disclosure provide kits that include one or more of the antigen-binding molecules described herein, and instructions for use. In some embodiments, provided herein are kits that include one or more nucleic acid constructs, engineered cells, and other compositions as described herein and instructions for use thereof.

In some embodiments, the components of a kit can be in separate containers. In some other embodiments, the components of a kit can be combined in a single container.

In some embodiments, a kit can further include instructions for using the components of the kit to practice a method described herein. The instructions for practicing the method are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### Methods

A. Methods for Analyzing and/or Identifying an Antigen-Binding Molecule of Interest

The present disclosure further provides a method for analyzing, identifying, selecting, and/or affinity maturing an antigen-binding molecule of interest.

In some embodiments, the method comprises contacting at least one of the engineered cells expressing one or more of the stgRNA, the Cas protein, the antigen-binding molecule, and the reporter protein provided herein. In one exemplary embodiment, the method comprises contacting at least one of the engineered cells expressing all of the stgRNA, the Cas protein, the antigen-binding molecule, and the reporter protein provided herein. In another exemplary embodiment, the method comprises contacting at least one of the engineered cells expressing all of the stgRNA, the Cas protein, the antigen-binding molecule, and the reporter protein provided herein, and the coding sequence for the Cas protein is operably linked to a NFkB inducible promoter. In yet another exemplary embodiment, the method comprises contacting at least one of the engineered cells further expressing an SHM enzyme. In certain embodiments, the expression of the SHM enzyme is operably linked to the Cas protein driven by a NFkB inducible promoter. In other embodiments, the method comprises contacting at least one of the engineered cells expressing an antigen-binding molecule of interest and an SHM enzyme. In certain embodiments, the SHM enzyme expressed by the engineered cell enables antigen specific somatic hypermutation within the antigen-binding molecule of interest and rapid selection of additional mutations that result in enhanced affinity for the antigen.

In other exemplary embodiments, the method comprises contacting at least one of the engineered cells expressing one or more of the stgRNA, the Cas protein, and the reporter protein described herein, and the cell comprises a nucleic acid sequence encoding the antigen-binding molecule of interest. In certain embodiments, the nucleic acid sequence encoding an antigen-binding molecule of interest is non-heterologous to the cell.

In some specific embodiments, the method comprises contacting at least one of the engineered cells expressing a single antigen-binding molecule (e.g., a single B cell receptor (BCR)). In other embodiments, the method comprises contacting at least one of the engineered cells expressing more than one antigen-binding molecule.

In certain embodiments of any of the foregoing methods, the method comprises contacting at least one of the engineered cells provided herein with a target antigen of the antigen-binding molecule of interest.

In some embodiments, the binding of the target antigen with the antigen-binding molecule expressed by the engineered cells activates an immune response that induces expression of the Cas protein. In certain embodiments, the induced Cas protein introduces one or more mutations in the nucleic acid sequence of the stgRNA.

In some embodiments, the method further comprises determining the number of mutations introduced in the nucleic acid sequence of the stgRNA. In some embodiments, the method further comprises using the determined number of mutations to analyze the binding affinity of the antigen-binding molecule of interest for the antigen. In some embodiments, the determining comprises sequencing all or a part of the nucleic acid sequence encoding the stgRNA expressed by the at least one engineered cell.

In some embodiments, a method of affinity maturing an antigen-binding molecule of interest comprises contacting an engineered cell expressing the antigen-binding molecule of interest and an SHM enzyme with a target antigen of the antigen-binding molecule of interest. In some embodiments, the engineered cell expressing the antigen-binding molecule of interest and an SHM enzyme comprises a construct comprising (i) a nucleic acid sequence encoding the antigen-binding molecule of interest and (ii) a a nucleic acid sequence encoding an enzyme capable of mediating SHM, wherein the coding sequence for the SHM enzyme is operably linked to an inducible promoter, wherein the inducible promoter is induced by an immune stimulus or one or more stimuli. In one embodiment, the inducible promoter is induced by an immune response. Inducible promoters and SHM enzymes are described in further detail herein.

Without wishing to be bound by theory, binding of the target antigen to the antigen-binding molecule of interest can activate an immune response in the engineered cell, thereby inducing the inducible promoter to trigger expression of the SHM enzyme. The expressed SHM enzyme can induce somatic hypermutation into the antigen-binding molecule coding sequence. Thus, antigen-binding molecules that bind to the target antigen selectively undergo SHM and affinity maturation. Subsequently, the engineered cells may be partitioned and processed according to methods disclosed herein.

In certain embodiments, the identified antigen-binding molecule has higher affinity with a target antigen, e.g., as compared to an antigen-binding molecule expressed by a reference cell. In some embodiments, the identified antigen-binding molecule has higher affinity with a target antigen due to the mutations introduced in the antigen-binding molecule as a result of the SHM enzyme introduced in the engineered cell.

A plethora of different approaches, systems, and techniques for nucleic acid sequencing, including next-generation sequencing (NGS) methods, can be used to determine the nucleic acid sequences. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification.

Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLiD™ sequencing, Ion Torrent semiconductor sequencing, HeliScope single molecule sequencing, nanopore sequencing (see, e.g., Oxford Nanopore Technologies), and SMRT® sequencing.

Other examples of methods for sequencing genetic material include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing, MS-PET sequencing, whole transcriptome sequencing, and any combinations thereof.

Sequence analysis of the nucleic acid molecules can be direct or indirect. Thus, the sequence analysis substrate (which can be viewed as the molecule which is subjected to the sequence analysis step or process) can be a barcoded nucleic acid molecule or it can be a molecule which is derived therefrom (e.g., a complement thereof). Thus, for example, in the sequence analysis step of a sequencing reaction, the sequencing template can be the barcoded nucleic acid molecule or it can be a molecule derived therefrom. For example, a first and/or second strand DNA molecule can be directly subjected to sequence analysis (e.g., sequencing), i.e., can directly take part in the sequence analysis reaction or process (e.g., the sequencing reaction or sequencing process, or be the molecule which is sequenced or otherwise identified). Alternatively, a barcoded nucleic acid molecule can be subjected to a step of second strand synthesis or amplification before sequence analysis (e.g., sequencing or identification by another technique). The sequence analysis substrate (e.g., template) can thus be an amplicon or a second strand of a barcoded nucleic acid molecule.

In some embodiments, both strands of a double stranded molecule can be subjected to sequence analysis. In some embodiments, single stranded molecules can be sequenced.

In some embodiments, all or a part of the nucleic acid sequences encoding the stgRNA expressed by the at least one engineered cell can be determined by using a whole transcriptome sequencing technique, which generally involves sequencing the complete complement of transcripts in a sample, at a given time (often referred to as the transcriptome). Whole transcriptome sequencing generally uses high throughput sequencing technologies to sequence the entire transcriptome in order to get information about a sample's (e.g., an engineered cell provided herein) RNA content. Whole transcriptome sequencing can be done with a variety of platforms for example, the Genome Analyzer (Illumina, Inc., San Diego, Calif.) and the SOLiD™ Sequencing System (Life Technologies, Carlsbad, Calif.). However, any platform useful for whole transcriptome sequencing may be used. The term “RNA-Seq” or “transcriptome sequencing” refers to sequencing performed on RNA (or cDNA) instead of DNA, where generally, the primary goal is to measure expression levels, detect fusion transcripts, alternative splicing, and other genomic alterations that can be better assessed from RNA. RNA-Seq includes whole transcriptome sequencing as well as target specific sequencing.

In some embodiments, the method further comprises comparing the all or a part of the nucleic acid sequence encoding the stgRNA to the nucleic acid sequence encoding the stgRNA from a reference engineered cell.

In some embodiments, a reference engineered cell as used herein comprises an engineered cell that expresses an antigen-binding molecule that does not bind to the target antigen. In some embodiments, a reference engineered cell as used herein comprises an engineered cell that expresses an antigen-binding molecule that binds to other antigens. In other embodiments, a reference engineered cell as used herein comprises an engineered cell that does not express an antigen-binding molecule. In yet other embodiments, a reference engineered cell as used herein comprises an engineered cell whose the coding sequence for Cas protein is not switched on.

In certain embodiments wherein an engineered cell expresses the antigen-binding molecule of interest and an SHM enzyme, a reference engineered cell may express the antigen-binding molecule of interest but not the SHM enzyme.

In some embodiments, the at least one engineered cell comprises a population of engineered cells. Thus, in some embodiments, the method of selecting an antigen-binding molecule of interest further comprises partitioning the population of engineered cells into partitions of a plurality of partitions. In certain embodiments, a partition of the plurality of partitions comprises a single engineered cell.

In some embodiments, the plurality of partitions comprise a plurality of microwells or a plurality of droplets. In some embodiments, the partitioning occurs prior to contacting the at least one of the engineered cells a target antigen of the antigen-binding molecule of interest. In other embodiments, the partitioning occurs after contacting the at least one of the engineered cells with a target antigen of the antigen-binding molecule of interest. Systems and methods for partitioning are described in detail below.

In some embodiments, the partition of the plurality of partitions further comprises a plurality of nucleic acid barcode molecules. In certain embodiments, each of the nucleic acid barcode molecules comprises a partition-specific barcode sequence. Thus, in some embodiments, the method provided herein further comprises using a nucleic acid barcode molecule of the plurality of nucleic acid barcode molecules and an analyte of the engineered cell to generate a barcoded analyte of the engineered cell. In certain embodiments, at least a subset of the plurality of nucleic acid barcode molecules are attached to a bead. The bead may be a solid bead, a magnetic bead, or a gel bead. In certain embodiments, at least a subset of the plurality of nucleic acid barcode molecules are releasably attached to a gel bead. In some embodiments, the gel bead is degradable. Therefore, in some embodiments, single engineered cells derived from a population of engineered cells are individually partitioned to discrete droplets together with beads (e.g., gel beads) coupled with nucleic acid barcode molecules to generate droplets that contain a single engineered cell and a single bead. In other embodiments, single engineered cells are individually co-partitioned along with a bead (e.g., a gel bead) coupled to a nucleic acid barcode molecule, and other reagents into a partition (e.g., a droplet in an emulsion).

In some embodiments, at least a subset of the plurality of nucleic acid barcode molecules are releasably (e.g., reversibly) attached to a gel bead. In some embodiments, the method described herein further comprises releasing the nucleic acid barcode molecules from the gel bead prior to generating a first barcoded nucleic acid molecule, as described below. In some embodiments, the nucleic acid barcode molecules are released from the gel bead through degradation of the gel bead. In other embodiments, the nucleic acid barcode molecules are released from the gel bead through cleavage of a linkage between the nucleic acid barcode molecule and the gel bead.

In some embodiments, the nucleic acid barcode molecule further comprises a unique molecular identifier (UMI) sequence. In some embodiments, the UMI sequence of the nucleic acid barcode molecule differs from the UMI sequence of another nucleic acid barcode molecule of the plurality of nucleic acid barcode molecules in the partition. In some embodiments, the nucleic acid barcode molecule further comprises a functional sequence.

In some embodiments, the nucleic acid barcode molecule further comprises a capture sequence.

In some embodiments, the capture sequence comprises a sequence complementary to a capture handle sequence present on an analyte of interest, an extension product of an analyte of interest, or a reporter oligonucleotide coupled to the analyte of interest. As described in more detail below, in some embodiments, an analyte of interest, extension product thereof, or reporter oligonucleotide coupled thereto may further comprise a capture handle sequence that is complementary to the capture sequence of the nucleic acid barcode molecule. In some embodiments, the capture sequence comprises a template switch oligonucleotide (TSO) sequence. In other embodiments, the capture sequence comprises a polyT sequence. In yet other embodiments, the capture sequence comprises a reporter capture sequence complementary to a reporter capture handle present on a reporter oligonucleotide described herein.

In some embodiments, the target antigen is coupled to a reporter oligonucleotide, which is discussed in detail below. In some embodiments, the reporter oligonucleotide comprises a reporter barcode sequence. In certain embodiments, the reporter oligonucleotide further comprises a reporter capture handle and/or a functional sequence. In some embodiments, the reporter capture handle comprises a sequence that is complementary to the reporter capture sequence.

In some embodiments, the method comprises identifying a single engineered cell of the population of the engineered cells of which the number of mutations in the mutated stgRNA is higher than the mutation number determined in the stgRNA of the reference engineered cell.

In some embodiments, the method comprises identifying a engineered cell of the population of the engineered cells that is associated with a higher quantity of target antigen than other engineered cells of the population of engineered cells. In some embodiments, the method comprises identifying an engineered cell of the population of the engineered cells that is associated with a higher quantity of target antigen than a reference engineered cell, e.g., a reference engineered cell expressing the antigen-binding molecule of interest but not expressing an SHM enzyme under control of an immune-inducible promoter. In some embodiments, the method comprises determining a sequence of an antigen-binding molecule expressed by the identified engineered cell.

The quantity of such antigen associated with a cell and therefore an antigen-binding molecule expressed by the cell can be facilitated by the respective reporter oligonucleotides coupled to the antigen, wherein a reporter oligonucleotide coupled to an antigen comprises a reporter barcode sequence that identifies the antigen coupled thereto. For example, the quantity of such antigen associated with a cell (and its expressed antigen-binding molecule) can be determined based on a quantity/number of antigen sequence reads and/or UMIs associated with the cell (and its expressed antigen-binding molecule. Antigen sequence reads and/or UMIs can be associated bioinformatically with antigen-binding molecule sequences via shared partition barcode sequences. For example, binding affinity and/or binding specificity of an antigen-binding molecule to a target antigen can be determined based on independent observations of quantity/number of UMIs associated with the antigen from one or more partitions, wherein each of the one or more partitions comprise a cell expressing the same antigen-binding molecule. For other example, binding affinity and/or binding specificity of an antigen-binding molecule to a target antigen can be determined based on independent observations of quantity/number of UMIs associated with the antigen from one or more partitions, wherein each of the one or more partitions comprise a cell expressing an antigen-binding molecule belonging to the same clonotype group.

In some embodiments, the method described herein further comprises selecting a cell with greater than about 2.5%, 5%, or 10% difference in sequence identity to a reference engineered cell. For example, in certain embodiments, the method comprises sequencing the B-cell receptor (BCR) sequence of an engineered cell and comparing to the sequences derived from a reference cell. In some embodiment, the method involves determining the ratio of BCR mutations to stgRNA mutations in an engineered cell expressing the antigen-binding molecule of interest, and comparing to the ratio derived from a reference cell. In other embodiments, the percentage difference and the ratio can be combined in selecting the engineered cell expressing the antigen-binding molecule of interest.

### B. Methods of Making an Antigen-Binding Molecule of Interest

Another aspect of the present disclosure relates to a method of making an antigen-binding molecule of interest. In some embodiments, the method comprises first identifying the antigen-binding molecule of interest by the methods provided herein, and then purifying the antigen-binding molecule from the identified single engineered cell expressing the antigen-binding molecule thereof. In some embodiments, the method comprises first identifying the antigen-binding molecule of interest by the methods provided herein, and then producing the selected antigen-binding molecule. In other embodiments, the method further comprises selecting the antigen-binding molecule of interest identified by the methods provided herein.

Methods of making (e.g., producing, generating) an antigen-binding molecule (e.g., an antibody or functional fragments thereof) are generally known in the art. In some embodiments, the methods of making an antigen-binding molecule of interest provided herein comprise (a) culturing a cell (e.g., an engineered cell) expressing an antigen-binding molecule of interest; and (b) isolating the antigen-binding molecule of interest the cultured cell. In brief, an exemplary workflow for the approaches described herein generally involves compartmentalizing, depositing, and/or partitioning of single engineered cells from a population of engineered cells into discrete partitions (e.g., compartments or chambers), where each partition maintains separation of its own contents from the contents of other partitions, which facilitates the analysis, characterization, and/or generation of antigen-binding molecules of interest derived from those engineered cells.

After cell lysis and reverse transcription of V_{H} and V_{L} mRNAs produced in the droplets, the complementary DNAs from each cell carry a unique barcode that allows cognate V_{H} and V_{L} pairs to be identified by nucleic acid sequencing, followed by gene synthesis, cloning, and production of selected recombinant antigen-binding molecules.

As discussed above, a skilled artisan in the art will understand that the term partition generally refers to a discrete space or volume that may be suitable to contain one or more cells, one or more species of features or compounds, or to conduct one or more reactions. In some embodiments, the partitions are physical compartments such as, droplets, flow cells, reactions chambers, or wells (e.g., microwells). In some embodiments, the partitions of the disclosures are droplets. In some embodiments, the droplets can be a first phase (e.g., aqueous phase) in a second phase (e.g., oil) immiscible with the first phase. In some embodiments, the droplets can be a first phase in a second phase that does not phase separate from the first phase, such as, for example, a capsule (e.g., microcapsule) or liposome in an aqueous phase. In some embodiments, a partition of the disclosure can optionally include one or more other partitions, e.g., inner partitions or sub-partitions. Additional information regarding methods and systems for partitioning individual cells or cell populations in a plurality of partitions can be found in, for example, PCT Publication No. WO2018075693A1.

In some embodiments, the methods further include partitioning cell lysis reagents into the partition in order to release the contents of the partitioned single engineered cell (e.g., an engineered B cell). In some embodiments, the methods further include using the one or more nucleic acid barcode molecules and one or more nucleic acid analytes of the partitioned B single cell to generate one or more barcoded nucleic acid molecule comprising a coding sequence for an antibody produced by the partitioned B single cell, or any fragments thereof such as a B cell receptor heavy chain (VH), B cell receptor light chain (V_{L}), or any fragment thereof, e.g., variable regions including VDJ or VJ regions, constant regions, transmembrane regions, fragments thereof, and combinations of fragments thereof.

Once the single engineered cell (e.g., an engineered B cell)s are partitioned in individual droplets, nucleic acid sequencing can be used to determine nucleic acid sequences that encode one or more antibodies produced by the partitioned single engineered cell (e.g., an engineered B cell).

### Production of Recombinant Antigen-Binding Molecules

In some embodiments, the method further includes generating a recombinant antigen-binding molecule (e.g., an antibody or a functional fragment thereof) using the determined nucleic acid sequences of the partitioned engineered cell (e.g., a partitioned B cell). One skilled in the art will appreciate that the determined nucleic acid sequences of the V_{H} and V_{L} mRNAs can be used to construct a full-length gene encoding a desired recombinant antigen-binding molecule. For example, a DNA oligomer containing a full-length nucleotide sequence coding for a given V_{H} and V_{L} domain of the desired antigen-binding molecule can be synthesized. In addition or alternatively, several small oligonucleotides coding for portions of the desired recombinant antigen-binding molecule (e.g., an antibody or a functional fragment thereof) can be synthesized and then ligated. The individual oligonucleotides generally contain 5′ or 3′ overhangs for complementary assembly.

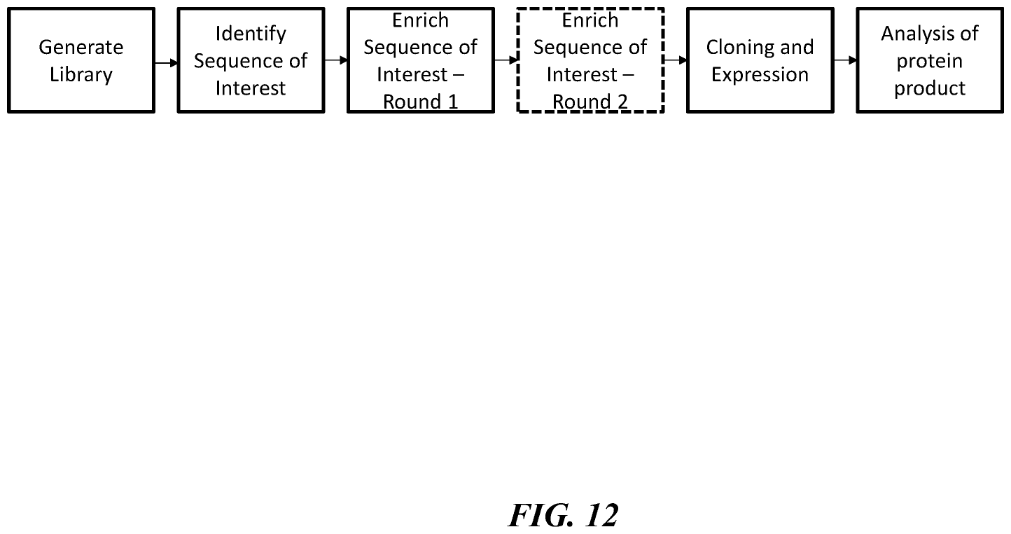

A general workflow of methods provided herein is provided in FIG. 12. Briefly, (1) a library can be generated (e.g., a barcoded library of sequences of a single cell immune repertoire of a subject); (2) antigen-binding molecule sequences of interest can be identified (e.g., a V(D)J sequence, such as BCR or antibody (e.g., heavy/light chain) sequences), for example by sequencing; (3) the sequence(s) of interest can be enriched from the library (e.g., by using 1 or 2 rounds of PCR); (4) cloning (and optionally expressing) the enriched sequence(s) of interest into an appropriate expression vector; and (5) optionally analyzing the protein (e.g., antibody) encoded by the sequence of interest.

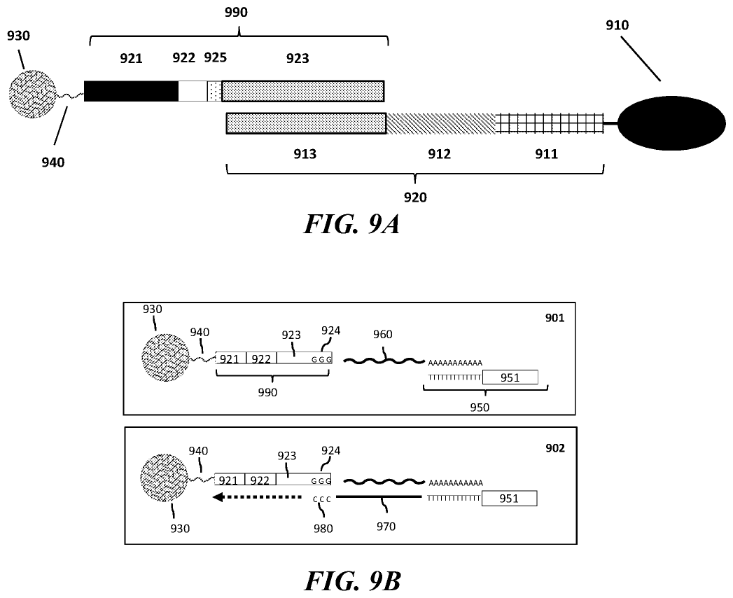

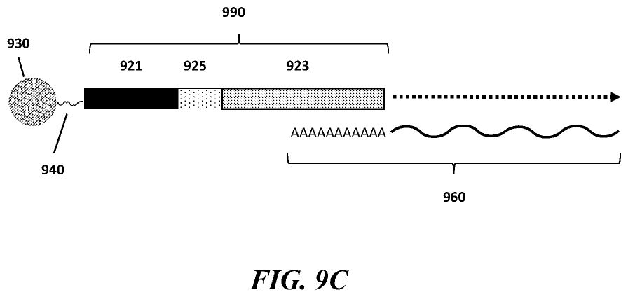

In some instances, a barcoded nucleic acid library comprising antigen-binding molecules is generated as described herein. See, e.g., FIGS. 9B and 9C.

Molecules of the library can have the structure, from 5′ to 3′, of identification sequence to coding sequence. For example, molecules of the library can have the structure, from 5′ to 3′, of: (1) barcode sequence; (2) unique molecular identifier sequence; (3) template switch oligonucleotide sequence (4) variable sequence of an antigen-binding molecule (e.g., V(D)J sequence, as provided herein); and optionally (5) constant sequence of the antigen-binding molecule. In some embodiments, one or more adapter sequences (such as sequencing platform specific sequences, such as a sequencing primer or primer binding sequence, e.g., an Illumina R1 or R2) can be located either 5′ or 3′ to the sequence of a molecule of a library, or both.

In some instances, a barcoded gene expression library is generated (e.g., from single cells as described herein) from a plurality of cells comprising an immune molecule, such as a BCR or antibody. The barcoded library can then be sequenced and analyzed to identify paired antigen-binding molecule sequences from single cells (e.g., comprising a common barcode sequence), such as paired BCRs (light/heavy chain sequences) and paired antibody sequences (light/heavy chain sequences). Sequences of antigen-binding molecules of interest (e.g., paired light/heavy chain antibodies) can then be directly enriched (e.g., amplified) from the barcoded library for subsequent processing and analysis in, e.g., an expression vector. In some instances, primers are designed to amplify paired antigen-binding molecule sequences, e.g., light and heavy chain antibody sequences, from the library for cloning into one or more suitable expression vectors.

Enrichment of a nucleic acid sequence of interest from, e.g., a barcoded gene expression library, can allow expedited isolation of the nucleic acid, and the expression and/or analysis for the amino acid sequence which it encodes. For example, enrichment (e.g., using one or more PCR reactions) of sequences of interest (e.g., a V(D)J sequence, such as BCR or antibody (e.g., heavy/light chain) sequences) and direct cloning of those enriched sequences (such as a light and heavy chain sequence of an antibody) into an appropriate expression vector can be utilized to avoid costly and time consuming methodologies (such as gene synthesis) employed to generate an expression vector configured to express immune molecules (e.g., antibodies) of interest. A nucleic acid sequence of interest can be enriched by amplifying the nucleic acid sequence of interest based on an identification sequence (e.g., barcode or UMI) associated with the nucleic acid sequence of interest, for example, by using a scheme such as is illustrated in FIG. 12. In some embodiments, a nested amplification approach can be employed to further enrich the nucleic acid sequence of interest.

As part of an enrichment protocol, a nucleic acid primer can be designed that anneals to one or more identification sequences in a molecule that harbors a nucleic acid sequence of interest, e.g., a barcode sequence or unique molecule identifier. Another primer can be designed to anneal to a sequence downstream of the identification sequence, and can be configured such that the nucleic acid sequence can be amplified using the primers, e.g., by polymerase chain reaction. A second round of amplification can be performed using a different set of primers to further enrich the nucleic acid sequence of interest.

After enriching a nucleic acid sequence of interest, it can be cloned into a vector and subsequently expressed in an expression system. Such cloning and expression can yield protein for analysis. For example, a candidate B cell receptor, or antibody or antigen-binding fragment thereof can be expressed in an expression system where such a nucleic acid sequence of interest is cloned. Such a protein can be a therapeutic candidate, a gene of interest, a protein variant of interest, or another protein to be analyzed. In some instances, primers are designed to amplify paired immune molecule sequences from single cells (e.g., comprising a common barcode sequence), such as paired BCRs (light/heavy chain sequences) and paired antibody sequences (light/heavy chain sequences). These amplified, paired immune molecule sequences (e.g., paired light and heavy chain antibody sequences) can then be optionally processed for subsequent cloning into an expression vectors for expression of functional immune molecules (e.g., a plasmid configured to co-express paired immune molecule subunits, such as an antibody heavy and light chain).

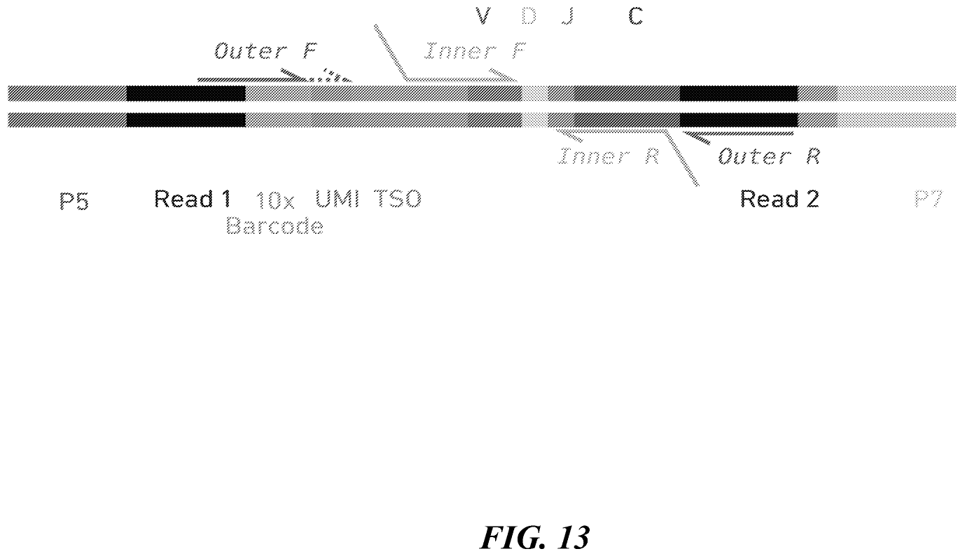

In some embodiments, a double (e.g., nested) PCR strategy is employed for the enrichment of a nucleic acid sequence of interest. An example of a nested PCR scheme is illustrated in FIG. 13. In this example, a cDNA molecule of interest is illustrated which comprises a first read sequence, a barcode sequence, a unique molecular identifier sequence (UMI), a template switch oligonucleotide (TSO), a V sequence, a D sequence, a J sequence, a constant (C) sequence, and a second read sequence. Primers can be designed to enrich the sequence for an antibody (i.e., the V, D, J, and C sequences) using a double PCR strategy. Such a double PCR strategy can employ a first enrichment step and a second enrichment step. The outer F (forward) and outer R (reverse) primers can be primers employed for a first PCR enrichment step to enrich one of a plurality of cDNA molecules comprising barcodes. The outer F primer can comprise a sequence complementary to the barcode, and the outer R primer can comprise a sequence complementary to the complement of the second read sequence. The inner F (forward) and inner R (reverse) primers can be employed for a second enrichment step to further enrich the product of the first enrichment step. The inner F primer can be complementary to the V sequence, and optionally part of the TSO sequence. The inner R primer can be complementary to the C sequence and the J sequence. In some cases, the inner F and inner R primers can include non-binding handles that can allow cloning into a vector or enable pairing of sequences, for example using overlap extension.

Methods and systems for enriching and cloning antigen-binding molecules are briefly described herein.

### Enrichment of Recombinant Antigen-Binding Molecules

A nucleic acid sequence of interest can be enriched from a library of nucleic acid molecules or sequences. The library of nucleic acid molecules or sequences may have been derived from a cell(s), or sample(s) of cell(s) of a donor, (e.g., a human donor), or an engineered cell expressing a variant antibody, or variant antigen-binding fragment thereof. The nucleic acid sequence of interest may be a nucleic acid sequence encoding a selected antibody, or selected antigen-binding fragment thereof, and the enrichment may be an error-prone amplification reaction that produces polynucleotides encoding variant antibodies or variant antigen-binding fragments thereof. Methods for enriching a nucleic acid sequence of interest can comprise an amplification reaction. Examples of amplification reactions can include linear amplification, polymerase chain reaction (PCR), and nested PCR In some embodiments, a different amplification reaction can be employed.

PCR can comprise denaturation, annealing, and extension steps. Denaturation can comprise exposing the nucleic acid to a temperature capable of melting the nucleic acid. In some cases, denaturation can occur between 94° C. and 98° C. In some cases, denaturation can occur at 94° C., 95° C., 96° C., 97° C., or 98° C. Denaturation can last for at least 15 seconds, at least 30 seconds, at least 45 seconds, at least 60 seconds, at least 75 seconds, at least 90 seconds, at least 105 seconds, at least 120 seconds, at least 135 seconds, at least 150 seconds, at least 165 seconds, or at least 180 seconds. Annealing can comprise exposing the melted nucleic acid to a temperature which can allow the binding of a primer to the nucleic acid. In some cases, annealing can occur between 50° C. and 75° C. In some cases, annealing can occur between 55° C. and 70° C. In some instances, annealing can occur at 55° C., 56° C., 57° C., 58° C., 59° C., 60° C., 61° C., 62° C., 63° C., 64° C., 65° C., 66° C., 67° C., 68° C., 69° C., or 70° C. Annealing can last for at least 15 seconds, at least 30 seconds, at least 45 seconds, at least 60 seconds, at least 75 seconds, at least 90 seconds, at least 105 seconds, at least 120 seconds, at least 135 seconds, at least 150 seconds, at least 165 seconds, or at least 180 seconds. Extension can comprise exposing the nucleic acid to a temperature at which extension can occur, thereby amplifying the nucleic acid, for example by a polymerase present in the partition with the nucleic acid. Extension can occur between 65° C. and 75° C. In some cases, extension can occur at 65° C., 66° C., 67° C., 68° C., 69° C., 70° C., 71° C., 72° C., 73° C., 74° C., or 75° C. The steps of denaturation, annealing, and extension can be repeated for a number of cycles. In some cases, PCR cycling can proceed for at least 1 cycle. In some cases, PCR cycling can proceed for at least 5, 10, 15, 20, 25, 30, 35, or 40 cycles. In some cases, PCR cycling can proceed for between 1 cycle and 40 cycles, between 1 cycle and 35 cycles, between 1 cycle and 30 cycles, between 1 cycle and 25 cycles, between 1 cycle and 20 cycles, between 1 cycle and 15 cycles, between 1 cycle and 10 cycles, between 1 cycle and 5 cycles, between 5 cycles and 40 cycles, between 5 cycles and 35 cycles, between 5 cycles and 30 cycles, between 5 cycles and 25 cycles, between 5 cycles and 20 cycles, between 5 cycles and 15 cycles, between 5 cycles and 10 cycles, between 10 cycles and 40 cycles, between 10 cycles and 35 cycles, between 10 cycles and 30 cycles, between 10 cycles and 25 cycles, between 10 cycles and 20 cycles, between 10 cycles and 15 cycles, between 15 cycles and 40 cycles, between 15 cycles and 35 cycles, between 15 cycles and 30 cycles, between 15 cycles and 25 cycles, between 15 cycles and 20 cycles, between 20 cycles and 40 cycles, between 20 cycles and 35 cycles, between 20 cycles and 30 cycles, between 20 cycles and 25 cycles, between 25 cycles and 40 cycles, between 25 cycles and 35 cycles, between 25 cycles and 30 cycles, between 30 cycles and 40 cycles, between 30 cycles and 35 cycles, or between 35 cycles and 40 cycles.

Methods for enriching a nucleic acid sequence of interest, such as an amplification reaction, can comprise contacting the nucleic acid sequence of interest with PCR reaction, e.g., with reagents for a PCT reaction. In some cases, reagents for a PCR reaction can comprise a polymerase, one or more sets primers, and a dNTP mixture. In some cases, a polymerase can be a DNA polymerase, an RNA polymerase, or a reverse transcriptase. In some cases, a set of primers can comprise at least 2 primers which can be complementary to a region of a nucleic acid of interest, such that the region of the nucleic acid of interest can be amplified via PCR using the primer pair. In some cases, a partition can comprise at least 1, 2, 3, 4, 5, 6, 7, 8, 9, 10, 11, 12, 13, 14, 15, 16, 17, 18, 19, or 10 sets of two primers. In some cases, a partition can comprise no more than 1, 2, 3, 4, 5, 6, 7, 8, 9, 10, 11, 12, 13, 14, 15, 16, 17, 18, 19, or 10 sets of two primers. In some cases, a partition can comprise one or more probes. A probe can be a DNA binding dye, a hydrolysis probe, a molecular beacon, a dual hybridization probe, an eclipse probe, or an amplifluor probe. In some cases, a probe can be a SYBR green probe, a Taqman probe, a Scorpions PCR primer, a LUX PCR primer, or a QZyme PCR primer. In some cases, a probe can comprise a label, which can be colored, opaque, radiopaque, fluorescent, radioactive, or otherwise detectable. In some cases, a partition can comprise additional reagents, which can comprise magnesium, salt, glycerol, buffer, dye, or other reagents. A first set of partitions and a second set of partitions can be obtained. In some cases, these partitions can each comprise a nucleic acid molecule, e.g., a target nucleic acid molecule, which can be amplified and detected. A set of partitions can comprise a plurality of partitions. In some cases, a set of partitions can comprise at least 1, at least 10, at least 100, at least 1,000, at least 10,000, at least 100,000, at least 1,000,000, or at least 10,000,000 partitions. In some cases, a set of partitions can comprise a set of droplets, e.g., an aqueous droplet in an emulsion. For example, a first set of partitions can comprise a first set of droplets, and a second set of partitions can comprise a second set of droplets.

Methods for enriching a nucleic acid sequence of interest, such as an amplification reaction, can comprise contacting the nucleic acid sequence of interest with a nucleic acid primer. In some cases, a nucleic acid primer can be an oligonucleotide suitable for a PCR reaction (e.g., a PCR primer).

A nucleic acid primer can comprise an oligonucleotide. An oligonucleotide can be a molecule which can be a chain of nucleotides. Oligonucleotides described herein can comprise ribonucleic acids. Oligonucleotides described herein can comprise deoxyribonucleic acids. In some cases, oligonucleotides can be of any sequence, including a user-specified sequence.

In some embodiments, an oligonucleotide can comprise G, A, T, U, C, or bases that are capable of base pairing reliably with a complementary nucleotide. 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine or 2-thio-uridine are examples of such bases, although many others are known. An oligonucleotide can comprise an LNA, a PNA, a UNA, or an morpholino oligomer, for example. The oligonucleotides used herein may contain natural or non-natural nucleotides or linkages.

An oligonucleotide can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some cases, an oligonucleotide can be between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some cases, an oligonucleotide can be no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

In some cases, an oligonucleotide can be wholly single stranded. In some cases, an oligonucleotide can be partially double stranded. A partially double stranded region can be at the 3′ end of the oligonucleotide, at the 5′ end of the oligonucleotide, or between the 5′ end and 3′ end of the oligonucleotide. In some cases, there may be more than one double stranded region.

Methods can comprise using a nucleic acid primer complementary to a portion of the nucleic acid sequence of interest (e.g., the identification sequence or a portion thereof). In some embodiments, a nucleic acid primer complementary to at least a portion of the identification sequence of a nucleic acid sequence of interest can be complimentary to at least a portion of a barcode sequence, at least a portion of a template switch oligonucleotide sequence, at least a portion of a unique molecular identifier sequence, or a combination thereof. In some embodiments, a nucleic acid primer can be complementary to a barcode sequence and a read sequence of the nucleic acid sequence of interest, or a portion thereof. In some embodiments, a nucleic acid primer can be complementary to a barcode sequence and a unique molecular identifier sequence, or a portion thereof. This can be done to amplify the nucleic acid sequence of interest, for example using an amplification reaction provided herein (e.g., PCR or nested PCR).

In some embodiments, a nucleic acid primer can further comprise a nucleic acid sequence that can be complementary to at least a portion of a coding sequence of the nucleic acid sequence of interest. In some embodiments, the nucleic acid primer can comprise a nucleic acid sequence that can be complementary to a variable region of the nucleic acid sequence of interest, such as the variable region of a B cell receptor, or the variable region of an antigen or antibody binding fragment thereof. As an example, the nucleic acid primer can comprise a nucleic acid sequence that is complementary to a V(D)J sequence or a portion thereof, a V sequence of the V(D)J sequence or a portion thereof, a D sequence of the V(D)J sequence or a portion thereof, or a J sequence of the V(D)J sequence or a portion thereof. In some embodiments, a nucleic acid primer can be complementary to a portion of the variable region of the nucleic acid sequence of interest that is different than that of a different nucleic acid molecule.

In some embodiments, a nucleic acid primer can further comprise a non-binding handle. A non-binding handle can be a nucleic acid sequence on the nucleic acid primer that is not complementary to a segment of the nucleic acid sequence of interest. In some embodiments, a non-binding handle may not bind any nucleic acid sequence in the plurality of nucleic acid molecules. In some embodiments, a non-binding handle can be utilized for cloning into recipient vector or to enable pairing of specific heavy/light (or TRA/TRB) sequences using overlap extension or a similar method after enrichment of the nucleic acid molecule.

In some embodiments, methods provided herein can further comprise using another nucleic acid primer to amplify said nucleic acid sequence of interest, wherein said another nucleic acid primer is different from said nucleic acid primer. For example, a method can comprise using a first nucleic acid primer and a second nucleic acid primer (e.g., a forward primer and a reverse primer for a PCR reaction).

In some embodiments, the another nucleic acid primer can comprise a non-binding handle. Such a non-binding handle can be a nucleic acid sequence on the nucleic acid primer that is not complementary to a segment of the nucleic acid sequence of interest. In some embodiments, a non-binding handle may not bind any nucleic acid sequence in the plurality of nucleic acid molecules. In some embodiments, a non-binding handle can be utilized for cloning into recipient vector or to enable pairing of specific heavy/light (or TRA/TRB) sequences using overlap extension or a similar method after enrichment of the nucleic acid molecule.

In some methods, the nucleic acid primer and another (e.g., a second) nucleic acid primer (e.g., a forward primer and a reverse primer) can be configured to anneal to sequences flanking at least a portion of said nucleic acid sequence of interest. For example, a nucleic acid primer can be configured to anneal to a sequence upstream of the nucleic acid sequence of interest, and a second nucleic acid primer can be configured to anneal to a complement of a sequence downstream of the nucleic acid sequence of interest. In some embodiments, two such nucleic acid primers can be configured to yield a copy of the nucleic acid sequence of interest after an amplification reaction such as PCR.

In some embodiments, a second nucleic acid primer can comprise a nucleic acid sequence complementary to a binding sequence on the nucleic acid sequence of interest, or a complement thereof. Such a binding sequence can be on a coding section of the nucleic acid sequence of interest, or upstream or downstream of the coding section of the nucleic acid sequence of interest. In some embodiments, the second nucleic acid primer can be complementary to at least a portion of a nucleic acid sequence coding for a constant region of an amino acid sequence coded for by the nucleic acid sequence of interest, such as a B cell receptor, or an antibody or antigen-binding fragment thereof, or a complement of such a nucleic acid sequence.

The second nucleic acid primer can be at least partially complementary to a variable region of a nucleic acid sequence of interest (e.g., a V(D)J sequence, a V sequence, a D sequence, or a J sequence). For example, the second nucleic acid primer can be further complementary to at least a portion of a nucleic acid sequence coding for a J region of an antibody or antigen-binding fragment thereof or at least a portion of a nucleotide sequence coding for framework 4.

Methods can further comprise a second enrichment step, such as a second amplification reaction. A second amplification reaction can comprise linear amplification, PCR, or another amplification scheme. In some embodiments, a second PCR reaction can be implemented to enrich or further enrich a nucleic acid sequence of the plurality of nucleic acid molecules. As an example, a nested PCR scheme can be utilized to provide enrichment of a nucleic acid sequence of interest.

A second round of amplification can comprise contacting the nucleic acid sequence with a third primer and a fourth primer. A third primer or a fourth primer can comprise an oligonucleotide as provided herein. A third primer and a fourth primer can be configured to specifically enrich the nucleic acid sequence. In some embodiments, the third primer can be different than the first primer, or the fourth primer can be different than the second primer.

The third primer can be complementary to at least a portion of the identification sequence. In some embodiments, the third primer can be complementary to a portion of a barcode of the identification sequence. In some cases, the third primer can be complementary to a 5′ end of a barcode of the identification sequence.

In some embodiments, a third primer can be complementary to a portion of the identification sequence upstream of a barcode of the identification sequence. For example, a third primer can be complementary to at least a portion of a read sequence of a nucleic acid molecule. In some embodiments, a third primer can be complementary to at least a portion of a variable sequence, such as a nucleic acid sequence coding for a V(D)J sequence.

A fourth primer can be complementary to the complement of another segment of the nucleic acid molecule, such that the nucleic acid sequence of interest can be flanked by the third primer and the fourth primer. In some embodiments, the fourth primer can be complementary to a nucleic acid sequence downstream of a coding sequence of the nucleic acid sequence of interest. In some embodiments, the fourth primer can be complementary to at least a portion of the complement of a constant region, or framework 4 of the nucleic acid sequence of interest.

In some methods, enrichment may be performed using first and second primers in which: (i) the first primer is complementary to a cell barcode and UMI sequence and the second primer is complementary to a nucleic acid sequence encoding at least a portion of the J region of the antibody, or antigen-binding fragment thereof (or a complement thereof); (ii) the first primer is complementary to a leader sequence, and the second primer is complementary to nucleic acid sequences encoding the J region and CDR3 of the antibody, or antigen-binding fragment thereof, (or a complement thereof); (iii) the first primer is complementary to the cell barcode and UMI sequences and the second primer is complementary to a nucleic acid sequence encoding at least a portion of the framework 4 region of the variable region of the antibody, or antigen-binding fragment thereof, (or a complement thereof); or (iv) the first primer is complementary to a leader sequence and the second primer is complementary to nucleic acid sequences encoding at least portions of the framework 4 region and constant regions of the antibody, or antigen-binding fragment thereof, (or a complement thereof).

In some embodiments, embodiments in which nested amplification reactions are performed, the amplification reactions may be conducted such that: (i) a first amplification is performed with a first primer complementary to a cell barcode and UMI sequences and a second primer complementary to at least a portion of the coding sequence of the constant region of the antibody, (or a complement thereof), and a second amplification is performed with a third primer complementary to a coding sequence for the V(D)J region of the antibody, or antigen-binding fragment thereof, and a fourth primer complementary to at least a portion of the coding sequence downstream of the V(D)J region of the antibody, e.g., in the constant region, (or a complement thereof); (ii) a first amplification is performed with a first primer complementary to at least a portion of a barcode and a second primer complementary to a read sequence (or complement thereof), and a second amplification is performed with a third primer complementary to a coding sequence for the V region of the antibody, or antigen-binding fragment thereof, and a fourth primer complementary to at least a portion of a coding sequence downstream of the constant region and J region of the antibody, or antigen-binding fragment thereof, (or a complement thereof); (iii) a first amplification is performed with a first primer complementary to a cell barcode and UMI sequence and a second primer complementary to at least a portion of the coding sequence for the J region of the antibody, or antigen-binding fragment thereof, (or complement thereof), and a second amplification is performed with a third primer complementary to a leader sequence and a fourth primer complementary to at least a portion of the coding sequence for CDR3 of the antibody, or antigen-binding fragment thereof, (or a complement thereof); or (iv) a first amplification is performed with a first primer complementary to a cell barcode and UMI sequences and a second primer configured to be complementary to a sequence encoding framework 4 of the antibody, or antigen-binding fragment thereof, (or a complement thereof), and a second amplification is performed with a third primer complementary to a leader sequence and a fourth primer complementary to at least a portion of the coding sequence of the antibody's framework 4 and constant regions, (or a complement thereof).

In some methods, others of the plurality of nucleic acid molecules can be not amplified. For example, nucleic acid molecules that do not comprise the nucleic acid sequence of interest can be not amplified. In some methods, others of the plurality of nucleic acid molecules can be amplified by less than a threshold amount. For example, a nucleic acid sequence of interest can be amplified by more than 100 times, more than 1000 times, more than 10,000 times, more than 100,000 times, more than 1,000,000 times, or more than 10,000,000 times more than others of the plurality of nucleic acid molecules.

Methods provided herein can further comprise determining an enrichment level of the nucleic acid sequence of interest. Enrichment can be determined, for example, by fluorescence, gel electrophoresis, sequencing, or another acceptable method for determining enrichment.

A nucleic acid sequence of interest can be enriched by a factor of at least 1000 at least 10,000, at least 100,000, at least 1,000,000, or at least 10,000,000. In some embodiments, a nucleic acid sequence of interest can be enriched by a factor sufficient for cloning the nucleic acid sequence of interest. In some embodiments, a nucleic acid sequence of interest can be further enriched by a second amplification step by a factor of at least 1000, at least 10,000, at least 100,000, at least 1,000,000, or at least 10,000,000.

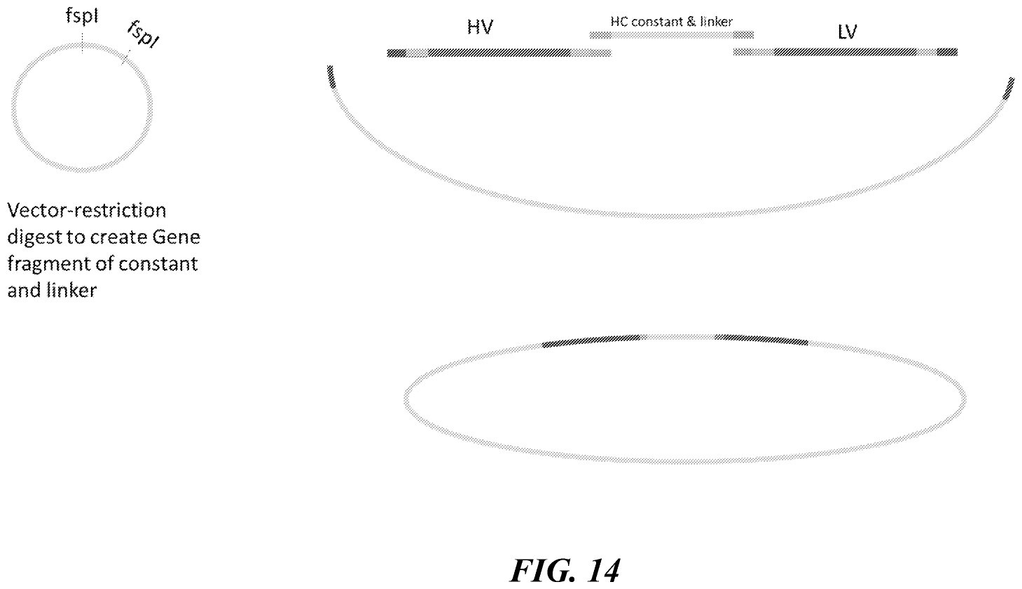

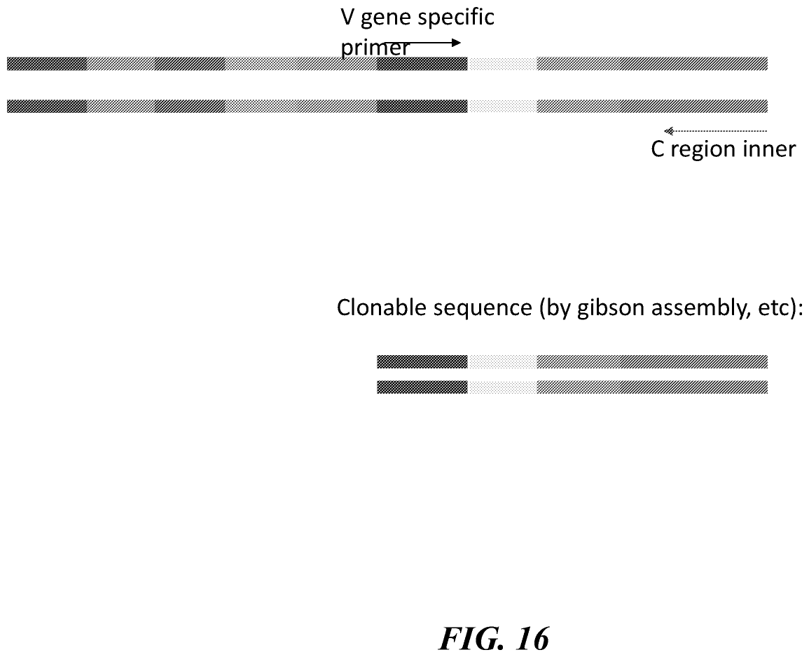

Also provided herein are methods comprising enriching a nucleic acid sequence of interest based on at least a portion of a constant region of said nucleic acid sequence of interest. This enrichment can yield an enriched nucleic acid sequence of interest. In some embodiments, the method can further comprise modification of the enriched nucleic acid sequence yielding a modified enriched nucleic acid sequence. A pictorial outline of such a method is provided in FIG. 16. The modified enriched nucleic acid sequence can be compatible with a vector. For example, the modified enriched nucleic acid sequence can have a structure (e.g., a nucleic acid sequence) that can be directly incorporated into a vector. A vector can be a vector suitable for cloning or expression of the modified enriched nucleic acid sequence or other use of the modified enriched nucleic acid sequence. A vector can be any vector described herein. A pictorial outline of a nucleic acid sequence that is compatible with a vector (including incorporation of the nucleic acid sequence into the vector) is provided in FIG. 14.

A nucleic acid sequence of interest can be a nucleic acid sequence described herein. For example, a nucleic acid sequence of interest can code for at least a portion of a cell surface protein of a cell, such as a B cell receptor (or fragment thereof). A nucleic acid sequence of interest can comprise a constant region. In some embodiments, the nucleic acid sequence of interest can comprise a sequence encoding a V(D)J sequence or a portion thereof, such as a V sequence (or portion thereof), a D sequence (or portion thereof), or a J sequence (or portion thereof), as described herein. In some embodiments, the constant region of a nucleic acid sequence of interest can comprise a sequence encoding a V(D)J sequence or a portion thereof, such as a V sequence (or portion thereof), a D sequence (or portion thereof), or a J sequence (or portion thereof), as described herein. In some embodiments, a nucleic acid sequence of interest can comprise a barcode (e.g., as provided herein), a UMI (e.g., as provided herein), or a 5′ untranslated region (5′ UTR) of a gene of interest (e.g., a BCR gene). In some embodiments, the nucleic acid sequence of interest can comprise complementary deoxyribonucleic acid (cDNA) of an RNA transcript of interest (e.g., a BCR transcript).

Enriching can be performed using a first nucleic acid primer. A first nucleic acid primer can be complementary to a region of the nucleic acid sequence of interest. For example, the first nucleic acid primer can be complementary at least to a barcode or portion thereof on said nucleic acid sequence of interest. In some embodiments, the first nucleic acid primer can be complementary to a UMI sequence or a portion thereof on said nucleic acid sequence of interest. In some embodiments, the first nucleic acid primer can be complementary at least to a 5′ untranslated region (5′ UTR) or a portion thereof on said nucleic acid sequence of interest. In some embodiments, the first nucleic acid primer can be a framework leader (FWR1) primer.

Enriching can be performed using a second nucleic acid primer. In some embodiments, the second nucleic acid primer can be used with the first nucleic acid primer to enrich the nucleic acid sequence of interest. In some embodiments, the second nucleic acid primer can be complementary at least to a constant region or portion thereof on said nucleic acid sequence of interest. In some embodiments, the second nucleic acid primer can be complementary at least to a V(D)J sequence or portion thereof on said nucleic acid sequence of interest. In some embodiments, the second nucleic acid primer can be complementary at least to a J sequence or portion thereof on said nucleic acid sequence of interest. In some embodiments, the second nucleic acid primer can be complementary at least to a nucleic acid sequence of a junction region or portion thereof on said nucleic acid sequence of interest.

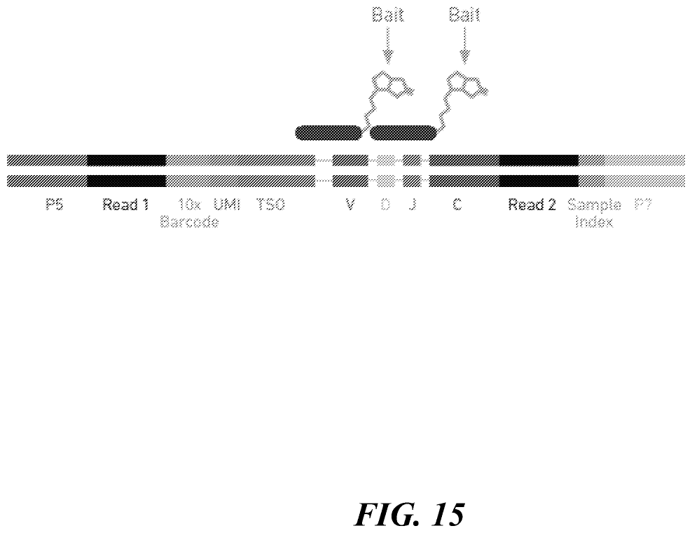

Enriching can be performed using hybridization capture. In some embodiments, the hybridization capture can be based on hybridization of a nucleic acid probe to a sequence on said nucleic acid sequence of interest such as a constant sequence or a junction sequence. For example, a probe can hybridize to a portion of a junction sequence such as a V(D)J sequence or a portion thereof, such as a V sequence or a portion thereof, a D sequence or a portion thereof, or a J sequence or a portion thereof. In some embodiments, a probe can hybridize to a V sequence and a D sequence (or a portion thereof) or a D sequence and a J sequence (or a portion thereof). The probe may comprise a functional group (such as a biotin molecule) to enable purification of the hybridized target nucleic acid molecule (e.g., using streptavidin conjugated beads, such as magnetic beads). See, e.g., FIG. 15.

A nucleic acid primer used for enriching can be selected based on Rapid Amplification of cDNA Ends (RACE) sequencing. RACE sequencing can be a technique used to obtain a sequence (e.g., 5′ RACE) of a nucleic acid (e.g., an RNA transcript), such as a nucleic acid (e.g., an RNA transcript) found within a cell. RACE sequencing can result in the production of a cDNA copy of a sequence of interest, produced through reverse transcription, followed by PCR amplification of the cDNA copies (see RT-PCR). The amplified cDNA copies can be sequenced and can map to a unique genomic region. In some embodiments, the RACE-products can be sequenced by next generation sequencing technologies.

In some embodiments, a method can further comprise cloning a modified enriched nucleic acid into a vector, such as a vector the modified enriched nucleic acid sequence is compatible with. Cloning can be performed using any acceptable method, including methods provided herein (e.g., in the cloning section).

Nucleic acid primers should not be interpreted to be specific to a particular nucleic acid strand. For example, in some embodiments, a first nucleic acid molecule can be complementary to a complement of an identification sequence as described herein. In some embodiments, a second nucleic acid molecule can be complementary to a binding sequence as designed herein.

### Modification of an Enriched Nucleic Acid Sequence of Interest

Further modification of a nucleic acid sequence of interest can be performed, for example after the nucleic acid sequence of interest has been enriched. In some embodiments, modification of a nucleic acid sequence of interest can be performed in preparation for analysis of the nucleic acid sequence of interest, to analyze the nucleic acid sequence of interest, or to prepare the nucleic acid sequence of interest for cloning.

Methods can further comprise performing fragmentation of a nucleic acid sequence of interest. Nucleic acid fragmentation (e.g., footprinting), such as by OH radicals, can be a tool to probe nucleic acid structure and nucleic acid-protein interactions. Such methods can provide structural information with single base pair resolution. Footprinting can refer to assays in which either the binding of a ligand to a specific sequence of bases or the conformation of the nucleic acid inhibits nicking of the phosphodiester backbone of nucleic acid polymer by a reagent. Intimate interactions between proteins and nucleic acids can be widely examined by footprinting methods. A prerequisite of such assays can be the ability to produce and detect high-quality nucleic acid fragmentation around the protein-protected areas. Nucleic acid fragmentation can be achieved by using a variety of enzymatic and chemical reagents. This can be highly related to the development of chemical hydroxyl radical footprinting using Fenton chemistry and peroxonitrous acid. Hydroxyl radicals can engender breaks of the phosphodiester backbone in a non-specific sequence manner and, hence, can be utilized for footprinting assays. Using hydroxyl radical methods over enzymatic footprinting can be advantageous because it can provide great sensitivity to nucleic acid structures, such as sequence-dependent curvature and RNA folding.

Methods can further comprise A-tailing of a nucleic acid sequence of interest. A-tailing can comprise an enzymatic method for adding a non-templated nucleotide to the 3′ end of a blunt, double-stranded DNA molecule. A-tailing can be performed to prepare a T-vector for use in TA cloning or to A-tail a PCR product produced by a high-fidelity polymerase (e.g., other than Taq) for use in TA cloning. TA cloning can be a rapid method of cloning PCR products that can utilize stabilization of the single-base extension (adenosine) produced by Taq polymerase by the complementary T (thymidine) of the T-vector prior to ligation and transformation. This technique may not utilize restriction enzymes and PCR products can be used directly without modification. Additionally, in some embodiments PCR primers do not need to be designed with restriction sites, making the process less complicated. In some embodiments, A-tailing can be non-directional, meaning the insert can go into the vector in both orientations.

Methods can further comprise performing a sample index polymerase chain reaction (SI-PCR) on a nucleic acid sequence of interest. SI-PCR can utilize different pairs of index primers on a nucleic acid molecule. In some cases, index primers can be added to individual samples in a second thermocycling step, for example after initial amplification of the target region. This can allow mixing of many samples together (e.g., up to 96) and simultaneous sequencing of the samples. Following sequencing, for example on an Illumina MiSeq, software can be able to identify these indexes on each sequence read, in some cases allowing separation of the reads for each different nucleic acid molecule.

Methods can further comprise V(D)J enrichment of a nucleic acid sequence of interest. This can be accomplished, for example, using PCR or another amplification method to amplify a V(D)J sequence or a fragment thereof from the enriched nucleic acid sequence of interest.

Modification of a nucleic acid sequence of interest or enriched nucleic acid sequence of interest can comprise addition of Gibson ends (e.g., Gibson Assembly) to said amplified nucleic acid sequences. Addition of Gibson ends (e.g., Gibson Assembly) can allow cloning or joining of two nucleic acid sequences without restriction sites. In some cases, addition of Gibson ends can allow joining of any two fragments regardless of sequence. Gibson assembly can be performed in a manner to leave no scar between joined nucleic acid sequence. Gibson assembly can be used to combine a plurality (e.g., 2, 3, 4, 5, 6, 7, 8, 9, 10, or more) of fragments. Gibson assembly can be performed, for example, as described in Gibson D G, Young L, Chuang R Y, Venter J C, Hutchison C A 3rd, Smith H O. Enzymatic assembly of DNA molecules up to several hundred kilobases. Nat Methods. 2009; 6(5):343-345. doi:10.1038/nmeth.1318

Gibson assembly can simultaneously combine a plurality of DNA fragments, e.g., based on sequence identity. The DNA fragments contain an about 20-40 base pair overlap with adjacent DNA fragments. These DNA fragments can be mixed with one or more enzymes (e.g., a cocktail of 3 enzymes), along with other buffer components. In some embodiments, the enzymes can include an exonuclease, a DNA polymerase, and a DNA ligase.

During Gibson assembly, modification of a nucleic acid sequence of interest or enriched nucleic acid sequence of interest can comprise combining a second nucleic acid of interest with the nucleic acid of interest or enriched nucleic acid of interest. In some embodiments, the second nucleic acid sequence of interest can be enriched. Such combining can comprise, for example, using one or more overlap extension primers to link the nucleic acid sequence of interest or enriched nucleic acid sequence of interest to the second nucleic acid sequence of interest. In some cases, such case can comprise using a nucleic acid linker to join the second nucleic acid sequence of interest to the nucleic acid sequence of interest or the enriched nucleic acid sequence of interest.

During Gibson assembly, a second nucleic acid sequence of interest can be a nucleic acid sequence described herein. For example, a second nucleic acid sequence of interest can code for at least a portion of a cell surface protein of a cell, such as a B cell receptor (or fragment thereof). In some cases, an enriched nucleic acid sequence of interest can comprise first chain of a B cell receptor (or fragment thereof), while a second nucleic acid sequence of interest can comprise a second chain of a B cell receptor (or fragment thereof). A second nucleic acid sequence of interest can comprise a constant region. In some embodiments, the second nucleic acid sequence of interest can comprise a sequence coding for a V(D)J sequence or a portion thereof, such as a V sequence (or portion thereof), a D sequence (or portion thereof), or a J sequence (or portion thereof), as described herein. In some embodiments, the constant region of a second nucleic acid sequence of interest can comprise a sequence coding for a V(D)J sequence or a portion thereof, such as a V sequence (or portion thereof), a D sequence (or portion thereof), or a J sequence (or portion thereof), as described herein. In some embodiments, a second nucleic acid sequence of interest can comprise a barcode (e.g., as provided herein), a UMI (e.g., as provided herein), or a 5′ untranslated region (5′ UTR). In some embodiments, the second nucleic acid sequence of interest can comprise complementary deoxyribonucleic acid (cDNA).

During Gibson assembly, the exonuclease can chew back DNA from the 5′ end, and in some cases does not inhibit polymerase activity, thus allowing the reaction to occur in one single process. The resulting single-stranded regions on adjacent DNA fragments can anneal. The DNA polymerase can incorporate nucleotides to fill in any gaps. The DNA ligase can covalently join the DNA of adjacent segments, thereby removing any nicks in the DNA. Either linear or closed circular molecules can be assembled. In some embodiments, PCR can be utilized to perform the Gibson assembly.

### Cloning of Recombinant Antigen-Binding Molecules

Existing antibody cloning methods can be time-consuming or difficult, and can require considerable automation and expensive plate-based reagents in order to succeed. Human labor can be used instead, but this can become impractical when cloning thousands of antibodies, which can a common procedure, for example during pandemic antibody discovery efforts and during antibody discovery campaigns for pharmaceutical research. Methods for enriching nucleic acid sequences (including those coding for antibodies or fragments thereof) and methods for cloning those sequences provided herein can leverage cDNA and amplified sequences to efficiently recover targets of interest from one or more single cell libraries. Using approaches described herein, users can sequence thousands to hundreds of thousands of antibodies and target select antibodies for recovery and cloning. This can be particularly powerful when combined with other components provided herein (e.g., barcoding) to screen, e.g. for antigen specificity or other multiomic data.

Methods provided herein can further comprise cloning a nucleic acid sequence of interest into a vector. A vector can be a nucleic acid (e.g., DNA) molecule used as a vehicle to artificially carry foreign genetic material into a cell, where it can be replicated and/or expressed. Examples of vectors can include a viral vector, a plasmid, a bacteriophage, a cosmid, or an artificial chromosome.

In some embodiments, a vector can be modified by the addition of genetic material coding for a protein. For example, a vector can comprise a nucleic acid sequence that can be combined with the nucleic acid sequence of interest. For example, a vector can comprise a nucleic acid sequence that can be combined with the nucleic acid sequence of interest to yield a nucleic acid sequence for a protein of interest, such as an antibody or antigen-binding fragment thereof, or B cell receptor. For example, a vector can comprise at least a portion of a constant region of a B cell receptor, or an antibody or antigen-binding fragment thereof.

In some embodiments, a vector can comprise a promoter. A promoter can be a sequence of DNA to which one or more proteins can bind that can initiate transcription of a single RNA from the DNA downstream of it. This RNA may encode a protein, or can have a function in and of itself, such as tRNA, mRNA, or rRNA. Promoters are located near the transcription start sites of genes, upstream on the DNA (towards the 5′ region of the sense strand). Promoters can be about 100-1000 base pairs long. Examples of promoters can include bacterial promoters or eukaryotic promoters.

In some embodiments, cloning can comprise a vector restriction digest (e.g., cutting of the nucleic acid sequence of the vector at a restriction site, or site recognized by a restriction enzyme). A restriction digest of a vector can comprise digesting the vector at a restriction site. A restriction site can be a DNA sequence on the vector that can contain a specific sequence of nucleotides (e.g., 4-8 bases long) that can be recognized by a restriction enzyme. In some embodiments, a restriction site can be a palindromic sequence. In some embodiments, a restriction enzyme (e.g., a restriction enzyme that can recognize the restriction site) can cut the sequence between two nucleic acids within the restriction site or nearby the restriction site. An example of a restriction site can be, for example, a FspI restriction site that can be recognized by the FspI restriction enzyme. Non-limiting examples of restriction sites that can be employed are provided in Table 2.

TABLE 2
Restriction enzymes and their recognition sequences

| | | Recognition | |
|---|---|---|---|
| Enzyme | Source | Sequence | Cut |
| EcoRI | Escherichia coli | 5′GAATTC | 5′---G AATTC---3′ |
| | | 3′CTTAAG | 3′---CTTAA G---5′ |
| EcoRII | Escherichia coli | 5′CCWGG | 5′---CCWGG---3′ |
| | | 3′GGWCC | 3′---GGWCC---5′ |
| BamHI | Bacillus amyloliquefaciens | 5′GGATCC | 5′---G GATCC---3′ |
| | | 3′CCTAGG | 3′---CCTAG G---5′ |
| HindIII | Haemophilus influenzae | 5′AAGCTT | 5′---A AGCTT---3′ |
| | | 3′TTCGAA | 3′---TTCGA A---5′ |
| TaqI | Thermus aquaticus | 5′TCGA | 5′---T CGA---3′ |
| | | 3′AGCT | 3′---AGC T---5′ |
| NotI | Nocardia otitidis | 5′GCGGCCGC | 5′---GC GGCCGC---3′ |
| | | 3′CGCCGGCG | 3′---CGCCGG CG---5′ |
| HinFI | Haemophilus influenzae | 5′GANTC | 5′---G ANTC---3′ |
| | | 3′CTNAG | 3′---CTNA G---5′ |
| Sau3 AI | Staphylococcus aureus | 5′GATC | 5′---GATC---3′ |
| | | 3′CTAG | 3′---CTAG---5′ |
| PvuII | Proteus vulgaris | 5′CAGCTG | 5′---CAG CTG---3′ |
| | | 3′GTCGAC | 3′---GTC GAC---5′ |
| SmaI | Serratia marcescens | 5′CCCGGG | 5′---CCC GGG---3′ |
| | | 3′GGGCCC | 3′---GGG CCC---5′ |
| HaeIII | Haemophilus aegyptius | 5′GGCC | 5′---GG CC---3′ |
| | | 3′CCGG | 3′---CC GG---5′ |
| HgaI | Haemophilus gallinarum | 5′GACGC | 5′---NN NN---3′ |
| | | 3′CTGCG | 3′---NN NN---5′ |
| AluI | Arthrobacter luteus | 5′AGCT | 5′---AG CT---3′ |
| | | 3′TCGA | 3′---TC GA---5′ |
| EcoRV | Escherichia coli | 5′GATATC | 5′---GAT ATC---3′ |
| | | 3′CTATAG | 3′---CTA TAG---5′ |
| EcoP15I | Escherichia coli | 5′CAGCAGN₂₅NN | 5′---CAGCAGN₂₅ NN--- |
| | | | 3′ |
| | | 3′GTCGTCN₂₅NN | 3′---GTCGTCN₂₅NN--- |
| | | | 5′ |
| KpnI | Klebsiella pneumoniae | 5′GGTACC | 5′---GGTAC C---3′ |
| | | 3′CCATGG | 3′---C CATGG---5′ |
| PstI | Providencia stuartii | 5′CTGCAG | 5′---CTGCA G---3′ |
| | | 3′GACGTC | 3′---G ACGTC---5′ |
| SacI | Streptomyces achromogenes | 5′GAGCTC | 5′---GAGCT C---3′ |
| | | 3′CTCGAG | 3′---C TCGAG---5′ |
| SalI | Streptomyces albus | 5′GTCGAC | 5′---G TCGAC---3′ |
| | | 3′CAGCTG | 3′-CAGCT G---5′ |
| ScaI | Streptomyces caespitosus | 5′AGTACT | 5′---AGT ACT---3′ |
| | | 3′TCATGA | 3′---TCA TGA---5′ |
| SpeI | Sphaerotilus natans | 5′ACTAGT | 5′---A CTAGT---3′ |
| | | 3′TGATCA | 3′---TGATC A---5′ |
| SphI | Streptomyces | 5′GCATGC | 5′---GCATG C---3′ |
| | phaeochromogenes | 3′CGTACG | 3′---C GTACG---5′ |
| StuI | Streptomyces tubercidicus | 5′AGGCCT | 5′---AGG CCT---3′ |
| | | 3′TCCGGA | 3′---TCC GGA---5′ |
| XbaI | Xanthomonas badrii | 5′TCTAGA | 5′---T CTAGA---3′ |
| | | 3′AGATCT | 3′---AGATC T---5′ |

A cloning vector can have features that can allow a gene to be suitably inserted into the vector or removed from it. Examples can include a multiple cloning site (MCS) or polylinker, which can contain unique restriction site(s). The restriction site(s) in the MCS can be first cleaved by restriction enzymes, then a PCR-amplified target gene also digested with the same enzymes is ligated into the vectors using DNA ligase. The target DNA sequence can be inserted into the vector in a specific direction if so desired. The restriction sites may be further used for sub-cloning into another vector if necessary.

Other cloning vectors may employ topoisomerase instead of ligase, and cloning can be performed more rapidly without the need for restriction digest of the vector or insert. In this TOPO cloning method, a linearized vector can be activated by attaching topoisomerase I to its ends, and this “TOPO-activated” vector may then accept a PCR product by ligating both the 5′ ends of the PCR product, releasing the topoisomerase and forming a circular vector in the process. Another method of cloning without the use of DNA digest and ligase can be by DNA recombination, for example as used in the Gateway cloning system. The gene, once cloned into the cloning vector, may be suitably introduced into a variety of expression vectors by recombination.

A vector can comprise a reporter gene. A reporter gene can be used in some cloning vectors to facilitate the screening of successful clones by using features of these genes that allow successful clone to be easily identified. Such features can include the lacZα fragment for a complementation in blue-white selection, and/or marker gene or reporter genes in frame with and flanking the MCS to facilitate the production of fusion proteins. Examples of fusion partners that may be used for screening can include the green fluorescent protein (GFP) and luciferase.

In some embodiments, cloning can comprise combining two or more nucleic acid sequences. For example, two or more nucleic acid sequences can be joined to yield an amino acid sequence of interest (e.g., a B cell receptor or an antibody or antigen-binding fragment thereof). Two or more nucleic acid sequences can comprise a nucleic acid sequence of a heavy chain of an antibody or antigen-binding fragment and a nucleic acid sequence of a light chain. In such a method, a full antibody or antigen-binding fragment thereof, B cell receptor, or other amino acid can be cloned in a single vector and expressed as a single nucleic acid sequence or amino acid sequence.

After cloning, the nucleic acid sequence of interest or the amino acid product of the nucleic acid sequence of interest can be expressed. Expression can be performed in any acceptable expression system, including a bacterial expression system, a yeast expression system, an insect cell expression system, a viral expression system, or a mammalian cell expression system. In some embodiments, expression can be in a live animal.

The protein product of the nucleic acid sequence of interest can be analyzed. For example, the affinity, specificity, enzymatic activity, solubility, stability, or other property of the protein product can be analyzed. Examples of assays can include ELISA, western blot, enzymatic assay, dot blot, Bradford protein assay, neutralization assay, immunoassay, or another assay.

In addition to generating desired antibodies via expression of nucleic acid molecules that have been altered by recombinant molecular biological techniques, a subject recombinant antigen-binding molecule (e.g., an antibody or a functional fragment thereof) in accordance with the present disclosure can be chemically synthesized. Chemically synthesized polypeptides are routinely generated by those of skill in the art.

Once assembled (by synthesis, recombinant methodologies, site-directed mutagenesis or other suitable techniques), the DNA sequences encoding a recombinant antigen-binding molecule (e.g., an antibody or a functional fragment thereof) as disclosed herein can be inserted into an expression vector and operably linked to an expression control sequence appropriate for expression of the recombinant antigen-binding molecule (e.g., an antibody or a functional fragment thereof) in the desired transformed host. Proper assembly can be confirmed by nucleotide sequencing, restriction mapping, and expression of a biologically active polypeptide in a suitable host. As is known in the art, in order to obtain high expression levels of a transfected gene in a host, care should be taken to ensure that the gene encoding the recombinant antigen-binding molecule (e.g., an antibody or a functional fragment thereof) is operably linked to transcriptional and translational expression control sequences that are functional in the chosen expression host.

In some embodiments, a method of the disclosure further involves including coupling a reporter oligonucleotide including a reporter barcode sequence to the recombinant antigen-binding molecule (e.g., an antibody or a functional fragment thereof) to generate a barcoded recombinant antigen-binding molecule (e.g., an antibody or a functional fragment thereof). In some embodiments, the reporter barcode sequence of the reporter oligonucleotide includes a unique identifier for the recombinant antigen-binding molecule (e.g., an antibody or a functional fragment thereof). In some embodiments, the unique identifier for the recombinant antigen-binding molecule is a nucleic acid identifier. In some embodiments, the method of the disclosure further including determining all or a part of the nucleic acid identifier to identify the barcoded recombinant antigen-binding molecule (e.g., an antibody or a functional fragment thereof). In some embodiments, the reporter oligonucleotide comprise an adapter region that allows for downstream analysis of the recombinant antigen-binding molecule (e.g., an antibody or a functional fragment thereof). In some embodiments, the adapter region comprises a primer binding site and/or a cleavage site.

### C. Methods of Treatment

In some embodiments, the methods of the disclosure further include administering a therapeutic composition including a recombinant antigen-binding molecule (e.g., an antibody or a functional fragment thereof) as described herein and/or an engineered cell (e.g., an engineered immune cell) expressing the recombinant antigen-binding molecule as described herein to a subject in need thereof. In some embodiments, the therapeutic composition is formulated to be compatible with its intended route of administration. For example, the recombinant antigen-binding molecules of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject recombinant antigen-binding molecules of the disclosure can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

For example, the data obtained from the cell culture assays and animal studies can be used in formulating a range of dosage for use in humans. The dosage of such compounds lies generally within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

D. Methods of Engineering an Antibody, or Antigen-Binding Fragment Thereof to have an Altered Characteristic/Methods of Preparing a Library of Variant Antibodies, or Variant Antigen-Binding Fragments Thereof; Having an Altered Characteristic

### Providing a Nucleic Acid Sequence

The present disclosure further provides methods of engineering an antigen-binding site of an antibody, or antigen-binding fragment thereof, to comprise an altered characteristic. Additionally, the present disclosure provides methods of preparing a library of variant antibodies, or variant antigen-binding fragments thereof, comprising an altered characteristic or characteristics.

In these methods, a nucleic acid sequence encoding a selected antibody, or selected antigen-binding fragment thereof is provided. The selected antibody, or selected antigen-binding fragment thereof, encoded by the provided nucleic acid sequence may be a “full antibody”, as is typically expressed by most mammals including humans, e.g., an immunoglobulin (Ig) molecule including four polypeptide chains, two heavy chains (HCs) and two light chains (LCs) inter-connected by disulfide bonds, or a multimer thereof (e.g. IgM). The selected antibody, as described earlier herein, may be an IgA (e.g., IgA1 or IgA2), IgD, IgE, IgG (e.g., IgG1, IgG2, IgG3 and IgG4) or IgM antibody.

The selected antibody, or selected antigen-binding fragment thereof, (e.g., antigen-binding fragment of the selected antibody) encoded by the provided nucleic acid sequence, may be the antigen-binding fragment of the selected antibody. If it is the antigen-binding fragment of the selected antibody, then it may be any naturally occurring, enzymatically obtainable, synthetic, or genetically engineered polypeptide or glycoprotein derivative of the selected antibody. An antigen-binding fragment of the selected antibody, as may be the case in other of the embodiments of the compositions and methods herein, may be any of a: (i) Fab fragment; (ii) F(ab′)2 fragment; (iii) Fd fragment; (iv) Fv fragment; (v) single-chain Fv (scFv) molecule; (vi) sdAb fragment; or (vii) minimal recognition unit consisting of the amino acid residues that mimic the hypervariable region of the selected antibody (e.g., an isolated complementarity determining region (CDR) such as a CDR3 peptide), or a constrained FWR3-CDR3-FWR4 peptide. Further, the antigen-binding fragment of the selected antibody may be an engineered molecule, such as a domain-specific antibody, single domain antibody, chimeric antibody, CDR-grafted antibody, diabody, triabody, tetrabody, minibody, nanobody (e.g., monovalent nanobodies, bivalent nanobodies, etc.), a small modular immunopharmaceutical (SMIP), or a shark immunoglobulin new antigen receptor (IgNAR) variable domain.

Further, if the selected antibody, or selected antigen-binding fragment thereof, encoded by the provided nucleic acid sequence is an antigen-binding fragment of the selected antibody, the antigen-binding fragment of the selected antibody, as may be the case in other of the embodiments of the compositions and methods herein, may be a monomeric V_{H} or V_{L} domain of the selected antibody, or a configuration of a variable domain of the selected antibody with one or more constant domains, including any of a: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, the variable and constant domains can be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Furthermore, any of the selected antibodies or selected antigen-binding fragments thereof can be mono-specific or multi-specific (e.g., bi-specific).

In some embodiments of methods providing the nucleic acid sequence of a selected antibody, (or selected antigen-binding fragment thereof), the selected antibody, (or selected antigen-binding fragment thereof), is a human antibody. In some embodiments, the selected antibody, or selected antigen-binding fragment thereof, is an antigen-binding fragment of a human antibody. In other embodiments, the selected antibody, or selected antigen-binding fragment thereof, is a humanized antibody. In yet other embodiments, the selected antibody, or selected antigen-binding fragment thereof, is an antigen-binding fragment of a humanized antibody. In further still embodiments, the selected antibody, or selected antigen-binding fragment thereof, is a mouse antibody, or is an antigen-binding fragment of a mouse antibody. In other embodiments, the selected antibody, or selected antigen binding fragment thereof may be an antibody or antigen-binding fragment of a rabbit, goat, rat, sheep, horse, cow, llama, alpaca, camel, chicken or shark antibody.

The selected antibody, or selected antigen-binding fragment thereof, may be selected due to its ability to bind a target antigen. The target antigen to which the selected antibody, or antigen-binding fragment thereof, may bind may be any antigen for which the development and identification of variants of the selected antibody, or selected antigen-binding fragment thereof, is desirable. The target antigen, as may be the case in other of the embodiments of the compositions and methods herein, may be an antigen associated with a pathogen or infectious agent, such as a viral, bacterial, parasitic, protozoal or prion agent. If the target antigen is associated with an infectious agent that is a viral agent, the viral agent may be an influenza virus, a coronavirus, a retrovirus, a rhinovirus, or a sarcoma virus. The viral agent may be severe acute respiratory syndrome coronavirus 1 (SARS-CoV-1), a SARS-CoV-2, a Middle East respiratory syndrome coronavirus (MERS-CoV)), or human immunodeficiency virus (HIV), influenza, respiratory syncytial virus, or Ebola virus. If the target antigen is associated with an infectious agent that is a viral agent, the target antigen may be corona virus spike (S) protein, an influenza hemagglutinin protein, an HIV envelope protein or any other a viral glycoprotein. Further, the target antigen may be associated with a tumor or a cancer. If the target agent is associated with a tumor or cancer, it may be, for example, a growth factor or a growth factor receptor. Examples of target antigens that may be associated with tumors or cancers include epidermal growth factor receptor (EGFR), CD38, platelet-derived growth factor receptor (PDGFR) alpha, insulin growth factor receptor (IGFR), CD20, CD19, CD47, or human epidermal growth factor receptor 2 (HER2). Alternatively, the target antigen may be an immune checkpoint molecule that may or may not be associated with tumors or cancers (e.g., CD38, PD-1, CTLA-4, TIGIT, LAG-3, VISTA, TIM-3), or it may be a cytokine, a GPCR, a cell-based co-stimulatory molecule, a cell-based co-inhibitory molecule or an ion channel. Other examples of a target antigen include autoantigens, virus-like particles and lipoparticles. Further still, the target antigen may be associated with a degenerative condition or disease (e.g., an amyloid protein or a tau protein).

The selected antibody, or selected antigen-binding fragment thereof, may be selected due to its binding a fragment of the target antigen that includes a region of interest, e.g., target antigen region of interest. The target antigen region of interest may be a particular domain, domains, epitope or epitopes of the target antigen. The target antigen region of interest may be a fragment of the target antigen that is a 10-200, 20-200, a 20-180, a 20-160, a 20-140, a 20-120, a 20-100, a 20-80, a 20-60, a 20-40, a 40-200, a 40-180, a 40-160, a 40-140, a 40-120, a 40-100, a 40-80, a 40-60, 60-200, a 60-180, a 60-160, a 60-140, a 60-120, a 60-100, a 60-80, a 80-200, a 80-180, a 80-160, a 80-140, a 80-120, a 80-100, a 100-200, a 150-100, or a 25-175 amino acid residue peptide region of the target antigen. The fragment of the target antigen may have an amino acid length that is 95%, 90%, 85%, 80%, 75%, 70%, 65%, 60%, 55%, 50%, 45%, 40%, 35%, 30%, 25%, 20%, 15%, 10%, or 5% that of the target antigen. The region of interest of the target antigen may include or may be an epitope of the target antigen, e.g., a linear or conformational or cryptic epitope. The region of interest of the target antigen may include or may be a domain of the target antigen, e.g., a unit or portion the antigen that is self-stabilizing and folds independently of the remainder of the antigen (and which can be determined by, for example, Hydrophobicity/Kyte-Doolittle plots, InterPro or PROSITE (www.ebi.ac.uk/interpro/) or protein BLAST).

The selected antibody, or selected antigen-binding fragment thereof, may be selected due to its binding the target antigen at a region of interest that includes one or more epitopes or domains of the target antigen that are involved in a signaling pathway, that interact with other proteins or peptides, or that result in or prevent a conformational change in the target antigen.

The provided nucleic acid sequence encoding the selected antibody, or selected antigen-binding fragment thereof, that binds the target antigen as described herein may be provided as a nucleic acid sequence of any type, e.g., DNA/cDNA molecule, suitable for amplification. The provided nucleic acid sequence encoding the selected antibody, or selected antigen-binding fragment thereof, may be provided as a plurality, or library, of nucleic acid sequences, e.g., DNA/cDNA molecules. The provided nucleic acid sequence encoding the selected antibody, or selected antigen-binding fragment thereof, may be provided as a nucleic acid sequence, e.g., DNA/cDNA, molecule cloned into a vector. The provided nucleic acid sequence encoding the selected antibody, or selected antigen-binding fragment thereof, may be provided as a plurality of nucleic acid sequences, e.g., DNA/cDNA molecules, cloned into a plurality of vectors. The provided nucleic acid sequence encoding the selected antibody, or selected antigen-binding fragment thereof, may be configured in such a way as to facilitate amplification by any method, e.g., including rolling circle amplification.

The provided nucleic acid sequence encoding the selected antibody, or selected antigen-binding fragment thereof, may have been derived, (or, alternatively, may have been obtained, discovered or sequenced), from a cell of a vertebrate. For example, the provided nucleic acid sequence encoding the selected antibody, or selected antigen-binding fragment thereof, may be derived from a cell or a sample of cells of a mammal, reptile, or fish. If the selected antibody, or selected antigen-binding fragment thereof, is derived from a cell of a mammal, it may be derived from a cell or a sample or cells of a human, a mouse, a rabbit, a goat, a rat, a sheep, a horse, a cow, a llama, an alpaca or a camel. If the selected antibody, or selected antigen-binding fragment thereof, is derived from a cell, or a sample of cells of the mouse, the rat, the rabbit, the chicken or the cow, then the mouse, rat, rabbit, chicken or cow may be transgenic and may be transgenic to express human antibodies. If the selected antibody, or selected antigen-binding fragment thereof, is derived from a cell of a fish, it may be derived from a cell or a sample of cells of a jawed fish, such as a shark. The cell or the sample of cells of the vertebrate from which the nucleic acid sequence encoding the selected antibody, or selected antigen-binding fragment thereof, may be derived may have been a cell of the B cell lineage, or of a sample of cells that includes cells of B cell lineage, e.g., memory B cells.

The cell, or sample of cells, from which the provided nucleic acid sequence encoding the selected antibody, or selected antigen-binding fragment thereof is derived may be a cell obtained from a donor, or a sample of cells obtained from a donor, such as a human donor. A cell or a sample of cells from which the provided nucleic acid sequence encoding the selected antibody, or selected antigen-binding fragment thereof, is derived may be a cell or a sample of cells obtained from a mouse, a transgenic mouse, a rat, a transgenic rat, a rabbit, a transgenic rabbit, a goat, a sheep, a horse, a cow, a transgenic cow, a llama, an alpaca, a camel, a chicken, a transgenic chicken, or a shark. If the cell is of a sample of cells, the sample may be a blood sample, a peripheral blood mononuclear cell sample or a plasma sample. By way of example, the sample of cells may be a blood sample of a human donor or a mouse, e.g., a transgenic mouse. The sample of cells may be a peripheral blood mononuclear cell sample of a human donor or a mouse, e.g., a transgenic mouse. The sample of cells may be a plasma sample of a human or a mouse, e.g., a transgenic mouse.

The cells, or the sample of cells, from which the provided nucleic acid sequence encoding the selected antibody, or selected antigen-binding fragment thereof, is derived need not be derived from a cell or a cell sample of a single donor. The cells or the sample of cells may be obtained by combining cells, or samples of cells, of multiple donors, e.g., cells or cell samples of more than one human, cells or cell samples of more than one mouse, cells or cell samples of more than one rat, cells or cell samples of more than one rabbit, cells or cell samples of more than one goat, cells or cell samples of more than one sheep, cells or cell samples of more than one horse, cells or cell samples of more than one cow, cells or cell samples of more than one llama, cells or cell samples of more than one alpaca, cells or cell samples of more than one camel or cells or cell samples of more than one shark. If the cells or the samples of cells are of multiple donors, the cells or samples of cells may be any combination of one or more of blood samples, peripheral blood mononuclear cell samples or plasma samples. If the cells or the samples of cells are of multiple donors, the cells or samples of cells may be any combination of one or more of human blood samples, human peripheral blood mononuclear cell samples or human plasma samples. If the cells or the samples of cells are of multiple donors, the cells or samples of cells may be any combination of one or more of mouse blood samples, mouse peripheral blood mononuclear cell samples or mouse plasma samples. If the cells or the samples of cells are of multiple donors, the cells or samples of cells may be any combination of one or more of transgenic mouse blood samples, transgenic mouse peripheral blood mononuclear cell samples or transgenic mouse plasma samples.

The donor, from which the cells or the sample of cells is obtained, may be known to have been exposed to the target antigen. Alternatively, the donor, from which the cells or the sample of cells is obtained, may be a donor suspected of having been exposed to the target antigen. The donor, from which the cells or the sample of cells is obtained, may be known or expected to be resistant to a pathogen or infectious agent that bears the target antigen.

It is to be understood that the nucleic acid sequence encoding the selected antibody, or selected antigen-binding site thereof, need not have been derived from a donor, a cell obtained from a donor or a cell sample of a donor. The nucleic acid sequence encoding the selected antibody, or selected antigen-binding site thereof, may have been derived from any source including a sequence of an antibody purchased from a vendor, a therapeutic antibody known in the art (e.g., antibodies disclosed in Table 1), a sequence having been selected from a phage or yeast scFv library, or a sequence synthesized based on a published disclosure.

### Error-Prone Amplification

In the methods in which a nucleic acid sequence or sequences encoding the selected antibody, or selected antigen-binding fragment thereof are provided, the sequences may be amplified in an error-prone amplification reaction. The error-prone amplification reaction may be conducted to introduce errors in the sequence encoding the selected antibody, or antigen-binding fragment thereof, resulting in one or more amino acid alterations, e.g., substitutions, insertions or deletions in the amino acid sequence of the selected antibody, or selected antigen-binding fragment thereof. The error-prone amplification reaction may also, or alternatively, result in an alteration that truncates the amino acid sequence of the selected antibody, or selected antigen-binding fragment thereof. The error-prone amplification may introduce errors resulting one, at least one, two, at least two, three, at least three, four, at least four, five, at least five, six, at least six, seven, at least seven, eight, at least eight, nine, at least nine, ten, at least ten, at most five, at most ten, at most fifteen, or at most twenty amino acid residue alterations in the selected antibody, or antigen-binding fragment thereof. The error-prone amplification may introduce errors resulting one to twenty, one to fifteen, one to ten, one to five, five to ten, five to fifteen, five to twenty, or five to fifteen amino acid residue alterations in the selected antibody, or antigen-binding fragment thereof. The error-prone amplification may introduce errors at a rate of 1-50 mutations/kilobase, 1-40 mutations/kilobase, 1-30 mutations/kilobase, 1-20, 10-50 mutations/kilobase mutations/kilobase, 20-50 mutations/kilobase, 30-50 mutations/kilobase or 20-40 mutations/kilobase in the provided nucleic acid sequence encoding the selected antibody, or selected antigen-binding fragment thereof. The introduction of the amino acid alterations in the selected antibody, or selected antigen-binding fragment thereof produces variant antibodies or variant antigen-binding fragments thereof, e.g., a plurality of variant antibodies or variant antigen-binding fragments thereof.

Error-prone amplification can be performed under any set of conditions well known in the art to increase the likelihood of introduction of errors during an amplification reaction. Conditions that increase the likelihood of introduction of errors during an amplification reaction, e.g., conditions conducive to error-prone amplification, include decreased quantity of template nucleic acid, imbalanced dNTP ratios, increased MgCl₂ concentration, inclusion of MnCl₂ and utilization of a polymerase without (or with diminished) proofreading capabilities, during the amplification reaction. If the likelihood of introduction of errors during an amplification reaction is to be increased is via decreased quantity of the template nucleic acid, the template nucleic acid may be present in the reaction at a total quantity of 500-1000, 550-1000, 600-1000, 650-1000, 700-1000, 750-1000, 800-1000, 850-1000, 900-1000, 950-1000, 500-550, 550-600, 600-650, 650-700, 700-750, 750-800, 800-850, 850-900, 900-950 nanograms. If the likelihood of introduction of errors during an amplification reaction is to be increased via decreased quantity of the template nucleic acid, the template nucleic acid present in the reaction may be a total quantity of 100-500, 150-500, 200-500, 250-500, 300-500, 350-500, 400-500, 450-500, 100-150, 150-200, 200-250, 250-300, 300-350, 350-400, 400-450, 450-500 nanograms. Alternatively, the likelihood of introduction of errors during an amplification reaction is to be increased is via decreased quantity of the template nucleic acid, the template nucleic acid present in the reaction may be a total quantity of 0.1 to 100, 1-100, 10-100, 20-100, 30-100, 40-100, 50-100, 60-100, 70-100, 80-100, 90-100, 0.1-10, 0.1-20, 0.1-30, 0.1-40, 0.1-50, 0.1-60, 0.1-70, 0.1-80, 0.1-90, 0.1-0.5, 0.1-0.4, 0.1-0.3, 0.1-0.2 nanograms.

If the likelihood of introduction of errors during an amplification reaction is to be increased is via “unbalanced dNTP concentration” in the amplification reaction, the “unbalanced dNTP concentration” may be one in which more dCTP and dTTP than dATP and dGTP is present in the amplification reaction. For example, dATP and dGTP may be included at 0.2-5, 1-5, 2-5-, 3-5, 4-5, 0.2-0.3, 0.3-0.4, 0.4-0.5, 0.5-0.6, 0.6-0.7, 0.7-0.8, 0.8-1 mM in the reaction, while dCTP and dTTP may be included at 0.2-25, 1-25, 5-25, 10-25, 15-25, 20-25, 0.2-0.3, 0.3-0.4, 0.4-0.5, 0.5-0.6, 0.6-0.7, 0.7-0.8, 0.8-1 mM in the reaction.

If the error-prone amplification reaction is performed to increase the likelihood of introduction of errors via increasing MgCl₂ concentration, the MgCl₂ concentration may be increased to 1-25, 1-20, 1-15, 1-10, 1-5, 1, 2, 3, 4, 5, 6, 7, 8, 9, 10, 11, 12, 13, 14, 15, 16, 17, 18, 19, 20, 21, 22, 23, 24, 25 mM. If the error-prone amplification reaction is performed to increase the likelihood of introduction of errors via including MnCl₂ in the reaction, MnCl₂ may be included in the reaction at a concentration of 10-200, 20-200, 30-200, 40-200, 50-200, 60-200, 70-200, 80-200, 90-200, 100-200, 125-200, 125-200, 150-200, 175-200, 10-20, 10-30, 10-40, 10-50, 10-60, 10-70, 10-80, 10-90, 10-100, 10-110, 10-120, 10-130, 10-140, 10-150, 10-160, 10-170, 10-180, 10-190 mM.

If the likelihood of introduction of errors during an amplification reaction is to be increased due to the inclusion of an error-prone polymerase, the error-prone polymerase may be a polymerase lacking, or with diminished, proof-reading capability. Examples of such polymerases include Mutazyme™ I and Mutazyme™ II (Agilent) or Taq DNA polymerase (NEB).

Kits for performing error-prone amplification are also commercially available to those of skill in the art and include PickMutant™ Error Prone PCR Kit (Canvax), JBS Error-Prone Kit (Jena Bioscience GmBH), GeneMorph II Random Mutagenesis Kit (Agilent).

In an embodiment, the error-prone amplification may be performed by rolling circle amplification. The rolling circle amplification may be conducted in a reaction mixture comprising a DNA polymerase, template DNA, random hexamers (for reaction priming), dNTPs, and a buffer containing MnCl₂, wherein the MnCl₂ concentration is responsible for increasing likelihood of introducing errors. The MnCl₂ may be included in the reaction at 1-15, 1-10, 1-5, 1, 2, 3, 5, 6, 7, 8, 9, 10, 11, 12, 13, 14, 15 mM, with higher concentrations increasing likelihood of introducing errors. Additional details of error-prone amplification by rolling circle amplification can be found in Fujii et al., Nature Protocols 5(2006):2493-2497, incorporated herein by reference.

### Expressing the Plurality of Variant Antibodies or Variant Antigen Binding Fragments Thereof

In any of the methods provided herein, the error-prone amplification produces a plurality of polynucleotides encoding variant antibodies, or variant antigen-binding fragments thereof. The plurality of variant antibodies, or variant antigen-binding fragments thereof, may be expressed in a plurality of engineered cells. The expression may be such that an engineered cell of the plurality of engineered cells expresses a variant antibody, or variant antigen-binding fragment thereof, of the plurality of variant antibodies, or variant antigen-binding fragments thereof. The plurality of engineered cells may comprise a nucleic acid construct one or more of one or more of (a) a first heterologous nucleic acid sequence encoding a stgRNA operably linked to a first promoter, (b) a second heterologous nucleic acid sequence encoding a CRISPR-associated (Cas) protein operably linked to a second promoter, wherein the second promoter is induced by an immune response; and (c) a third nucleic acid sequence encoding an antigen-binding molecule of interest, wherein the antigen-binding molecule of interest is the variant antibody, or variant antigen-binding fragment thereof. The nucleic acid constructs, further characteristics of the elements ((a), (b) and (c)) of the nucleic acid constructs, and additional elements that may be included in the constructs have been described earlier herein.

The expression of the variant antibodies, or variant antigen-binding fragments thereof, may be in any suitable engineered cell for antibody expression and in which an immune response may be induced, many of which have been described herein. The incorporation of nucleic acid sequences, including those of antigen-binding molecules, (e.g., such as variant antibodies, or variant antigen-binding fragments thereof), into expression cassettes and vectors have been described elsewhere in the instant disclosure. Similarly, introduction of the expression cassettes and/or vectors into cells have already been described in the instant disclosure.

Contacting the Engineered Cell with the Target Antigen/Determining Mutations/Identifying

In embodiments of the methods provided herein, the plurality of engineered cells expressing the variant antibodies, or variant antigen-binding fragments thereof, may be incubated in a reaction mixture with the target antigen. In such embodiments, the binding of the target antigen with the variant antibody, or variant antigen-binding fragment thereof, may activate an immune response that induces expression the Cas protein. The induced Cas protein introduces one or more mutations in the nucleic acid sequence of the stgRNA. The number of mutations introduced in the nucleic acid sequence of the stgRNA are determined.

The number of mutations introduced by inducing expression of the Cas protein may be determined by any method known in the art, and as described earlier herein, e.g., including sequencing all or a part of the nucleic acid sequence encoding the stgRNA, which may or may not occur as part of, or in the context of, whole transcriptome sequencing. The number of mutations introduced in the sequence of the all or a part of the nucleic acid sequence encoding the stgRNA may be determined to be at least 1 mutation, at least 2 mutations, at least 3 mutations, at least 4 mutations, at least 5 mutations, at least 6 mutations, at least 7 mutations at least 8 mutations, at least 9 mutations, or at least 10 mutations. The number of mutations introduced in the sequence of the all or a part of the nucleic acid sequence encoding the stgRNA may be determined to be between 1 and 10 mutations, or 2 and 10 mutations, or 3 and 10 mutations, or 4 and 10 mutations, or 5 and 10 mutations, or 6 and 10 mutations, or 7 and 10 mutations, or 8 and 10 mutations, or 2 and 9 amutations, or 4 and 8 mutations, or 3 and 7 mutations.

If the number of mutations introduced in all or a part of the sequence encoding the stgRNA identifies the variant antibody, or variant antigen-binding fragment thereof, as engineered to include the altered characteristic (or identifies the variant antibody, or variant antigen binding fragment thereof, for inclusion in the library), the number of mutations introduced that identifies the variant antibody, or variant antigen-binding fragment thereof may be at least 1 mutation, at least 2 mutations, at least 3 mutations, at least 4 mutations, at least 5 mutations, at least 6 mutations, at least 7 mutations at least 8 mutations, at least 9 mutations, or at least 10 mutations. Alternatively, the number of mutations introduced that identifies the variant antibody, or variant antigen-binding fragment thereof, as engineered to include the altered characteristic (or that identify the variant antibody, or variant antigen binding fragment thereof, for inclusion in the library) may be determined to be between 1 and 10 mutations, or 2 and 10 mutations, or 3 and 10 mutations, or 4 and 10 mutations, or 5 and 10 mutations, or 6 and 10 mutations, or 7 and 10 mutations, or 8 and 10 mutations, or 2 and 9 amutations, or 4 and 8 mutations, or 3 and 7 mutations. In this instance, the altered characteristic identified for the variant antibody, or variant antigen-binding fragment thereof, (or that identified the variant antibody, or variant antigen-binding fragment thereof, for inclusion in the library), may be increased affinity for the target antigen.

To identify the variant antibody, or variant antigen binding fragment thereof, as including the altered characteristic (or for inclusion in the library), the number of mutations introduced in all or a part of the sequence encoding the stgRNA in an engineered cell expressing the variant antibody, or variant antigen-binding fragment thereof, may be compared to number of mutations introduced in all or a part of the sequence encoding the stgRNA in a reference engineered cell. The reference engineered cell may be an engineered cell that (a) expresses the selected antibody, or selected antigen-binding fragment thereof as the antigen-binding molecule; (b) expresses an antigen-binding molecule that does not bind to the target antigen; (c) does not express an antigen-binding molecule; or (d) is an engineered cell whose the coding sequence for Cas protein is not switched on.

If the number of mutations introduced in all or a part of the sequence encoding the stgRNA in an engineered cell expressing a variant antibody, or variant antigen-binding fragment thereof, is compared to that of a reference engineered cell (e.g., engineered cell that expresses the selected antibody, or selected antigen-binding fragment thereof), then the variant antibody, or variant antigen-binding fragment thereof may be identified as having the altered characteristic (or identified for inclusion in the library) if it has at least as many as 2×, at least 2.5×, at least 3×, at least 3.5×, at least 4×, at least 5×, at least 5.5×, at least 6×, at least 6.5×, at least 7×, at least 7.5×, at least 8×, at least 8.5×, at least 9×, at least 9.5×, or at least 10× more mutations in the all or a part of the sequence encoding the stgRNA than does the reference engineered cell. In this instance, the altered characteristic identified for the variant antibody, or variant antigen-binding fragment thereof, may be increased affinity for the target antigen. If the altered characteristic is affinity, and the affinity is increased affinity, the increase in affinity may be a 10%, 20%, 30%, 40%, 50%, 60%, 70%, 80%, 90%, 100%, 10-fold, 20-fold, 30-fold, 40-fold, 50-fold, 60-fold, 70-fold, 80-fold, 90-fold, 100-fold or 1000-fold increase over that of the selected antibody, or selected antigen-binding fragment thereof.

If the number of mutations introduced in all or a part of the sequence encoding the stgRNA in an engineered cell expressing a variant antibody, or variant antigen-binding fragment thereof, is compared to that of a reference engineered cell (e.g., engineered cell that expresses the selected antibody, or selected antigen-binding fragment thereof), then the variant antibody, or variant antigen-binding fragment thereof may be identified as having the altered characteristic (or identified for inclusion in the library) if it has the same number as, or at most 1, at most 2, at most 3, at most 4 or at most 5 fewer mutations introduced in all or a part of the sequence encoding the stgRNA as does the reference engineered cell. In this instance, the altered characteristic identified for the variant antibody, or variant antigen-binding fragment thereof, may or may not (due to assay variability) be decreased affinity for the target antigen. A variant antibody (or variant antigen-fragment thereof) identified as a having an altered characteristic of decreased (or potentially decreased) affinity for the target antigen may be acceptable if the affinity of the variant antibody, or variant antigen-binding fragment thereof, for the target antigen remains in a tolerable range and the identified variant antibody, or variant antigen-binding fragment thereof, exhibits further altered, characteristics such as an altered association constant, an altered dissociation constant, or altered specificity for the target antigen, (such as a specificity alteration from multispecificity to more selective binding to the target antigen relative to the selected antibody, or selected antigen-binding fragment thereof). In such an instance, the affinity be decreased affinity, the decrease in affinity may be an at most 50%, 40% 30%, 20%, 15%, 10%, 9%, 8%, 7%, 6%, 5%, 4%, 3%, 2%, or 1% decrease in affinity.

A variant antibody (or variant antigen-fragment thereof) identified as a having an altered characteristic of decreased (or potentially decreased) affinity for the target antigen may be acceptable if the affinity of the variant antibody, or variant antigen-binding fragment thereof, for the target antigen is reduced by 100%. In such an instance it is desirable for the variant antibody, or variant antigen-binding fragment thereof, if multispecific, to exhibit selective binding to a second, but not the, target antigen.

If the identified altered characteristic of the variant antibody, or variant antigen-binding fragment thereof, is affinity that is determined by number of mutations introduced in all or a part of the sequence encoding the stgRNA, then binding affinity may be confirmed by other techniques that determine affinity of antigen-binding molecules for target proteins and/or their regions of interest including, for example, competition binning and competition enzyme-linked immunosorbent assay (ELISA), NMR or HDX-MS, Surface Plasmon Resonance (SPR), e.g. by using a Biacore™ system, or KinExA.

It will be understood that an altered characteristic of a variant antibody, or variant antigen-binding fragment thereof, may be, or may further include an alteration in an activity mediated by the variant relative to the selected antibody, or antigen-binding fragment thereof. An altered activity may be altered neutralization, if the selected antibody, or selected antigen-binding fragment thereof, is selected due to its binding to a target antigen that is associated with a pathogen, e.g., a virus. An altered activity may be altered autoimmune activity, if the selected antibody, or selected antigen-binding fragment thereof, is selected due to its binding to a target antigen that is associated with inflammation, e.g., cytokine or cytokine receptor. An altered activity may be altered anti-tumor activity, if the selected antibody or selected antigen-binding fragment thereof, is selected due to its binding a target antigen that is associated with a growth factor or growth factor receptor implicated in cancer, e.g., EGFR or IGFR. Activity assays suitable for determining these activities are known in the art.

### Partitioning

In certain embodiments of these methods, as in other methods provided herein, cells may be partitioned. In these certain embodiments of the methods provided herein, an engineered cell of the plurality of engineered cells expressing a variant antibody (or variant antigen-binding fragment thereof) of the plurality of variant antibodies (or variant antigen-binding fragments thereof) may be partitioned into a partition of a plurality of partitions. Further, if the methods further employ a reference engineered cell, the reference engineered cell may be partitioned in a partition of the plurality of partitions. The partitioning may occur prior to, or after, the engineered cells expressing the variant antibody, or variant antigen-binding fragment thereof (and/or, if included, the reference engineered cell(s)) are contacted with the target antigen. The partitioning may also be referred to as the compartmentalization or depositing of the cells (or nuclei of the cells) into discrete compartments or partitions, where each partition maintains separation of its own contents from the contents of other partitions. Examples of partitions include a droplet or well.

The partitions may further include a plurality of nucleic acid barcode molecules comprising a partition-specific barcode sequence, and that may further include a capture sequence and/or a functional sequence (e.g., useful for priming a sequencing reaction). In the methods including the partitioning, and in which the partitions (as a result of the partitioning) include the plurality of nucleic acid barcode molecules, barcoded nucleic acid molecules may be generated. The barcoded nucleic acid molecules may include a nucleic acid sequence of an analyte of the engineered cell or nucleus thereof, or reverse complement thereof and the partition-specific barcode sequence or reverse complement thereof. The analyte may be an mRNA or a DNA analyte. The analyte may be derived from any one or more of the first, the second or the third nucleic acid sequences of the nucleic acid construct. If the analyte is an mRNA or a DNA analyte, the capture sequence may couple to the mRNA or DNA analyte by complementary base pairing. The capture sequence may further include a template switch oligonucleotide sequence.

If the analyte is the mRNA analyte, then the capture sequence configured to couple to the mRNA analyte may include a polydT sequence. If the analyte is a cDNA analyte reversed transcribed from a cell mRNA (e.g., mRNA from a cell nucleus), then the capture sequence may be configured to couple to non-templated nucleotides appended to the cDNA during reverse transcription of the mRNA. If the cDNA analyte is reversed transcribed from the cell mRNA, then the cDNA analyte may have been reversed transcribed utilizing a primer comprising a polydT sequence. If the cDNA analyte is reversed transcribed from the cell mRNA, the reverse transcribing may result in appending of a non-templated cytosine, or two cytosines, or three cytosines, or at least three cytosines to the cDNA. If a non-templated cytosine, or two cytosines, or three cytosines, or at least three cytosines is appended to the cDNA during reverse transcription, the capture sequence configured to couple to the cDNA may be a guanine. Coupling of the capture sequence, e.g., including the guanine, to the non-templated cytosine permits reverse transcription of the cDNA to extend into the nucleic acid barcode molecule.

The barcoded nucleic acid molecules generated in the partition may be subject to subsequent reactions examples of such reactions have been described, in detail, throughout the disclosure herein. By way of example, the subsequent reactions may include sequencing reactions as have been described. The sequencing reactions may sequence all or a part of the nucleic acid sequence encoding the stgRNA. The sequencing reactions may also, or alternatively, sequence the nucleic acids encoding the variant antibody, or variant antigen-binding fragment thereof, e.g., the antigen-binding molecule of interest as the third nucleic acid sequence of the nucleic acid construct. If the sequencing reaction sequences the nucleic acid encoding the variant antibody, or variant antigen-binding fragment thereof, the sequencing reaction may determine a nucleic acid sequence of a complementarity determining region (CDR), a framework (FWR), a variable heavy chain domain (VH), and/or a variable light chain domain (VL) of the variant antibody or variant antigen-binding fragment thereof.

Here, as in other methods that may include a step of partitioning, it will be understood that the partitioning of biological particles (e.g., cells, cell beads, or cell nuclei) may partition more than one cell of the plurality of biological particles into more than one of a plurality of partitions. The partitioning may partition a first biological particle of the plurality of cells into a first partition, it may further partition a second biological particle of the plurality of biological particles into a second partition. Moreover, it may additionally partition a third biological particle of the plurality of biological particles into a third partition, a fourth biological particle of the plurality of biological particles into a fourth partition, up to hundreds, thousands, tens of thousands, or more biological particles that are partitioned into separate, individual, partitions. It should be understood that, in the partitioning, it is possible that not all partitions will include a biological particle. It should also be understood that, in the partitioning, not all partitions that include a biological particle (e.g., cell) will have the cell bound to the target antigen. Nonetheless, in case wherein the biological particles are cells, at least one cell of the population of cells partitioned into a partition will be bound to the target antigen.

A more detailed understanding of steps of methods provided herein that include embodiments and elements related to partitions, partitioning, reporter oligonucleotides and generation of barcoded nucleic acid molecules are provided in the sections that immediately follow.

### D. Systems and Methods for Partitioning

In an aspect, the systems and methods described herein provide for the compartmentalization, depositing, or partitioning of one or more particles (e.g., biological particles, such as cells, cell beads, or nuclei, macromolecular constituents of biological particles, beads, reagents, etc.) into discrete compartments or partitions (referred to interchangeably herein as partitions), where each partition maintains separation of its own contents from the contents of other partitions.

In some embodiments disclosed herein, the partitioned particle is a labelled cell of B-cell lineage (e.g., a plasma cell or memory B cell), which expresses an antigen-binding molecule (e.g., an immune receptor, an antibody or a functional fragment thereof). In other examples, the partitioned biological particle can be a labelled cell engineered to express antigen-binding molecules (e.g., an immune receptors, antibodies or functional fragments thereof). In some embodiments, the partitioned particle (e.g., biological particle) is a cell, cell nucleus, or cell bead of a cell of B-cell lineage (e.g., a plasma cell or memory B cell) which expresses an antigen-binding molecule, e.g., a variant antigen-binding molecule described herein.

The term “partition,” as used herein, generally, refers to a space or volume that can be suitable to contain one or more biological particle (e.g., cells, cell beads, or nuclei), one or more species of features or compounds, or conduct one or more reactions. A partition can be a physical container, compartment, or vessel, such as a droplet, a flow cell, a reaction chamber, a reaction compartment, a tube, a well, or a microwell. In some embodiments, the compartments or partitions include partitions that are flowable within fluid streams. These partitions can include, for example, micro-vesicles that have an outer barrier surrounding an inner fluid center or core, or, in some cases, the partitions can include a porous matrix that is capable of entraining and/or retaining materials within its matrix. In some aspects, partitions comprise droplets of aqueous fluid within a non-aqueous continuous phase (e.g., oil phase). A variety of different vessels are described in, for example, U.S. Patent Application Publication No. 2014/0155295. Emulsion systems for creating stable droplets in non-aqueous or oil continuous phases are described in detail in, e.g., U.S. Patent Application Publication No. 2010/010511.

In some embodiments, a partition herein includes a space or volume that can be suitable to contain one or more species or conduct one or more reactions. A partition can be a physical compartment, such as a droplet or well. The partition can be an isolated space or volume from another space or volume. The droplet can be a first phase (e.g., aqueous phase) in a second phase (e.g., oil) immiscible with the first phase. The droplet can be a first phase in a second phase that does not phase separate from the first phase, such as, for example, a capsule or liposome in an aqueous phase. A partition can include one or more other (inner) partitions. In some cases, a partition can be a virtual compartment that can be defined and identified by an index (e.g., indexed libraries) across multiple and/or remote physical compartments. For example, a physical compartment can include a plurality of virtual compartments.

In some embodiments, the methods and system described herein provide for the compartmentalization, depositing or partitioning of individual biological particles (e.g., cells, cell beads, or nuclei) from a sample material containing the biological particles (e.g., cells, cell beads, or nuclei) into discrete partitions, where each partition maintains separation of its own contents from the contents of other partitions. Identifiers including unique identifiers (e.g., UMI) and common or universal tags, e.g., barcodes, can be previously, subsequently or concurrently delivered to the partitions that hold the compartmentalized or partitioned biological particles (e.g., cells, cell beads, or nuclei), in order to allow for the later attribution of the characteristics of the individual biological particles (e.g., cells, cell beads, or nuclei) to one or more particular compartments. Further, identifiers including unique identifiers and common or universal tags, e.g., barcodes, can be coupled to labelling agents and previously, subsequently or concurrently delivered to the partitions that hold the compartmentalized or partitioned biological particles (e.g., cells, cell beads, or nuclei), in order to allow for the later attribution of the characteristics of the individual biological particles (e.g., cells, cell beads, or nuclei) to one or more particular compartments. Identifiers including unique identifiers and common or universal tags, e.g., barcodes, can be delivered, for example on an oligonucleotide, to a partition via any suitable mechanism, for example by coupling the barcoded oligonucleotides to a bead. In some embodiments, the barcoded oligonucleotides are reversibly (e.g., releasably) coupled to a bead. The bead suitable for the compositions and methods of the disclosure can have different surface chemistries and/or physical volumes. In some embodiments, the bead includes a polymer gel. In some embodiments, the polymer gel is a polyacrylamide. Additional non-limiting examples of suitable beads include microparticles, nanoparticles, beads, and microbeads. The partition can be a droplet in an emulsion. A partition can include one or more particles. A partition can include one or more types of particles. For example, a partition of the present disclosure can include one or more biological particles, e.g., labelled engineered cells, B cells, or memory B cells, organelles (e.g., nuclei), and/or macromolecular constituents thereof. A partition can include one or more gel beads. A partition can include one or more cell beads. A partition can include a single gel bead, a single cell bead, or both a single cell bead and single gel bead. A partition can include one or more reagents. Alternatively, a partition can be unoccupied. For example, a partition cannot comprise a bead. Unique identifiers, such as barcodes, can be injected into the droplets previous to, subsequent to, or concurrently with droplet generation, such as via a bead, as described elsewhere herein. Microfluidic channel networks (e.g., on a chip) can be utilized to generate partitions as described herein. Alternative mechanisms can also be employed in the partitioning of individual biological particles (e.g., cells, cell beads, or nuclei), including porous membranes through which aqueous mixtures of cells are extruded into non-aqueous fluids.

The partitions can be flowable within fluid streams. The partitions can include, for example, micro-vesicles that have an outer barrier surrounding an inner fluid center or core. In some cases, the partitions can include a porous matrix that is capable of entraining and/or retaining materials (e.g., expressed antibodies or antigen-binding fragments thereof) within its matrix (e.g., via a capture agent configured to couple to both the matrix and the expressed antibody or antigen-binding fragment thereof). The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions can be provided in a water-in-oil emulsion or oil-in-water emulsion. A variety of different vessels are described in, for example, U.S. Patent Application Publication No. 2014/0155295. Emulsion systems for creating stable droplets in non-aqueous or oil continuous phases are described in, for example, U.S. Patent Application Publication No. 2010/0105112.

In the case of droplets in an emulsion, allocating individual biological particles (e.g., cells, cell beads, nuclei, or labelled engineered cells) to discrete partitions can, in one non-limiting example, be accomplished by introducing a flowing stream of biological particles (e.g., cells, cell beads, nuclei, or labelled engineered cells) in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters can be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions can contain less than one biological particle per partition in order to ensure that those partitions that are occupied are primarily singly occupied. In some cases, partitions among a plurality of partitions can contain at most one biological particle (e.g., bead, DNA, nucleus, cell, such as a labelled engineered cells, B cells, or memory B cells, or cellular/nuclear material). In some embodiments, the various parameters (e.g., fluid properties, particle properties, microfluidic architectures, etc.) can be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

In some embodiments, the method further includes individually partitioning one or more single biological particles (e.g., cells, such as engineered cells, B cells, cell beads, or nuclei) from a plurality of biological particles (e.g., cells, such as engineered cells, B cells, cell beads, or nuclei) in a partition of a second plurality of partitions.

In some embodiments, at least one of the first and second plurality of partitions includes a microwell, a flow cell, a reaction chamber, a reaction compartment, or a droplet. In some embodiments, at least one of the first and second plurality of partitions includes individual droplets in emulsion. In some embodiments, the partitions of the first plurality and/or the second plurality of partition have the same reaction volume.

In the case of droplets in emulsion, allocating individual biological particles (e.g., cells, cell beads, or nuclei) to discrete partitions can generally be accomplished by introducing a flowing stream of cells in an aqueous fluid into a flowing stream of a non-aqueous fluid, such that droplets are generated at the junction of the two streams. By providing the aqueous cell-containing stream at a certain concentration of cells, the occupancy of the resulting partitions (e.g., number of biological particles, such as cells, cell beads, or nuclei, per partition) can be controlled. For example, where single cell partitions are desired, the relative flow rates of the fluids can be selected such that, on average, the partitions contain less than one cell per partition, in order to ensure that those partitions that are occupied, are primarily singly occupied. In some embodiments, the relative flow rates of the fluids can be selected such that a majority of partitions are occupied, e.g., allowing for only a small percentage of unoccupied partitions. In some embodiments, the flows and channel architectures are controlled as to ensure a desired number of singly occupied partitions, less than a certain level of unoccupied partitions and less than a certain level of multiply occupied partitions.

In some embodiments, the methods described herein can be performed such that a majority of occupied partitions include no more than one biological particle (e.g., cell, cell bead, or nucleus) per occupied partition. In some embodiments, the partitioning process is performed such that fewer than 25%, fewer than 20%, fewer than 15%, fewer than 10%, fewer than 5%, fewer than 2%, or fewer than 1% the occupied partitions contain more than one biological particle (e.g., cell, cell bead, or nucleus). In some embodiments, fewer than 20% of the occupied partitions include more than one biological particle (e.g., cell, cell bead, or nucleus). In some embodiments, fewer than 10% of the occupied partitions include more than one biological particle (e.g., cell, cell bead, or nucleus) per partition. In some embodiments, fewer than 5% of the occupied partitions include more than one cell per partition. In some embodiments, it is desirable to avoid the creation of excessive numbers of empty partitions. For example, from a cost perspective and/or efficiency perspective, it may be desirable to minimize the number of empty partitions. While this can be accomplished by providing sufficient numbers of biological particles (e.g., cells or nuclei) into the partitioning zone, the Poissonian distribution can optionally be used to increase the number of partitions that include multiple biological particles (e.g., cells or nuclei). As such, in some embodiments described herein, the flow of one or more of the biological particles (e.g., cells or nuclei), or other fluids directed into the partitioning zone are performed such that no more than 50% of the generated partitions, no more than 25% of the generated partitions, or no more than 10% of the generated partitions are unoccupied. Further, in some aspects, these flows are controlled so as to present non-Poissonian distribution of single occupied partitions while providing lower levels of unoccupied partitions. Restated, in some aspects, the above noted ranges of unoccupied partitions can be achieved while still providing any of the single occupancy rates described above. For example, in some embodiments, the use of the systems and methods described herein creates resulting partitions that have multiple occupancy rates of less than 25%, less than 20%, less than 15%), less than 10%, and in some embodiments, less than 5%, while having unoccupied partitions of less than 50%), less than 40%, less than 30%, less than 20%, less than 10%, and in some embodiments, less than 5%.

Although described in terms of providing substantially singly occupied partitions, above, in some embodiments, the methods as described herein include providing multiply occupied partitions, e.g., containing two, three, four or more cells and/or beads comprising nucleic acid barcode molecules within a single partition.

In some embodiments, the reporter oligonucleotides contained within a partition are distinguishable from the reporter oligonucleotides contained within other partitions of the plurality of partitions. This can be accomplished by incorporating one or more partition-specific barcode sequences into the reporter barcode sequence of the reporter oligonucleotides contained within the partition.

In some embodiments, it may be desirable to incorporate multiple different barcode sequences within a given partition, either attached to a single or multiple beads within the partition. For example, in some cases, a mixed, but known barcode sequences set can provide greater assurance of identification in the subsequent processing, e.g., by providing a stronger address or attribution of the barcodes to a given partition, as a duplicate or independent confirmation of the output from a given partition.

### Microfluidic Channel Structures

Microfluidic channel networks (e.g., on a chip) can be utilized to generate partitions as described herein. Alternative mechanisms can also be employed in the partitioning of individual biological particles (e.g., cells, cell beads, or nuclei), including porous membranes through which aqueous mixtures of cells are extruded into non-aqueous fluids.

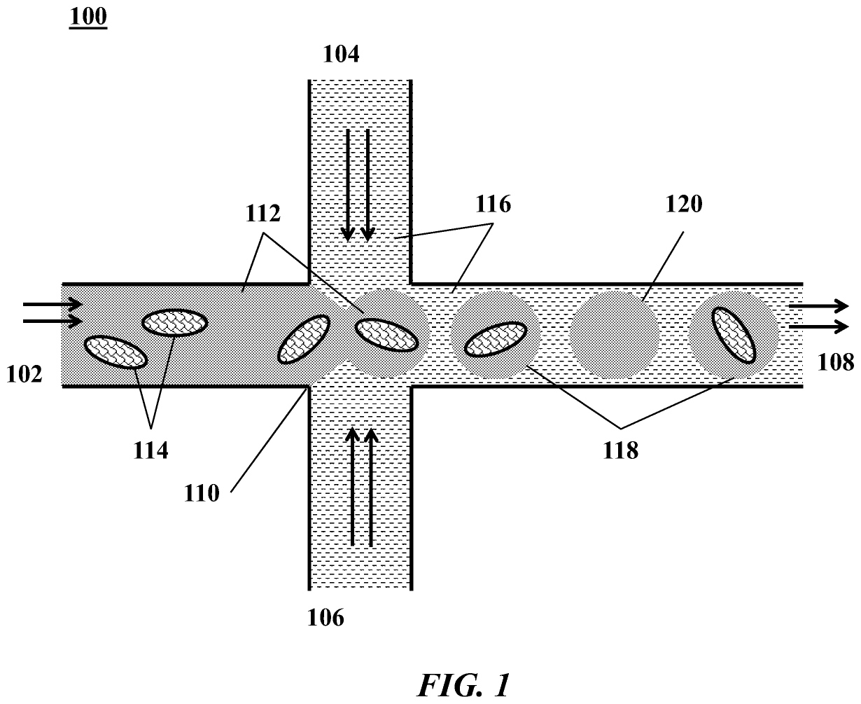

FIG. 1 shows an example of a microfluidic channel structure 100 for partitioning individual biological particles (e.g., cells, cell beads, or nuclei). The channel structure 100 can include channel segments 102, 104, 106 and 108 communicating at a channel junction 110. In operation, a first aqueous fluid 112 that includes suspended biological particles (e.g., cells such as labelled engineered cells, B cells, e.g., memory B cells, plasma cells, or nuclei) 114 can be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 can be fluidically coupled to an outlet reservoir where the discrete droplets can be stored and/or harvested. A discrete droplet generated can include an individual biological particle (e.g., a cell, a cell bead, or a nucleus) 114 (such as droplets 118). A discrete droplet generated can include more than one individual biological particle (e.g., a cell, a labelled engineered cell, a cell bead, or a nucleus) 114 (not shown in FIG. 1). A discrete droplet can contain no biological particle (e.g., a cell, a cell bead, or a nucleus) 114 (such as droplet 120). Each discrete partition can maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112.

As will be appreciated, the channel segments described herein can be coupled to any of a variety of different fluid sources or receiving components, including reservoirs, tubing, manifolds, or fluidic components of other systems. As will be appreciated, the microfluidic channel structure 100 can have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid can be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

The generated droplets can include two subsets of droplets: (1) occupied droplets 118, containing one or more biological particles 114, e.g., labelled engineered cells, B cells such as memory B cells, plasma cells, cell beads, or nuclei, and (2) unoccupied droplets 120, not containing any biological particles 114. Occupied droplets 118 can include singly occupied droplets (having one biological particle, such as one B cell, e.g., memory B cell, plasma cell, memory B cell, a cell bead or a nucleus) and multiply occupied droplets (having more than one biological particle, such as multiple B cells, memory B cells, or plasma cells). As described elsewhere herein, in some cases, the majority of occupied partitions can include no more than one biological particle, e.g., labelled engineered cells, B cells, memory B cells, plasma cells, cell beads, or nuclei, per occupied partition and some of the generated partitions can be unoccupied (of any biological particle, or labelled engineered cells, B cells, memory B cells, plasma cells, cell beads, or nuclei). In some cases, though, some of the occupied partitions can include more than one biological particle (e.g., labelled engineered cells, B cells, memory B cells, plasma cells, cell beads, or nuclei). In some cases, the partitioning process can be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle (e.g., cell, cell bead, or nucleus) per partition.

In some cases, it can be desirable to minimize the creation of excessive numbers of empty partitions, such as to reduce costs and/or increase efficiency. While this minimization can be achieved by providing a sufficient number of biological particles (e.g., biological particles, such as labelled engineered cells, B cells, memory B cells, plasma cells, cell beads, or nuclei 114) at the partitioning junction 110, such as to ensure that at least one biological particle (e.g., cell, labelled engineered cell, B cell, memory B cell, plasma cell, cell bead, or nucleus) is encapsulated in a partition, the Poissonian distribution can expectedly increase the number of partitions that include multiple biological particles (e.g., cells, labelled engineered cells, B cells, memory B cells, plasma cells, cell beads, or nuclei). As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles, such as B cells e.g., memory B cells, or plasma cells, (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

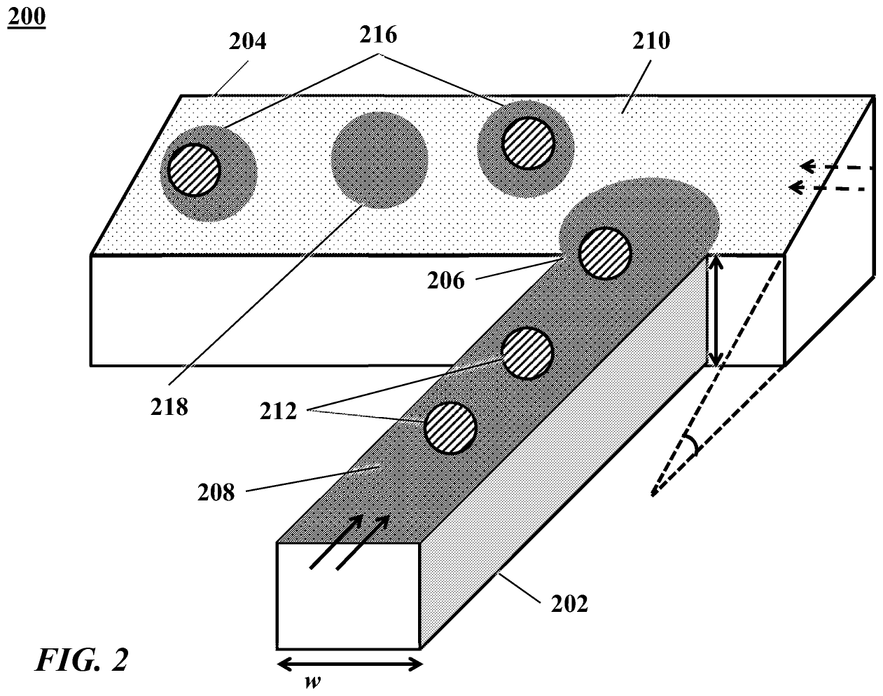

As will be appreciated, the above-described occupancy rates are also applicable to partitions that include both biological particles (e.g., cells, labelled engineered cells, B cells, memory B cells, or nuclei) and additional reagents, including, but not limited to, beads (e.g., gel beads) carrying nucleic acid barcode molecules (e.g., barcoded oligonucleotides) (described in relation to FIGS. 1 and 2). The occupied partitions (e.g., at least about 10%, 20%, 30%, 40%, 50%, 60%, 70%, 80%, 90%, 95%, or 99% of the occupied partitions) can include both a bead comprising nucleic acid barcode molecules and a biological particle (e.g., a cell, a B cell, a memory B cell, or a nucleus).

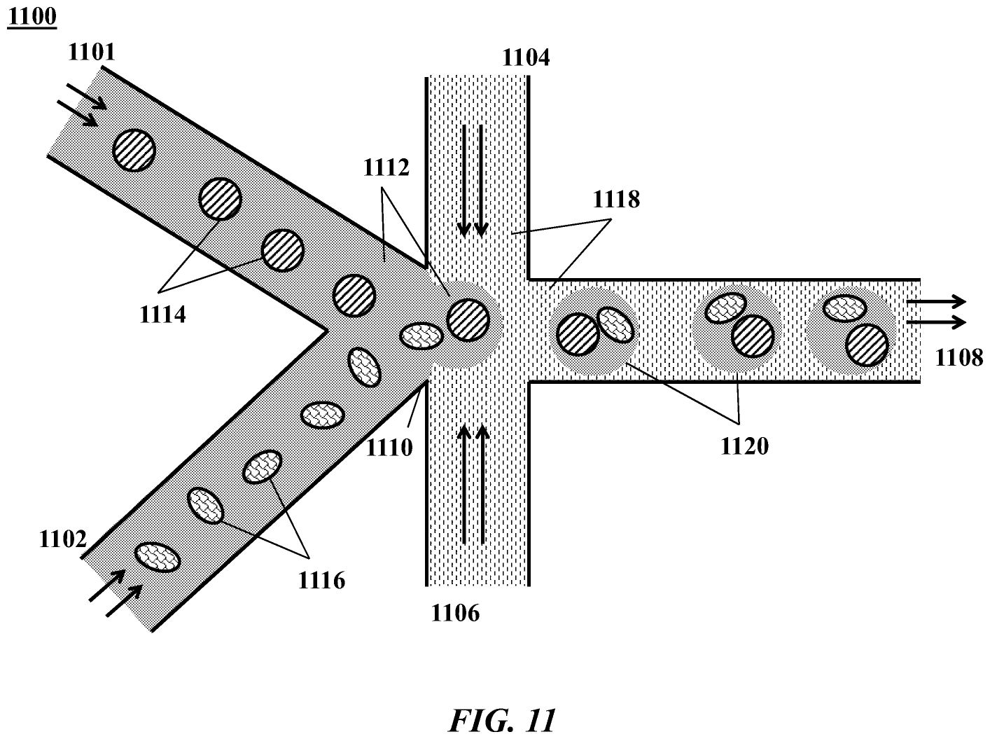

FIG. 11 shows an example of a microfluidic channel structure 1100 for delivering barcode carrying beads to droplets. The channel structure 1100 can include channel segments 1101, 1116, 1104, 1106 and 1108 communicating at a channel junction 1110. In operation, the channel segment 1101 may transport an aqueous fluid 1112 that includes a plurality of beads 1114 (e.g., with nucleic acid molecules, oligonucleotides, molecular tags) along the channel segment 1101 into junction 1110. The plurality of beads 1114 may be sourced from a suspension of beads. For example, the channel segment 1101 may be connected to a reservoir comprising an aqueous suspension of beads 1114. The channel segment 1102 may transport the aqueous fluid 1112 that includes a plurality of biological particles (e.g., cells or nuclei) 1116 along the channel segment 1102 into junction 1110. The plurality of biological particles 1116 may be sourced from a suspension of biological particles (e.g., cells or nuclei). For example, the channel segment 1102 may be connected to a reservoir comprising an aqueous suspension of biological particles (e.g., cells or nuclei) 1116. In some instances, the aqueous fluid 1112 in either the first channel segment 1101 or the second channel segment 1102, or in both segments, can include one or more reagents, as further described below. A second fluid 1118 that is immiscible with the aqueous fluid 1112 (e.g., oil) can be delivered to the junction 1110 from each of channel segments 1104 and 1106. Upon meeting of the aqueous fluid 1112 from each of channel segments 1101 and 1102 and the second fluid 1118 from each of channel segments 1104 and 1106 at the channel junction 1110, the aqueous fluid 1112 can be partitioned as discrete droplets 1120 in the second fluid 1118 and flow away from the junction 1110 along channel segment 1108. The channel segment 1108 may deliver the discrete droplets to an outlet reservoir fluidly coupled to the channel segment 1108, where they may be harvested. As an alternative, the channel segments 1101 and 1102 may meet at another junction upstream of the junction 1110. At such junction, beads and biological particles (e.g., cells or nuclei) may form a mixture that is directed along another channel to the junction 1110 to yield droplets 1120. The mixture may provide the beads and biological particles in an alternating fashion, such that, for example, a droplet comprises a single bead and a single biological particle (e.g., cell or nucleus).

In another aspect, in addition to or as an alternative to droplet-based partitioning, biological particles (e.g., cells) may be encapsulated within a particulate material to form a “cell bead”.

The cell bead can include other reagents. Encapsulation of biological particles (e.g., B cells, labelled engineered cells, or nuclei), can be performed by a variety of processes. Such processes can combine an aqueous fluid containing the biological particles (e.g., cells or nuclei) with a polymeric precursor material that can be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Encapsulation of biological particles, e.g., labelled engineered cells, B cells, e.g., memory B cells, plasma cells, or nuclei, can be performed by a variety of methods. For example, air knife droplet or aerosol generators may be used to dispense droplets of precursor fluids into gelling solutions in order to form cell beads that include individual biological particles or small groups of biological particles (e.g., cells or nuclei). Likewise, membrane-based encapsulation systems may be used to generate cell beads comprising encapsulated biological particles as described herein. Microfluidic systems of the present disclosure, such as that shown in FIG. 1, may be readily used in encapsulating biological particles (e.g., cells, B cells, memory B cells, or nuclei) as described herein. Exemplary methods for encapsulating biological particles (e.g., cells, B cells, memory B cells, or nuclei) are also further described in U.S. Patent Application Pub. No. US 2015/0376609 and PCT/US2018/016019. In particular, and with reference to FIG. 1, the aqueous fluid 112 comprising (i) the biological particles (e.g., cells, B cells, memory B cells, or nuclei) 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the bead that includes the entrained biological particles (e.g., cells, B cells, memory B cells, or nuclei). Examples of polymer precursor/initiator pairs include those described in U.S. Patent Application Publication No. 2014/0378345.

For example, in the case where the polymer precursor material comprises a linear polymer material, such as a linear polyacrylamide, PEG, or other linear polymeric material, the activation agent can include a cross-linking agent, or a chemical that activates a cross-linking agent within the formed droplets. Likewise, for polymer precursors that comprise polymerizable monomers, the activation agent can include a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) can be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED can diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) cell beads, as solid or semi-solid beads or particles entraining the biological particles, (e.g., cells, such as labelled engineered cells, B cells, memory B cells, or nuclei) 114. Although described in terms of polyacrylamide encapsulation, other “activatable” encapsulation compositions can also be employed in the context of the methods and compositions described herein. For example, formation of alginate droplets followed by exposure to divalent metal ions (e.g., Ca²⁺ ions), can be used as an encapsulation process using the described processes. Likewise, agarose droplets can also be transformed into capsules through temperature based gelling (e.g., upon cooling, etc.).

In some cases, encapsulated biological particles (e.g., cells, labelled engineered cells, B cells, memory B cells, or nuclei) can be selectively releasable from the cell bead, such as through passage of time or upon application of a particular stimulus, that degrades the encapsulating material sufficiently to allow the biological particles (e.g., labelled engineered including B cells or nuclei), or its other contents to be released from the encapsulating material, such as into a partition (e.g., droplet). For example, in the case of the polyacrylamide polymer described above, degradation of the polymer can be accomplished through the introduction of an appropriate reducing agent, such as DTT or the like, to cleave disulfide bonds that cross-link the polymer matrix. See, for example, U.S. Patent Application Publication No. 2014/0378345.

The biological particle (e.g., labelled cells, such as labelled engineered cells, B cells or nuclei), can be subjected to other conditions sufficient to polymerize or gel the precursors. The conditions sufficient to polymerize or gel the precursors can include exposure to heating, cooling, electromagnetic radiation, and/or light. The conditions sufficient to polymerize or gel the precursors can include any conditions sufficient to polymerize or gel the precursors. Following polymerization or gelling, a polymer or gel can be formed around the biological particle (e.g., labelled cells, such as labelled engineered cells, B cells or nuclei). The polymer or gel can be diffusively permeable to chemical or biochemical reagents. The polymer or gel can be diffusively impermeable to macromolecular constituents (e.g., secreted antibodies or antigen-binding fragments thereof) of the biological particle (e.g., labelled cells such as labelled engineered cells, B cells, or nuclei). In this manner, the polymer or gel can act to allow the biological particle (e.g., labelled cells such as labelled engineered cells, B cells, or nuclei) to be subjected to chemical or biochemical operations while spatially confining the macromolecular constituents to a region of the droplet defined by the polymer or gel. The polymer or gel can include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel can include any other polymer or gel.

The polymer or gel can be functionalized (e.g., coupled to a capture agent) to bind to targeted analytes (e.g., secreted antibodies or antigen-binding fragment thereof), such as nucleic acids, proteins, carbohydrates, lipids or other analytes. The polymer or gel can be polymerized or gelled via a passive mechanism. The polymer or gel can be stable in alkaline conditions or at elevated temperature. The polymer or gel can have mechanical properties similar to the mechanical properties of the bead. For instance, the polymer or gel can be of a similar size to the bead. The polymer or gel can have a mechanical strength (e.g., tensile strength) similar to that of the bead. The polymer or gel can be of a lower density than an oil. The polymer or gel can be of a density that is roughly similar to that of a buffer. The polymer or gel can have a tunable pore size. The pore size can be chosen to, for instance, retain denatured nucleic acids. The pore size can be chosen to maintain diffusive permeability to exogenous chemicals such as sodium hydroxide (NaOH) and/or endogenous chemicals such as inhibitors. The polymer or gel can be biocompatible. The polymer or gel can maintain or enhance cell viability. The polymer or gel can be biochemically compatible. The polymer or gel can be polymerized and/or depolymerized thermally, chemically, enzymatically, and/or optically.

The polymer can include poly(acrylamide-co-acrylic acid) crosslinked with disulfide linkages. The preparation of the polymer can include a two-step reaction. In the first activation step, poly(acrylamide-co-acrylic acid) can be exposed to an acylating agent to convert carboxylic acids to esters. For instance, the poly(acrylamide-co-acrylic acid) can be exposed to 4-(4,6-dimethoxy-1,3,5-triazin-2-yl)-4-methylmorpholinium chloride (DMTMM). The polyacrylamide-co-acrylic acid can be exposed to other salts of 4-(4,6-dimethoxy-1,3,5-triazin-2-yl)-4-methylmorpholinium. In the second cross-linking step, the ester formed in the first step can be exposed to a disulfide crosslinking agent. For instance, the ester can be exposed to cystamine (2,2′-dithiobis(ethylamine)). Following the two steps, the biological particle (e.g., the labelled biological particle, including a labelled cell or nucleus) can be surrounded by polyacrylamide strands linked together by disulfide bridges. In this manner, the biological particle can be encased inside of or comprise a gel or matrix (e.g., polymer matrix) to form a “cell bead.” A cell bead can contain biological particles (e.g., labelled cells, such as labelled engineered cells, B cells, or nuclei) or macromolecular constituents (e.g., RNA, DNA, proteins, secreted antibodies or antigen-binding fragments thereof etc.) of biological particles. A cell bead can include a single cell/nucleus or multiple cells/nuclei, or a derivative of the single cell/nucleus or multiple cells/nuclei. For example after lysing and washing the cells, inhibitory components from cell or nucleus lysates can be washed away and the macromolecular constituents can be bound as cell beads. Systems and methods disclosed herein can be applicable to both (i) cell beads (and/or droplets or other partitions) containing biological particles and (ii) cell beads (and/or droplets or other partitions) containing macromolecular constituents of biological particles.

Encapsulated biological particles (e.g., labelled cells, such as labelled engineered cells, B cells, or nuclei) can provide certain potential advantages of being more storable and more portable than droplet-based partitioned biological particles. Furthermore, in some cases, it can be desirable to allow biological particles (e.g., labelled cells, such as labelled engineered cells, B cells, or nuclei) to incubate for a select period of time before analysis, such as in order to characterize changes in such biological particles over time, either in the presence or absence of different stimuli (e.g., cytokines, antigens, etc.). In such cases, encapsulation can allow for longer incubation than partitioning in emulsion droplets, although in some cases, droplet partitioned biological particles can also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles (e.g., labelled cells, such as labelled engineered cells, B cells, or nuclei) can constitute the partitioning of the biological particles into which other reagents are co-partitioned. Alternatively or in addition, encapsulated biological particles can be readily deposited into other partitions (e.g., droplets) as described above.

### Microwells

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well can be configured to hold a volume of about 1000 μL, about 100 μL, about 10 μL, about 1 μL, about 100 nL, about 10 nL, about 1 nL, about 100 pL, about 10 pL, etc. The well can be configured to hold a volume of at least 10 pL, at least 100 pL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 pL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a biological particle (e.g., cell, cell nucleus, cell bead), or other sample (e.g., cellular components, nucleic acid molecules, etc.) while the adjacent microwell can be used to contain a droplet, bead, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, and combinations thereof. The reagents can be physically separated from a sample (for example, a cell, nucleus, cell bead, or cellular components, e.g., proteins, nucleic acid molecules, etc.) that is placed in the well. This physical separation can be accomplished by containing the reagents within, or coupling to, a bead that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

A well can include free reagents and/or reagents encapsulated in, or otherwise coupled to or associated with, beads or droplets. In some embodiments, any of the reagents described in this disclosure can be encapsulated in, or otherwise coupled to, a droplet or bead, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells disclosed herein can be provided as a part of a kit. For example, a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for cell lysis, fixation, and/or permeabilization).

In some cases, a well includes a bead or droplet that includes a set of reagents that has a similar attribute, for example, a set of enzymes, a set of minerals, a set of oligonucleotides, a mixture of different barcode molecules, a mixture of identical barcode molecules. In other cases, a bead or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

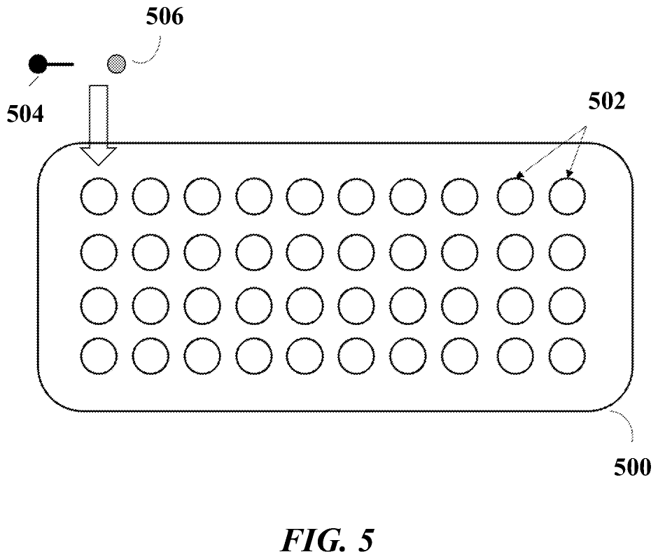

A non-limiting example of a microwell array in accordance with some embodiments of the disclosure is schematically presented in FIG. 5. In this example, the array can be contained within a substrate 500. The substrate 500 includes a plurality of wells 502. The wells 502 can be of any size or shape, and the spacing between the wells, the number of wells per substrate, as well as the density of the wells on the substrate 500 can be modified, depending on the particular application. In one such example application, a sample molecule 506, which can include a biological particle (e.g., a cell/nucleus) or cellular/nuclear components (e.g., nucleic acid molecules) is co-partitioned with a bead 504, which can include a nucleic acid barcode molecule coupled thereto. The wells 502 can be loaded using gravity or other loading technique (e.g., centrifugation, liquid handler, acoustic loading, optoelectronic, etc.). In some instances, at least one of the wells 502 contains a single sample molecule 506 (e.g., cell, cell bead, or nucleus) and a single bead 504.

Reagents can be loaded into a well either sequentially or concurrently. In some cases, reagents are introduced to the device either before or after a particular operation. In some cases, reagents (which can be provided, in certain instances, in droplets or beads) are introduced sequentially such that different reactions or operations occur at different steps. The reagents (or droplets, or beads) can also be loaded at operations interspersed with a reaction or operation step. For example, or droplets or beads including reagents for fragmenting polynucleotides (e.g., restriction enzymes) and/or other enzymes (e.g., transposases, ligases, polymerases, etc.) can be loaded into the well or plurality of wells, followed by loading of droplets or beads including reagents for attaching nucleic acid barcode molecules to a sample nucleic acid molecule. Reagents can be provided concurrently or sequentially with a sample, e.g., a cell/nucleus or cellular/nuclear components (e.g., organelles, proteins, nucleic acid molecules, carbohydrates, lipids, etc.). Accordingly, use of wells can be useful in performing multi-step operations or reactions.

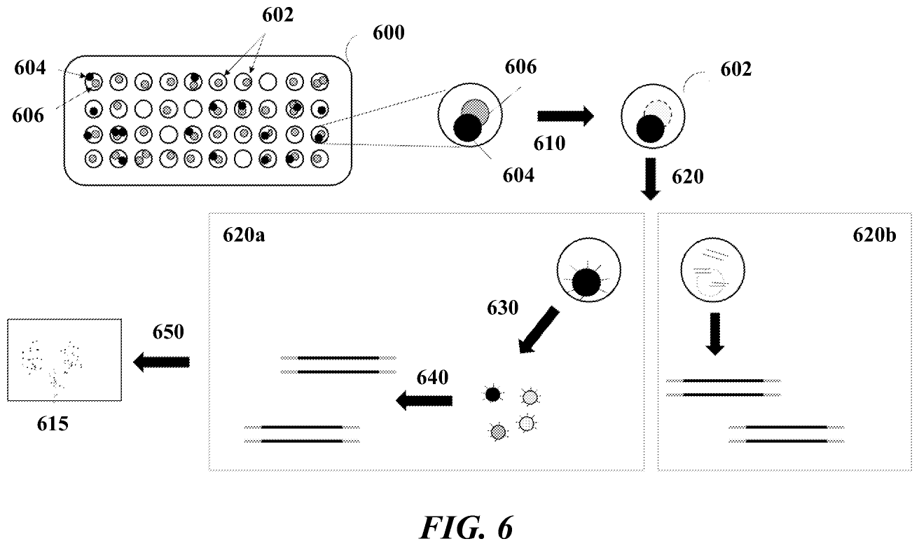

FIG. 6 schematically shows an example workflow for processing nucleic acid molecules within a sample. A substrate 600 including a plurality of microwells 602 can be provided. A sample 606 which can include a cell, cell bead, cellular components or analytes (e.g., proteins and/or nucleic acid molecules) can be co-partitioned, in a plurality of microwells 602, with a plurality of beads 604 including nucleic acid barcode molecules. During a partitioning process, the sample 606 can be processed within the partition. For instance, in the case of live cells, the cell can be subjected to conditions sufficient to lyse the cells and release the analytes contained therein. In process 620, the bead 604 can be further processed. By way of example, processes 620a and 620b schematically illustrate different workflows, depending on the properties of the bead 604.

In 620a, the bead includes nucleic acid barcode molecules that are attached thereto, and sample nucleic acid molecules (e.g., RNA, DNA) can attach, e.g., via hybridization of ligation, to the nucleic acid barcode molecules. Such attachment can occur on the bead. In process 630, the beads 604 from multiple wells 602 can be collected and pooled. Further processing can be performed in process 640. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 650, further characterization, such as sequencing can be performed to generate sequencing reads. The sequencing reads can yield information on individual cells or populations of cells, which can be represented visually or graphically, e.g., in a plot.

In 620b, the bead includes nucleic acid barcode molecules that are releasably attached thereto, as described below. The bead can degrade or otherwise release the nucleic acid barcode molecules into the well 602; the nucleic acid barcode molecules can then be used to barcode nucleic acid molecules within the well 602. Further processing can be performed either inside the partition or outside the partition. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 650, further characterization, such as sequencing can be performed to generate sequencing reads. The sequencing reads can yield information on individual cells or populations of cells, which can be represented visually or graphically, e.g., in a plot.

As described elsewhere herein, the nucleic acid barcode molecules and other reagents can be contained within a bead or droplet. These beads or droplets can be loaded into a partition (e.g., a microwell) before, after, or concurrently with the loading of a biological particle (e.g., cell, cell bead, or nucleus), such that each biological particle is contacted with a different bead or droplet. This technique can be used to attach a unique nucleic acid barcode molecule to nucleic acid molecules obtained from each biological particle (e.g., cell, cell bead, or nucleus). Alternatively or in addition, the sample nucleic acid molecules can be attached to a support. For example, the partition (e.g., microwell) can include a bead which has coupled thereto a plurality of nucleic acid barcode molecules. The sample nucleic acid molecules, or derivatives thereof, can couple or attach to the nucleic acid barcode molecules attached on the support. The resulting barcoded nucleic acid molecules can then be removed from the partition, and in some instances, pooled and sequenced. In such cases, the nucleic acid barcode sequences can be used to trace the origin of the sample nucleic acid molecule. For example, polynucleotides with identical barcodes can be determined to originate from the same biological particle (e.g., cell, cell bead, or nucleus) or partition, while polynucleotides with different barcodes can be determined to originate from different biological particles (e.g., cells cell beads, or nuclei) or partitions.

The samples or reagents can be loaded in the wells or microwells using a variety of approaches. For example, the samples (e.g., a cell, nucleus, cell bead, or cellular/nuclear component) or reagents (as described herein) can be loaded into the well or microwell using an external force, e.g., gravitational force, electrical force, magnetic force, or using mechanisms to drive the sample or reagents into the well, for example, via pressure-driven flow, centrifugation, optoelectronics, acoustic loading, electrokinetic pumping, vacuum, capillary flow, etc. In certain cases, a fluid handling system can be used to load the samples or reagents into the well. The loading of the samples or reagents can follow a Poissonian distribution or a non-Poissonian distribution, e.g., super Poisson or sub-Poisson. The geometry, spacing between wells, density, and size of the microwells can be modified to accommodate a useful sample or reagent distribution; for example, the size and spacing of the microwells can be adjusted such that the sample or reagents can be distributed in a super-Poissonian fashion.

In one non-limiting example, the microwell array or plate includes pairs of microwells, in which each pair of microwells is configured to hold a droplet (e.g., including a single biological particle, such as a cell, cell bead, or nucleus) and a single bead (such as those described herein, which can, in some instances, also be encapsulated in a droplet). The droplet and the bead (or droplet containing the bead) can be loaded simultaneously or sequentially, and the droplet and the bead can be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets including different reagents and/or samples, which are merged upon contact or upon application of a stimulus. In such instances, the droplet of one microwell of the pair can include reagents that can react with an agent in the droplet of the other microwell of the pair. For example, one droplet can include reagents that are configured to release the nucleic acid barcode molecules of a bead contained in another droplet, located in the adjacent microwell. Upon merging of the droplets, the nucleic acid barcode molecules can be released from the bead into the partition (e.g., the microwell or microwell pair that are in contact), and further processing can be performed (e.g., barcoding, nucleic acid reactions, etc.). In cases where intact or live cells are loaded in the microwells, one of the droplets can include lysis reagents for lysing the cell upon droplet merging.

In some embodiments, a droplet or bead can be partitioned into a well. The droplets can be selected or subjected to pre-processing prior to loading into a well. For instance, the droplets can include biological particles (e.g., cells, cell beads or nuclei), and only certain droplets, such as those containing a single biological particle (or at least one biological particle), can be selected for use in loading of the wells. Such a pre-selection process can be useful in efficient loading of single biological particles (e.g, cells, cell beads, or nuclei), such as to obtain a non-Poissonian distribution, or to pre-filter cells for a selected characteristic prior to further partitioning in the wells. Additionally, the technique can be useful in obtaining or preventing biological particle (e.g., cell, cell bead, or nucleus) doublet or multiplet formation prior to or during loading of the microwell.

In some embodiments, the wells can include nucleic acid barcode molecules attached thereto. The nucleic acid barcode molecules can be attached to a surface of the well (e.g., a wall of the well). The nucleic acid barcode molecules may be attached to a droplet or bead that has been partitioned into the well. The nucleic acid barcode molecule (e.g., a partition barcode sequence) of one well can differ from the nucleic acid barcode molecule of another well, which can permit identification of the contents contained with a single partition or well. In some embodiments, the nucleic acid barcode molecule can include a spatial barcode sequence that can identify a spatial coordinate of a well, such as within the well array or well plate. In some embodiments, the nucleic acid barcode molecule can include a unique molecular identifier for individual molecule identification. In some instances, the nucleic acid barcode molecules can be configured to attach to or capture a nucleic acid molecule within a sample or biological particle (e.g., cell, cell bead, or nucleus) distributed in the well. For example, the nucleic acid barcode molecules can include a capture sequence that can be used to capture or hybridize to a nucleic acid molecule (e.g., RNA, DNA) within the sample. In some embodiments, the nucleic acid barcode molecules can be releasable from the microwell. In some instances, the nucleic acid barcode molecules may be releasable from the bead or droplet. For example, the nucleic acid barcode molecules can include a chemical cross-linker which can be cleaved upon application of a stimulus (e.g., photo-, magnetic, chemical, biological, stimulus). The released nucleic acid barcode molecules, which can be hybridized or configured to hybridize to a sample nucleic acid molecule, can be collected and pooled for further processing, which can include nucleic acid processing (e.g., amplification, extension, reverse transcription, etc.) and/or characterization (e.g., sequencing). In some instances nucleic acid barcode molecules attached to a bead or droplet in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences can be used to identify the biological particle (e.g., cell, cell bead or nucleus) or partition from which a nucleic acid molecule originated.

Characterization of samples within a well can be performed. Such characterization can include, in non-limiting examples, imaging of the sample (e.g., cell, nucleus, cell bead, or cellular/nuclear components) or derivatives thereof. Characterization techniques such as microscopy or imaging can be useful in measuring sample profiles in fixed spatial locations. For example, when biological particles (e.g., cells, cell beads, or nuclei) are partitioned, optionally with beads, imaging of each microwell and the contents contained therein can provide useful information on biological particles (e.g., cell, cell bead, or nucleus) doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging can be used to characterize live cells in the wells, including, but not limited to: dynamic live-cell tracking, cell-cell interactions (when two or more cells are co-partitioned), cell proliferation, etc. Alternatively or in addition to, imaging can be used to characterize a quantity of amplification products in the well.

In operation, a well can be loaded with a sample and reagents, simultaneously or sequentially. When biological particles (e.g., cells, nuclei or cell beads) are loaded, the well can be subjected to washing, e.g., to remove excess cells from the well, microwell array, or plate. Similarly, washing can be performed to remove excess beads or other reagents from the well, microwell array, or plate. In the instances where live cells are used, the cells can be lysed in the individual partitions to release the intracellular components or cellular analytes. Alternatively, the cells or nuclei can be fixed or permeabilized in the individual partitions. The intracellular components or cellular analytes can couple to a support, e.g., on a surface of the microwell, on a solid support (e.g., bead), or they can be collected for further downstream processing. For example, after cell or nucleus lysis, the intracellular components or cellular analytes can be transferred to individual droplets or other partitions for barcoding. Alternatively, or in addition, the intracellular components or cellular analytes (e.g., nucleic acid molecules) can couple to a bead including a nucleic acid barcode molecule; subsequently, the bead can be collected and further processed, e.g., subjected to nucleic acid reaction such as reverse transcription, amplification, or extension, and the nucleic acid molecules thereon can be further characterized, e.g., via sequencing. Alternatively, or in addition, the intracellular components or cellular analytes can be barcoded in the well (e.g., using a bead including nucleic acid barcode molecules that are releasable or on a surface of the microwell including nucleic acid barcode molecules). The barcoded nucleic acid molecules or analytes can be further processed in the well, or the barcoded nucleic acid molecules or analytes can be collected from the individual partitions and subjected to further processing outside the partition. Further processing can include nucleic acid processing (e.g., performing an amplification, extension) or characterization (e.g., fluorescence monitoring of amplified molecules, sequencing). At any suitable or useful step, the well (or microwell array or plate) can be sealed (e.g., using an oil, membrane, wax, etc.), which enables storage of the assay or selective introduction of additional reagents.

Once sealed, the well may be subjected to conditions for further processing of a biological particle (e.g., a cell, a cell bead or a nucleus) in the well. For instance, reagents in the well may allow further processing of the biological particle, e.g., lysis of the cell or nucleus, as further described herein. Alternatively, the well (or wells such as those of a well-based array) comprising the biological particle (e.g., cell, cell bead, or nucleus) may be subjected to freeze-thaw cycling to process the biological particle(s), e.g., lysis of a cell or nucleus. The well containing the biological particle (e.g., cell, cell bead, or nucleus) may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the biological particle(s) (e.g., cell(s), cell bead(s), nucleus or nuclei) may be subjected to freeze thaw cycles to lyse biological particle(s). In one embodiment, the initially frozen well (or wells) are thawed to a temperature above freezing (e.g., room temperature or 25° C.). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, or 4 times, to obtain lysis of the biological particle(s) (e.g., cell(s), cell bead(s), nucleus, or nuclei) in the well (or wells). In one embodiment, the freezing, thawing and/or freeze/thaw cycling is performed in the absence of a lysis buffer.

### Bead

In some embodiments of the disclosure, a partition can include one or more unique identifiers, such as barcodes (e.g., a plurality of nucleic acid barcode molecules which can be, for example, a plurality of partition barcode sequences). Barcodes can be previously, subsequently or concurrently delivered to the partitions that hold the compartmentalized or partitioned biological particles (e.g., labelled cells such as engineered cells, B cells, or nuclei). For example, barcodes can be injected into droplets previous to, subsequent to, or concurrently with droplet generation. In some embodiments, the delivery of the barcodes to a particular partition allows for the later attribution of the characteristics of the individual biological particle (e.g., labelled cells such as engineered cells, B cells, or nuclei) to the particular partition. Barcodes can be delivered, for example on a nucleic acid barcode molecule (e.g., a barcoded oligonucleotide), to a partition via any suitable mechanism. In some embodiments, nucleic acid barcode molecules can be delivered to a partition via a bead. Beads are described in further detail below.

In some embodiments, nucleic acid barcode molecules can be initially associated with the bead and then released from the bead. In some embodiments, release of the nucleic acid barcode molecules can be passive (e.g., by diffusion out of the bead). In addition or alternatively, release from the bead can be upon application of a stimulus which allows the nucleic acid barcode molecules to dissociate or to be released from the bead. Such stimulus can disrupt the bead, an interaction that couples the barcoded nucleic acid molecules to or within the bead, or both. Such stimulus can include, for example, a thermal stimulus, photo-stimulus, chemical stimulus (e.g., change in pH or use of a reducing agent), a mechanical stimulus, a radiation stimulus; a biological stimulus (e.g., enzyme), or any combination thereof. Methods and systems for partitioning barcode carrying beads into droplets are provided in US. Patent Publication Nos. 2019/0367997 and 2019/0064173, and International Application Nos. PCT/US20/17785 and PCT/US20/020486.

Beneficially, a discrete droplet partitioning a biological particle and a barcode carrying bead can effectively allow the attribution of the barcode to macromolecular constituents of the biological particle within the partition. The contents of a partition can remain discrete from the contents of other partitions.

In operation, the barcoded oligonucleotides can be released (e.g., in a partition), as described elsewhere herein. Alternatively, the nucleic acid barcode molecules bound to the bead (e.g., gel bead) can be used to hybridize and capture analytes (e.g., one or more types of analytes) on the solid phase of the bead.

In some examples, beads, biological particles (e.g., labelled cells such as engineered cells, B cells, or nuclei) and droplets can flow along channels (e.g., the channels of a microfluidic device), in some cases at substantially regular flow profiles (e.g., at regular flow rates). Such regular flow profiles can permit a droplet to include a single bead and a single biological particle. Such regular flow profiles can permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that can be used to provide such regular flow profiles are provided in, for example, U.S. Patent Publication No. 2015/0292988.

A bead can be porous, non-porous, solid, semi-solid, semi-fluidic, fluidic, and/or a combination thereof. In some instances, a bead can be dissolvable, disruptable, and/or degradable. In some cases, a bead cannot be degradable. In some cases, the bead can be a gel bead. A gel bead can be a hydrogel bead. A gel bead can be formed from molecular precursors, such as a polymeric or monomeric species. A semi-solid bead can be a liposomal bead. Solid beads can include metals including iron oxide, gold, and silver. In some cases, the bead can be a silica bead. In some cases, the bead can be rigid. In other cases, the bead can be flexible and/or compressible.

A bead can be of any suitable shape. Examples of bead shapes include, but are not limited to, spherical, non-spherical, oval, oblong, amorphous, circular, cylindrical, and variations thereof.

Beads can be of uniform size or heterogeneous size. In some cases, the diameter of a bead can be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead can have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead can have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In some embodiments, the beads described herein can have size distributions that have a coefficient of variation in their cross-sectional dimensions of less than 50%, less than 40%, less than 30%, less than 20%, and in some cases less than 15%, less than 10%, less than 5%, or less.

A bead can include natural and/or synthetic materials. For example, a bead can include a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads can also be formed from materials other than polymers, including lipids, micelles, ceramics, glass-ceramics, material composites, metals, other inorganic materials, and others.

In some embodiments, the bead can contain molecular precursors (e.g., monomers or polymers), which can form a polymer network via polymerization of the molecular precursors. In some cases, a precursor can be an already polymerized species capable of undergoing further polymerization via, for example, a chemical cross-linkage. In some embodiments, a precursor can include one or more of an acrylamide or a methacrylamide monomer, oligomer, or polymer. In some cases, the bead can include prepolymers, which are oligomers capable of further polymerization. For example, polyurethane beads can be prepared using prepolymers. In some embodiments, the bead can contain individual polymers that can be further polymerized together. In some cases, beads can be generated via polymerization of different precursors, such that they include mixed polymers, co-polymers, and/or block co-polymers. In some embodiments, the bead can include covalent or ionic bonds between polymeric precursors (e.g., monomers, oligomers, linear polymers), nucleic acid molecules (e.g., nucleic acid barcode molecules or barcoded oligonucleotides), primers, and other entities. In some embodiments, the covalent bonds can be carbon-carbon bonds, thioether bonds, or carbon-heteroatom bonds.

Cross-linking can be permanent or reversible, depending upon the particular cross-linker used. Reversible cross-linking can allow for the polymer to linearize or dissociate under appropriate conditions. In some embodiments, reversible cross-linking can also allow for reversible attachment of a material bound to the surface of a bead. In some embodiments, a cross-linker can form disulfide linkages. In some embodiments, the chemical cross-linker forming disulfide linkages can be cystamine or a modified cystamine.

In some embodiments, disulfide linkages can be formed between molecular precursor units (e.g., monomers, oligomers, or linear polymers) or precursors incorporated into a bead and nucleic acid barcode molecules (e.g., oligonucleotides). Cystamine (including modified cystamines), for example, is an organic agent including a disulfide bond that can be used as a crosslinker agent between individual monomeric or polymeric precursors of a bead. Polyacrylamide can be polymerized in the presence of cystamine or a species including cystamine (e.g., a modified cystamine) to generate polyacrylamide gel beads including disulfide linkages (e.g., chemically degradable beads including chemically-reducible cross-linkers). The disulfide linkages can permit the bead to be degraded (or dissolved) upon exposure of the bead to a reducing agent.

In some embodiments, chitosan, a linear polysaccharide polymer, can be crosslinked with glutaraldehyde via hydrophilic chains to form a bead. Crosslinking of chitosan polymers can be achieved by chemical reactions that are initiated by heat, pressure, change in pH, and/or radiation.

In some embodiments, a bead can include an acrydite moiety, which in certain aspects can be used to attach one or more nucleic acid molecules (e.g., barcode sequence, nucleic acid barcode molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties can be modified to form chemical bonds with a species to be attached, such as a nucleic acid molecule (e.g., barcode sequence, nucleic acid barcode molecule, barcoded oligonucleotide, primer, or other oligonucleotide). Acrydite moieties can be modified with thiol groups capable of forming a disulfide bond or can be modified with groups already including a disulfide bond. The thiol or disulfide (via disulfide exchange) can be used as an anchor point for a species to be attached or another part of the acrydite moiety can be used for attachment. In some cases, attachment can be reversible, such that when the disulfide bond is broken (e.g., in the presence of a reducing agent), the attached species is released from the bead. In other cases, an acrydite moiety can include a reactive hydroxyl group that can be used for attachment.

Functionalization of beads for attachment of nucleic acid molecules (e.g., nucleic acid barcode molecules) can be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead can include acrydite moieties, such that when a bead is generated, the bead also includes acrydite moieties. The acrydite moieties can be attached to a nucleic acid molecule (e.g., nucleic acid barcode molecule), which can include a priming sequence (e.g., a primer for amplifying target nucleic acids, random primer, primer sequence for messenger RNA) and/or one or more barcode sequences. The one or more barcode sequences can include sequences that are the same for all nucleic acid barcode molecules coupled to a given bead and/or sequences that are different across all nucleic acid barcode molecules coupled to the given bead. The nucleic acid barcode molecule can be incorporated into the bead.

In some embodiments, the nucleic acid barcode molecule can include a functional sequence, for example, for attachment to a sequencing flow cell, such as, for example, a P5 sequence for Illumina® sequencing. In some cases, the nucleic acid barcode molecule or derivative thereof (e.g., oligonucleotide or polynucleotide generated from the nucleic acid barcode molecule) can include another functional sequence, such as, for example, a P7 sequence for attachment to a sequencing flow cell for Illumina sequencing. In some cases, the nucleic acid barcode molecule can include a barcode sequence. In some cases, the primer can further include a unique molecular identifier (UMI). In some cases, the primer can include an R1 primer sequence for Illumina sequencing. In some cases, the primer can include an R2 primer sequence for Illumina sequencing. Examples of such nucleic acid molecules (e.g., oligonucleotides, polynucleotides, etc.) and uses thereof, as can be used with compositions, devices, methods and systems of the present disclosure, are provided in U.S. Patent Pub. Nos. 2014/0378345 and 2015/0376609.

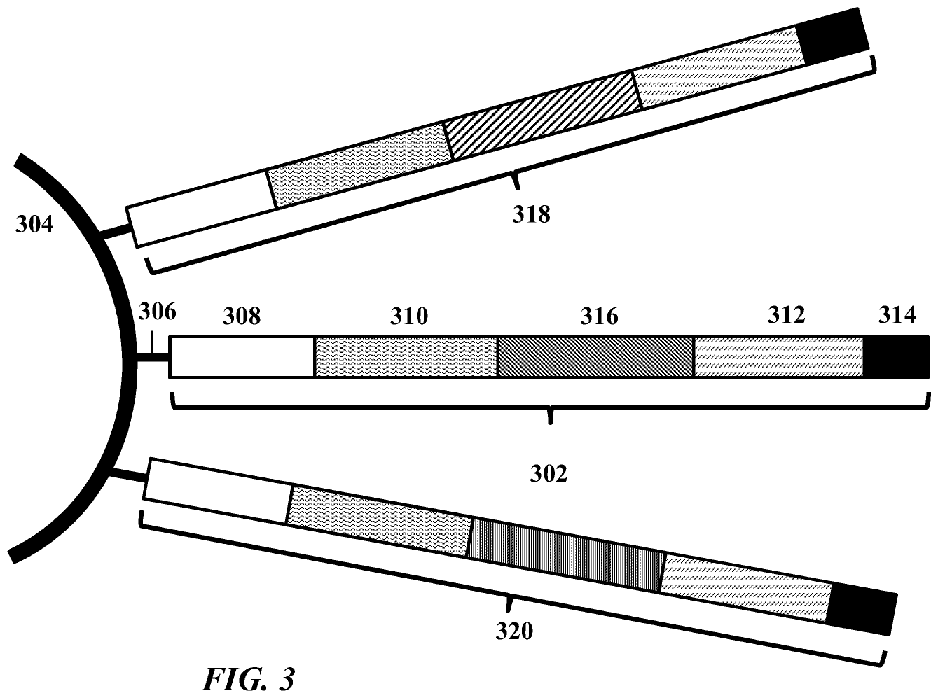

FIG. 3 illustrates an example of a barcode carrying bead. A nucleic acid molecule 302 can be coupled to a bead 304 by a releasable linkage 306, such as, for example, a disulfide linker. The same bead 304 can be coupled (e.g., via releasable linkage) to one or more other nucleic acid molecules 318, 320. The nucleic acid barcode molecule 302 can be or include a barcode. As noted elsewhere herein, the structure of the barcode can include a number of sequence elements. The nucleic acid barcode molecule 302 can include a functional sequence 308 that can be used in subsequent processing. For example, the functional sequence 308 can include one or more of a sequencer specific flow cell attachment sequence (e.g., a P5 sequence for Illumina® sequencing systems) and a sequencing primer sequence (e.g., a R1 primer for Illumina® sequencing systems). The nucleic acid barcode molecule 302 can include a barcode sequence 310 for use in barcoding the sample (e.g., DNA, RNA, protein, etc.). In some cases, the barcode sequence 310 can be bead-specific such that the barcode sequence 310 is common to all nucleic acid molecules (e.g., including nucleic acid barcode molecule 302) coupled to the same bead 304. Alternatively or in addition, the barcode sequence 310 can be partition-specific such that the barcode sequence 310 is common to all nucleic acid barcode molecules coupled to one or more beads that are partitioned into the same partition. The nucleic acid molecule 302 can include a specific priming sequence 312, such as an mRNA specific priming sequence (e.g., poly-T sequence), a targeted priming sequence, and/or a random priming sequence. The nucleic acid barcode molecule 302 can include an anchoring sequence 314 to ensure that the specific priming sequence 312 hybridizes at the sequence end (e.g., of the mRNA). For example, the anchoring sequence 314 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid barcode molecule 302 can include a unique molecular identifying sequence 316 (e.g., unique molecular identifier (UMI)). In some cases, the unique molecular identifying sequence 316 can include from about 5 to about 8 nucleotides. Alternatively, the unique molecular identifying sequence 316 can compress less than about 5 or more than about 8 nucleotides. The unique molecular identifying sequence 316 can be a unique sequence that varies across individual nucleic acid molecules (e.g., 302, 318, 320, etc.) coupled to a single bead (e.g., bead 304). In some cases, the unique molecular identifying sequence 316 can be a random sequence (e.g., such as a random N-mer sequence). For example, the UMI can provide a unique identifier of the starting mRNA molecule that was captured, in order to allow quantitation of the number of original expressed RNA. As will be appreciated, although FIG. 3 shows three nucleic acid barcode molecules 302, 318, 320 coupled to the surface of the bead 304, an individual bead can be coupled to any number of individual nucleic acid barcode molecules, for example, from one to tens to hundreds of thousands, millions, or even billion of individual nucleic acid barcode molecules. The respective barcodes for the individual nucleic acid molecules can include both common sequence segments or relatively common sequence segments (e.g., 308, 310, 312, etc.) and variable or unique sequence segments (e.g., 316) between different individual nucleic acid molecules coupled to the same bead.

In operation, a biological particle (e.g., cell, cell bead, nucleus, DNA, RNA, etc.) can be co-partitioned along with a barcode bearing bead 304. The nucleic acid barcode molecules 302, 318, 320 can be released from the bead 304 in the partition. By way of example, in the context of analyzing sample RNA, the poly-T segment (e.g., 312) of one of the released nucleic acid molecules (e.g., 302) can hybridize to the poly-A tail of a mRNA molecule. Reverse transcription can result in a cDNA transcript of the mRNA, but which transcript includes each of the sequence segments 308, 310, 316 of the nucleic acid molecule 302. Because the nucleic acid barcode molecule 302 includes an anchoring sequence 314, it will more likely hybridize to and prime reverse transcription at the sequence end of the poly-A tail of the mRNA. Within any given partition, all of the cDNA transcripts of the individual mRNA molecules can include a common barcode sequence segment 310. However, the transcripts made from the different mRNA molecules within a given partition can vary at the unique molecular identifying sequence 312 segment (e.g., UMI segment). Beneficially, even following any subsequent amplification of the contents of a given partition, the number of different UMIs can be indicative of the quantity of mRNA originating from a given partition, and thus from the biological particle (e.g., cell, cell bead, or nucleus). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences can also be used in priming the reverse transcription reaction. Likewise, although described as releasing the barcoded oligonucleotides into the partition, in some cases, the nucleic acid barcode molecules bound to the bead (e.g., gel bead) can be used to hybridize and capture the mRNA on the solid phase of the bead, for example, in order to facilitate the separation of the RNA from other cell or nuclear contents. In such cases, further processing can be performed, in the partitions or outside the partitions (e.g., in bulk). For instance, the RNA molecules on the beads can be subjected to reverse transcription or other nucleic acid processing, additional adapter sequences can be added to the barcoded nucleic acid molecules, or other nucleic acid reactions (e.g., amplification, nucleic acid extension) can be performed. The beads or products thereof (e.g., barcoded nucleic acid molecules) can be collected from the partitions, and/or pooled together and subsequently subjected to clean up and further characterization (e.g., sequencing).

The operations described herein can be performed at any useful or suitable step. For instance, the beads including nucleic acid barcode molecules can be introduced into a partition (e.g., well or droplet) prior to, during, or following introduction of a sample into the partition. The nucleic acid molecules of a sample can be subjected to barcoding, which can occur on the bead (in cases where the nucleic acid molecules remain coupled to the bead) or following release of the nucleic acid barcode molecules into the partition. In cases where analytes from the sample are captured by the nucleic acid barcode molecules in a partition (e.g., by hybridization), captured analytes from various partitions may be collected, pooled, and subjected to further processing (e.g., reverse transcription, adapter attachment, amplification, clean up, sequencing). For example, in cases wherein the nucleic acid molecules from the sample remain attached to the bead, the beads from various partitions can be collected, pooled, and subjected to further processing (e.g., reverse transcription, adapter attachment, amplification, clean up, and/or sequencing). In other instances, one or more of the processing methods, e.g., reverse transcription, can occur in the partition. For example, conditions sufficient for barcoding, adapter attachment, reverse transcription, or other nucleic acid processing operations can be provided in the partition and performed prior to clean up and sequencing.

In some instances, a bead can include a capture sequence or binding sequence configured to bind to a corresponding capture sequence or binding sequence. In some instances, a bead can include a plurality of different capture sequences or binding sequences configured to bind to different respective corresponding capture sequences or binding sequences. For example, a bead can include a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead can include any number of different capture sequences. In some instances, a bead can include at least 2, 3, 4, 5, 6, 7, 8, 9, 10 or more different capture sequences or binding sequences configured to bind to different respective capture sequences or binding sequences, respectively. Alternatively or in addition, a bead can include at most about 10, 9, 8, 7, 6, 5, 4, 3, or 2 different capture sequences or binding sequences configured to bind to different respective capture sequences or binding sequences. In some instances, the different capture sequences or binding sequences can be configured to facilitate analysis of a same type of analyte. In some instances, the different capture sequences or binding sequences can be configured to facilitate analysis of different types of analytes (with the same bead). The capture sequence can be designed to attach to a corresponding capture sequence. Beneficially, such corresponding capture sequence can be introduced to, or otherwise induced in, a biological particle (e.g., cell, cell bead, etc.) for performing different assays in various formats (e.g., barcoded antibodies including the corresponding capture sequence, barcoded MHC dextramers including the corresponding capture sequence, barcoded guide RNA molecules including the corresponding capture sequence, etc.), such that the corresponding capture sequence can later interact with the capture sequence associated with the bead. In some instances, a capture sequence coupled to a bead (or other support) can be configured to attach to a linker molecule, such as a splint molecule, wherein the linker molecule is configured to couple the bead (or other support) to other molecules through the linker molecule, such as to one or more analytes or one or more other linker molecules.

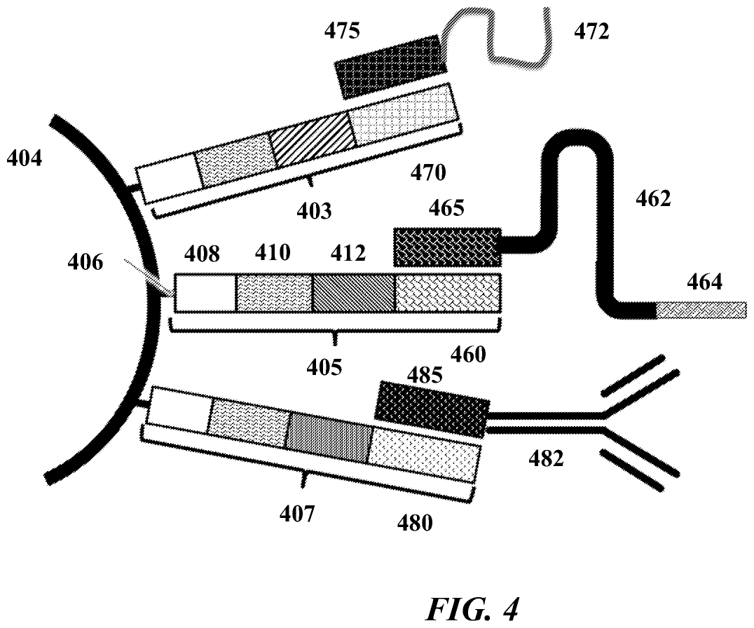

FIG. 4 illustrates a non-limiting example of a barcode carrying bead in accordance with some embodiments of the disclosure. A nucleic acid barcode molecule 405, such as an oligonucleotide, can be coupled to a bead 404 by a releasable linkage 406, such as, for example, a disulfide linker. The nucleic acid barcode molecule 405 can include a first capture sequence 460. The same bead 404 can be coupled, e.g., via releasable linkage, to one or more other nucleic acid molecules 403, 407 including other capture sequences. The nucleic acid barcode molecule 405 can be or include a barcode sequence. As described elsewhere herein, the structure of the barcode sequence can include a number of sequence elements, such as a functional sequence 408 (e.g., flow cell attachment sequence, sequencing primer sequence, etc.), a barcode sequence 410 (e.g., bead-specific sequence common to bead, partition-specific sequence common to partition, etc.), and a unique molecular identifier 412 (e.g., unique sequence within different molecules attached to the bead), or partial sequences thereof. The capture sequence 460 can be configured to attach to a corresponding capture sequence 465 (e.g., capture handle). In some instances, the corresponding capture sequence 465 can be coupled to another molecule that can be an analyte or an intermediary carrier. For example, as illustrated in FIG. 4, the corresponding capture sequence 465 is coupled to a guide RNA molecule 462 including a target sequence 464, wherein the target sequence 464 is configured to attach to the analyte. Another oligonucleotide molecule 407 attached to the bead 404 includes a second capture sequence 480 which is configured to attach to a second corresponding capture sequence (e.g., capture handle) 485. As illustrated in FIG. 4, the second corresponding capture sequence 485 is coupled to an antibody 482. In some cases, the antibody 482 can have binding specificity to an analyte (e.g., surface protein). Alternatively, the antibody 482 cannot have binding specificity. Another oligonucleotide molecule 403 attached to the bead 404 includes a third capture sequence 470 which is configured to attach to a second corresponding capture sequence 475. As illustrated in FIG. 4, the third corresponding capture sequence (e.g., capture handle) 475 is coupled to a molecule 472. The molecule 472 may or may not be configured to target an analyte. The other oligonucleotide molecules 403, 407 can include the other sequences (e.g., functional sequence, barcode sequence, UMI, etc.) described with respect to oligonucleotide molecule 405. While a single oligonucleotide molecule including each capture sequence is illustrated in FIG. 4, it will be appreciated that, for each capture sequence, the bead can include a set of one or more oligonucleotide molecules each including the capture sequence. For example, the bead can include any number of sets of one or more different capture sequences. Alternatively or in addition, the bead 404 can include other capture sequences. Alternatively or in addition, the bead 404 can include fewer types of capture sequences (e.g., two capture sequences). Alternatively or in addition, the bead 404 can include oligonucleotide molecule(s) including a priming sequence, such as a specific priming sequence such as an mRNA specific priming sequence (e.g., poly-T sequence), a targeted priming sequence, and/or a random priming sequence, for example, to facilitate an assay for gene expression.

The generation of a barcoded sequence, see, e.g., FIG. 3, is described herein.

In some embodiments, precursors including a functional group that is reactive or capable of being activated such that it becomes reactive can be polymerized with other precursors to generate gel beads including the activated or activatable functional group. The functional group can then be used to attach additional species (e.g., disulfide linkers, primers, other oligonucleotides, etc.) to the gel beads. For example, some precursors including a carboxylic acid (COOH) group can co-polymerize with other precursors to form a gel bead that also includes a COOH functional group. In some cases, acrylic acid (a species including free COOH groups), acrylamide, and bis(acryloyl)cystamine can be co-polymerized together to generate a gel bead including free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species including an amine functional group where the carboxylic acid groups are activated to be reactive with an amine functional group) including a moiety to be linked to the bead.

Beads including disulfide linkages in their polymeric network can be functionalized with additional species via reduction of some of the disulfide linkages to free thiols. The disulfide linkages can be reduced via, for example, the action of a reducing agent (e.g., DTT, TCEP, etc.) to generate free thiol groups, without dissolution of the bead. Free thiols of the beads can then react with free thiols of a species or a species including another disulfide bond (e.g., via thiol-disulfide exchange) such that the species can be linked to the beads (e.g., via a generated disulfide bond). In some cases, free thiols of the beads can react with any other suitable group. For example, free thiols of the beads can react with species including an acrydite moiety. The free thiol groups of the beads can react with the acrydite via Michael addition chemistry, such that the species including the acrydite is linked to the bead. In some cases, uncontrolled reactions can be prevented by inclusion of a thiol capping agent such as N-ethylmaleiamide or iodoacetate.

Activation of disulfide linkages within a bead can be controlled such that only a small number of disulfide linkages are activated. Control can be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent can be used for reduction. Controlling the number of disulfide linkages that are reduced to free thiols can be useful in ensuring bead structural integrity during functionalization. In some cases, optically-active agents, such as fluorescent dyes can be coupled to beads via free thiol groups of the beads and used to quantify the number of free thiols present in a bead and/or track a bead.

In some embodiments, addition of moieties to a gel bead after gel bead formation can be advantageous. For example, addition of an oligonucleotide (e.g., barcoded oligonucleotide, such as a nucleic acid barcode molecule) after gel bead formation can avoid loss of the species during chain transfer termination that can occur during polymerization. Moreover, smaller precursors (e.g., monomers or cross linkers that do not include side chain groups and linked moieties) can be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel can possess an upper critical solution temperature (UCST) that can permit temperature driven swelling and collapse of a bead. Such functionality can aid in oligonucleotide (e.g., a primer) infiltration into the bead during subsequent functionalization of the bead with the oligonucleotide. Post-production functionalization can also be useful in controlling loading ratios of species in beads, such that, for example, the variability in loading ratio is minimized. Species loading can also be performed in a batch process such that a plurality of beads can be functionalized with the species in a single batch.

A bead injected or otherwise introduced into a partition can include releasably, cleavably, or reversibly attached barcodes (e.g., partition barcode sequences). A bead injected or otherwise introduced into a partition can include activatable barcodes. A bead injected or otherwise introduced into a partition can be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage can be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as nucleic acid barcode molecules (e.g., barcoded oligonucleotides), the beads can be degradable, disruptable, or dissolvable spontaneously or upon exposure to one or more stimuli (e.g., temperature changes, pH changes, exposure to particular chemical species or phase, exposure to light, reducing agent, etc.). In some cases, a bead can be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead can be thermally degradable such that when the bead is exposed to an appropriate change in temperature (e.g., heat), the bead degrades. Degradation or dissolution of a bead bound to a species (e.g., a nucleic acid molecule, e.g., nucleic acid barcode molecule or barcoded oligonucleotide) can result in release of the species from the bead.

As will be appreciated from the above disclosure, the degradation of a bead can refer to the disassociation of a bound (e.g., capture agent configured to couple to a secreted antibody or antigen-binding fragment thereof) or entrained species (e.g., labelled cells such as labelled engineered cells, B cells, e.g., memory B cells, or plasma cells, or secreted antibody or antigen-binding fragment thereof) from a bead, both with and without structurally degrading the physical bead itself. For example, the degradation of the bead can involve cleavage of a cleavable linkage via one or more species and/or methods described elsewhere herein. In another example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

A degradable bead can be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid barcode molecules) can interact with other reagents contained in the partition. For example, a polyacrylamide bead including cystamine and linked, via a disulfide bond, to a barcode sequence, can be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet including a bead-bound barcode sequence in basic solution can also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing nucleic acid barcode molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads can be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads can be accomplished by various swelling methods. The de-swelling of the beads can be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads can be accomplished by various de-swelling methods. Transferring the beads can cause pores in the bead to shrink. The shrinking can then hinder reagents within the beads from diffusing out of the interiors of the beads. The hindrance can be due to steric interactions between the reagents and the interiors of the beads. The transfer can be accomplished microfluidically. For instance, the transfer can be achieved by moving the beads from one co-flowing solvent stream to a different co-flowing solvent stream. The swellability and/or pore size of the beads can be adjusted by changing the polymer composition of the bead.

In some cases, an acrydite moiety linked to a precursor, another species linked to a precursor, or a precursor itself can include a labile bond, such as chemically, thermally, or photo-sensitive bond e.g., disulfide bond, UV sensitive bond, or the like. Once acrydite moieties or other moieties including a labile bond are incorporated into a bead, the bead can also include the labile bond. The labile bond can be, for example, useful in reversibly linking (e.g., covalently linking) species (e.g., barcodes, primers, etc.) to a bead. In some cases, a thermally labile bond can include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead.

The addition of multiple types of labile bonds to a gel bead can result in the generation of a bead capable of responding to varied stimuli. Each type of labile bond can be sensitive to an associated stimulus (e.g., chemical stimulus, light, temperature, enzymatic, etc.) such that release of species attached to a bead via each labile bond can be controlled by the application of the appropriate stimulus. Such functionality can be useful in controlled release of species from a gel bead. In some cases, another species including a labile bond can be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that can be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond can be cleavable via other nucleic acid molecule targeting enzymes, such as restriction enzymes (e.g., restriction endonucleases), as described further below.

Species can be encapsulated in beads (e.g., capture agent) during bead generation (e.g., during polymerization of precursors). Such species may or may not participate in polymerization. Such species can be entered into polymerization reaction mixtures such that generated beads include the species upon bead formation. In some cases, such species can be added to the gel beads after formation. Such species can include, for example, nucleic acid molecules (e.g., oligonucleotides, e.g., nucleic acid barcode molecules), reagents for a nucleic acid amplification reaction (e.g., primers, polymerases, dNTPs, co-factors (e.g., ionic co-factors, buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species can include one or more enzymes described herein, including without limitation, polymerase, reverse transcriptase, restriction enzymes (e.g., endonuclease), transposase, ligase, proteinase K, DNAse, etc. Such species can include one or more reagents described elsewhere herein (e.g., lysis agents, inhibitors, inactivating agents, chelating agents, stimulus). Trapping of such species can be controlled by the polymer network density generated during polymerization of precursors, control of ionic charge within the gel bead (e.g., via ionic species linked to polymerized species), or by the release of other species. Encapsulated species can be released from a bead upon bead degradation and/or by application of a stimulus capable of releasing the species from the bead. Alternatively or in addition, species can be partitioned in a partition (e.g., droplet) during or subsequent to partition formation. Such species can include, without limitation, the abovementioned species that can also be encapsulated in a bead.

A degradable bead can include one or more species with a labile bond such that, when the bead/species is exposed to the appropriate stimuli, the bond is broken and the bead degrades. The labile bond can be a chemical bond (e.g., covalent bond, ionic bond) or can be another type of physical interaction (e.g., van der Waals interactions, dipole-dipole interactions, etc.). In some cases, a crosslinker used to generate a bead can include a labile bond. Upon exposure to the appropriate conditions, the labile bond can be broken and the bead degraded. For example, upon exposure of a polyacrylamide gel bead including cystamine crosslinkers to a reducing agent, the disulfide bonds of the cystamine can be broken and the bead degraded.

A degradable bead can be useful in more quickly releasing an attached species (e.g., a nucleic acid molecule, nucleic acid barcode molecule, a barcode sequence, a primer, etc.) from the bead when the appropriate stimulus is applied to the bead as compared to a bead that does not degrade. For example, for a species bound to an inner surface of a porous bead or in the case of an encapsulated species, the species can have greater mobility and accessibility to other species in solution upon degradation of the bead. In some cases, a species can also be attached to a degradable bead via a degradable linker (e.g., disulfide linker). The degradable linker can respond to the same stimuli as the degradable bead or the two degradable species can respond to different stimuli. For example, a barcode sequence can be attached, via a disulfide bond, to a polyacrylamide bead including cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation can refer to the disassociation of a bound or entrained species from a bead, both with and without structurally degrading the physical bead itself. For example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

Where degradable beads are provided, it can be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads include reducible cross-linking groups, such as disulfide groups, it will be desirable to avoid contacting such beads with reducing agents, e.g., DT or other disulfide cleaving reagents. In such cases, treatment to the beads described herein will, in some cases be provided free of reducing agents, such as DT. Because reducing agents are often provided in commercial enzyme preparations, it can be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that can be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers can be used to trigger the degradation of beads. Examples of these chemical changes can include, but are not limited to pH-mediated changes to the integrity of a component within the bead, degradation of a component of a bead via cleavage of cross-linked bonds, and depolymerization of a component of a bead.

In some embodiments, a bead can be formed from materials that include degradable chemical crosslinkers, such as BAC or cystamine. Degradation of such degradable crosslinkers can be accomplished through a number of mechanisms. In some examples, a bead can be contacted with a chemical degrading agent that can induce oxidation, reduction or other chemical changes. For example, a chemical degrading agent can be a reducing agent, such as dithiothreitol (DTT). Additional examples of reducing agents can include β-mercaptoethanol, (2S)-2-amino-1,4-dimercaptobutane (dithiobutylamine or DTBA), tris(2-carboxyethyl) phosphine (TCEP), or combinations thereof. A reducing agent can degrade the disulfide bonds formed between gel precursors forming the bead, and thus, degrade the bead. In other cases, a change in pH of a solution, such as an increase in pH, can trigger degradation of a bead. In other cases, exposure to an aqueous solution, such as water, can trigger hydrolytic degradation, and thus degradation of the bead. In some cases, any combination of stimuli can trigger degradation of a bead. For example, a change in pH can enable a chemical agent (e.g., DT) to become an effective reducing agent.

Beads can also be induced to release their contents upon the application of a thermal stimulus. A change in temperature can cause a variety of changes to a bead. For example, heat can cause a solid bead to liquefy. A change in heat can cause melting of a bead such that a portion of the bead degrades. In other cases, heat can increase the internal pressure of the bead components such that the bead ruptures or explodes. Heat can also act upon heat-sensitive polymers used as materials to construct beads.

Any suitable agent can degrade beads. In some embodiments, changes in temperature or pH can be used to degrade thermo-sensitive or pH-sensitive bonds within beads. In some embodiments, chemical degrading agents can be used to degrade chemical bonds within beads by oxidation, reduction or other chemical changes. For example, a chemical degrading agent can be a reducing agent, such as DTT, wherein DTT can degrade the disulfide bonds formed between a crosslinker and gel precursors, thus degrading the bead. In some embodiments, a reducing agent can be added to degrade the bead, which may or may not cause the bead to release its contents. Examples of reducing agents can include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent can be present at a concentration of about 0.1 mM, 0.5 mM, 1 mM, 5 mM, or 10 mM. The reducing agent can be present at a concentration of at least about 0.1 mM, 0.5 mM, 1 mM, 5 mM, 10 mM, or greater than 10 mM. The reducing agent can be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more biological particles (e.g., cells, cell beads, or nuclei) and/or beads including nucleic acid barcode molecules (e.g., oligonucleotides) within a single partition (e.g., multi-omics method described elsewhere, herein). Accordingly, as noted above, the flow characteristics of the biological particle and/or bead containing fluids and partitioning fluids can be controlled to provide for such multiply occupied partitions. In particular, the flow parameters can be controlled to provide a given occupancy rate at greater than about 50% of the partitions, greater than about 75%, and in some cases greater than about 80%, 90%, 95%, or higher.

In some cases, additional beads can be used to deliver additional reagents to a partition. In such cases, it can be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction can be controlled to provide for a certain ratio of beads from each source, while ensuring a given pairing or combination of such beads into a partition with a given number of biological particles (e.g., one biological particle and one bead per partition).

The partitions described herein can include small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets can have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with beads, it will be appreciated that the sample fluid volume, e.g., including co-partitioned biological particles and/or beads, within the partitions can be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species can generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions can include both unoccupied partitions (e.g., empty partitions) and occupied partitions.

### Reagents

In accordance with certain aspects, biological particles can be partitioned along with lysis reagents in order to release the contents of the biological particles within the partition. See, e.g., U.S. Pat. Pub. 2018/0216162 (now U.S. Pat. No. 10,428,326), U.S. Pat. Pub. 2019/0100632 (now U.S. Pat. No. 10,590,244), and U.S. Pat. Pub. 2019/0233878. Biological particles (e.g., cells, cell beads, cell nuclei, organelles, and the like) can be partitioned together with nucleic acid barcode molecules and the nucleic acid molecules of or derived from the biological particle (e.g., mRNA, cDNA, gDNA, etc.,) can be barcoded as described elsewhere herein. In some embodiments, biological particles are co-partitioned with barcode carrying beads (e.g., gel beads) and the nucleic acid molecules of or derived from the biological particle are barcoded as described elsewhere herein. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles can be partitioned along with other reagents, as will be described further below.

Beneficially, when lysis reagents and biological particles are co-partitioned, the lysis reagents can facilitate the release of the contents of the biological particles within the partition. The contents released in a partition can remain discrete from the contents of other partitions.

As will be appreciated, the channel segments described herein can be coupled to any of a variety of different fluid sources or receiving components, including reservoirs, tubing, manifolds, or fluidic components of other systems. As will be appreciated, the microfluidic channel structures can have other geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment can be controlled to control the partitioning of the different elements into droplets. Fluid can be directed flow along one or more channels or reservoirs via one or more fluid flow units. A fluid flow unit can include compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents can additionally or alternatively be co-partitioned with the biological particles to cause the release of the biological particle's contents into the partitions. For example, in some cases, surfactant-based lysis solutions can be used to lyse cells (e.g., labelled cells such as labelled engineered cells or B cells, e.g., memory B cells), although these can be less desirable for emulsion based systems where the surfactants can interfere with stable emulsions. In some cases, lysis solutions can include non-ionic surfactants such as, for example, Triton X-100 and Tween 20. In some cases, lysis solutions can include ionic surfactants such as, for example, sarcosyl and sodium dodecyl sulfate (SDS). Electroporation, thermal, acoustic or mechanical cellular disruption can also be used in certain cases, e.g., non-emulsion based partitioning such as encapsulation of biological particles that can be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles (e.g., labelled cells such as labelled engineered cells or B cells, e.g., memory B cells) described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., cell beads comprising labelled cells such as labelled engineered cells, B cells, e.g., memory B cells), the biological particles can be exposed to an appropriate stimulus to release the biological particles or their contents from a co-partitioned cell bead. For example, in some cases, a chemical stimulus can be co-partitioned along with an encapsulated biological particle to allow for the degradation of the encapsulating material and release of the cell or its contents into the larger partition. In some cases, this stimulus can be the same as the stimulus described elsewhere herein for release of nucleic acid molecules (e.g., nucleic acid barcode molecules or barcoded oligonucleotides) from their respective bead. In alternative aspects, this can be a different and non-overlapping stimulus, in order to allow an encapsulated biological particle to be released into a partition at a different time from the release of nucleic acid molecules (e.g., nucleic acid barcode molecules or barcoded oligonucleotides) into the same partition.

Additional reagents can also be co-partitioned with the biological particles (e.g., labelled cells such as labelled engineered cells, B cells, or nuclei), such as endonucleases to fragment a biological particle's DNA, DNA polymerase enzymes and dNTPs used to amplify the biological particle's nucleic acid fragments and to attach the barcode molecular tags to the amplified fragments. Other enzymes can be co-partitioned, including without limitation, polymerase, transposase, ligase, proteinase K, DNAse, etc. Additional reagents can also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides can include a hybridization region and a template region. The hybridization region can include any sequence capable of hybridizing to the target. In some cases, as previously described, the hybridization region includes a series of G bases to complement the overhanging C bases at the 3′ end of a cDNA molecule. The series of G bases can include 1 G base, 2 G bases, 3 G bases, 4 G bases, 5 G bases or more than 5 G bases. The template sequence can include any sequence to be incorporated into the cDNA. In some cases, the template region includes at least 1 (e.g., at least 2, 3, 4, 5 or more) tag sequences and/or functional sequences. Switch oligos can include deoxyribonucleic acids; ribonucleic acids; modified nucleic acids including 2-Aminopurine, 2,6-Diaminopurine (2-Amino-dA), inverted dT, 5-Methyl dC, 2′-deoxylnosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo can be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells (e.g., labelled cells such as labelled engineered cells or B cells, e.g., memory B cells) are released into their respective partitions, the macromolecular components (e.g., macromolecular constituents of biological particles, such as RNA, DNA, proteins, or secreted antibodies or antigen-binding fragments thereof) contained therein can be further processed within the partitions. In accordance with the methods and systems described herein, the macromolecular component contents of individual biological particles (e.g., labelled cells such as labelled engineered cells or B cells, e.g., memory B cells) can be provided with unique identifiers such that, upon characterization of those macromolecular components they can be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle (e.g., labelled cell such as engineered cell, B cell, cell bead, nucleus) or groups of biological particles (e.g., labelled cells, labelled engineered cells, B cells, cell beads, nuclei) with the unique identifiers, such as described above (with reference to FIGS. 1 and 2). In some aspects, the unique identifiers are provided in the form of nucleic acid barcode molecules (e.g., oligonucleotides) that include nucleic acid barcode sequences that can be attached to or otherwise associated with the nucleic acid contents of individual biological particle, or to other components of the biological particle, and particularly to fragments of those nucleic acids. The nucleic acid barcode molecules are partitioned such that as between nucleic acid barcode molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences can be present.

The nucleic acid barcode sequences can include from about 6 to about 20 or more nucleotides within the sequence of the nucleic acid barcode molecules (e.g., oligonucleotides). The nucleic acid barcode sequences can include from about 6 to about 20, 30, 40, 50, 60, 70, 80, 90, 100 or more nucleotides. In some cases, the length of a barcode sequence can be about 6, 7, 8, 9, 10, 11, 12, 13, 14, 15, 16, 17, 18, 19, 20 nucleotides or longer. In some cases, the length of a barcode sequence can be at least about 6, 7, 8, 9, 10, 11, 12, 13, 14, 15, 16, 17, 18, 19, 20 nucleotides or longer. In some cases, the length of a barcode sequence can be at most about 6, 7, 8, 9, 10, 11, 12, 13, 14, 15, 16, 17, 18, 19, 20 nucleotides or shorter. These nucleotides can be completely contiguous, i.e., in a single stretch of adjacent nucleotides, or they can be separated into two or more separate subsequences that are separated by 1 or more nucleotides. In some cases, separated barcode subsequences can be from about 4 to about 16 nucleotides in length. In some cases, the barcode subsequence can be about 4, 5, 6, 7, 8, 9, 10, 11, 12, 13, 14, 15, 16 nucleotides or longer. In some cases, the barcode subsequence can be at least about 4, 5, 6, 7, 8, 9, 10, 11, 12, 13, 14, 15, 16 nucleotides or longer. In some cases, the barcode subsequence can be at most about 4, 5, 6, 7, 8, 9, 10, 11, 12, 13, 14, 15, 16 nucleotides or shorter.

The co-partitioned nucleic acid barcode molecules can also include other functional sequences useful in the processing of the nucleic acids from the co-partitioned biological particles (e.g., labelled cells such as labelled engineered cells B cells, e.g., memory B cells, or nuclei). These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides can also be employed, including, e.g., coalescence of two or more droplets, where one droplet contains oligonucleotides, or microdispensing of oligonucleotides into partitions, e.g., droplets within microfluidic systems.

In an example, beads are provided that each include large numbers of the above described nucleic acid barcode molecules (e.g., barcoded oligonucleotides) releasably attached to the beads, where all of the nucleic acid barcode molecules attached to a particular bead will include the same nucleic acid barcode sequence, but where a large number of diverse barcode sequences are represented across the population of beads used. In some embodiments, hydrogel beads, e.g., including polyacrylamide polymer matrices, are used as a solid support and delivery vehicle for the nucleic acid barcode molecules into the partitions, as they are capable of carrying large numbers of nucleic acid barcode molecules, and can be configured to release those nucleic acid barcode molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) barcode molecules attached. In particular, the number of nucleic acid barcode molecules including the barcode sequence on an individual bead can be at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acid barcode molecules, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules, or more. Nucleic acid barcode molecules of a given bead can include identical (or common) barcode sequences, different barcode sequences, or a combination of both. Nucleic acid barcode molecules of a given bead can include multiple sets of nucleic acid barcode molecules. Nucleic acid barcode molecules of a given set can include identical barcode sequences. The identical barcode sequences can be different from barcode sequences of nucleic acid barcode molecules of another set. In some embodiments, such different barcode sequences can be associated with a given bead.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acid barcode molecules, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules.

In some cases, it may be desirable to incorporate multiple different barcodes within a given partition, either attached to a single or multiple beads within the partition. For example, in some cases, a mixed, but known set of barcode sequences can provide greater assurance of identification in the subsequent processing, e.g., by providing a stronger address or attribution of the barcodes to a given partition, as a duplicate or independent confirmation of the output from a given partition.

The nucleic acid barcode molecules (e.g., oligonucleotides) are releasable from the beads upon the application of a particular stimulus to the beads. In some cases, the stimulus can be a photo-stimulus, e.g., through cleavage of a photo-labile linkage that releases the nucleic acid barcode molecules. In other cases, a thermal stimulus can be used, where elevation of the temperature of the beads environment will result in cleavage of a linkage or other release of the nucleic acid molecules from the beads. In still other cases, a chemical stimulus can be used that cleaves a linkage of the nucleic acid barcode molecules to the beads, or otherwise results in release of the nucleic acid barcode molecules from the beads. In one case, such compositions include the polyacrylamide matrices described above for encapsulation of biological particles, and can be degraded for release of the attached nucleic acid barcode molecules through exposure to a reducing agent, such as DTT.

### Systems and Methods for Controlled Partitioning

In some aspects, provided are systems and methods for controlled partitioning. Droplet size can be controlled by adjusting certain geometric features in channel architecture (e.g., microfluidics channel architecture). For example, an expansion angle, width, and/or length of a channel can be adjusted to control droplet size.

FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 can be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, h, a, etc.) of the channel structure 200. A plurality of droplets can be collected in the reservoir 204 by continuously injecting the aqueous fluid 208 from the channel segment 202 through the junction 206.

A discrete droplet generated can include a bead (e.g., as in occupied droplets 216). Alternatively, a discrete droplet generated can include more than one bead. Alternatively, a discrete droplet generated cannot include any beads (e.g., as in unoccupied droplet 218). In some instances, a discrete droplet generated can contain one or more biological particles, as described elsewhere herein. In some instances, a discrete droplet generated can include one or more reagents, as described elsewhere herein.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 can be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 208 in the channel segment 202 can include biological particles (e.g., described with reference to FIG. 1). In some instances, the aqueous fluid 208 can have a substantially uniform concentration or frequency of biological particles (e.g., cells or nuclei). As with the beads, the biological particles (e.g., labelled cells such as labelled engineered cells, B cells, or nuclei) can be introduced into the channel segment 202 from a separate channel. The frequency or concentration of the biological particles in the aqueous fluid 208 in the channel segment 202 can be controlled by controlling the frequency in which the biological particles are introduced into the channel segment 202 and/or the relative flow rates of the fluids in the channel segment 202 and the separate channel. In some instances, the biological particles (e.g., cells or nuclei) can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads can be upstream or downstream of the second separate channel introducing the biological particles.

The second fluid 210 can include an oil, such as a fluorinated oil, that includes a fluorosurfactant for stabilizing the resulting droplets, for example, inhibiting subsequent coalescence of the resulting droplets.

In some instances, the second fluid 210 cannot be subjected to and/or directed to any flow in or out of the reservoir 204. For example, the second fluid 210 can be substantially stationary in the reservoir 204. In some instances, the second fluid 210 can be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 can be subjected and/or directed to flow in or out of the reservoir 204. For example, the reservoir 204 can be a channel directing the second fluid 210 from upstream to downstream, transporting the generated droplets.

The channel structure 200 at or near the junction 206 can have certain geometric features that at least partly determine the sizes of the droplets formed by the channel structure 200. The channel segment 202 can have a height, h₀ and width, w, at or near the junction 206. By way of example, the channel segment 202 can include a rectangular cross-section that leads to a reservoir 204 having a wider cross-section (such as in width or diameter). Alternatively, the cross-section of the channel segment 202 can be other shapes, such as a circular shape, trapezoidal shape, polygonal shape, or any other shapes. The top and bottom walls of the reservoir 204 at or near the junction 206 can be inclined at an expansion angle, a. The expansion angle, a, allows the tongue (portion of the aqueous fluid 208 leaving channel segment 202 at junction 206 and entering the reservoir 204 before droplet formation) to increase in depth and facilitate decrease in curvature of the intermediately formed droplet. Droplet size can decrease with increasing expansion angle. The resulting droplet radius, R_{d}, can be predicted by the following equation for the aforementioned geometric parameters of h₀, w, and α:

$${R}_{d} \approx 0.44 ( 1 + 2.2 \sqrt{tan a} \frac{w}{{h}_{0}} ) \frac{{h}_{0}}{\sqrt{tan a}}$$

By way of example, for a channel structure with w=21 μm, h=21 μm, and α=3°, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7°, the predicted droplet size is 124 μm.

In some instances, the expansion angle, a, can be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 208 entering the junction 206 can be between about 0.04 microliters (μL)/minute (min) and about 40 μL/min. In some instances, the flow rate of the aqueous fluid 208 entering the junction 206 can be between about 0.01 microliters (μL)/minute (min) and about 100 μL/min. Alternatively, the flow rate of the aqueous fluid 208 entering the junction 206 can be less than about 0.01 μL/min. Alternatively, the flow rate of the aqueous fluid 208 entering the junction 206 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius cannot be dependent on the flow rate of the aqueous fluid 208 entering the junction 206.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 206) between aqueous fluid 208 channel segments (e.g., channel segment 202) and the reservoir 204. Alternatively or in addition, the throughput of droplet generation can be increased by increasing the flow rate of the aqueous fluid 208 in the channel segment 202.

The methods and systems described herein can be used to greatly increase the efficiency of single cell applications and/or other applications receiving droplet-based input.

Subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations can occur in bulk (e.g., outside the partition). In the case where a partition is a droplet in an emulsion, the emulsion can be broken and the contents of the droplet pooled for additional operations. Additional reagents that can be co-partitioned along with the barcode bearing bead can include oligonucleotides to block ribosomal RNA (rRNA) and nucleases to digest genomic DNA from cells. Alternatively, rRNA removal agents can be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, can be subject to sequencing for sequence analysis. In some cases, amplification can be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

Partitions including a barcode bead (e.g., a gel bead) associated with barcode molecules and a bead encapsulating cellular constituents (e.g., a cell bead) such as cellular nucleic acids can be useful in constituent analysis as is described in U.S. Patent Publication No. 2018/0216162.

### Sample and Cell Processing

A sample can be derived from any useful source including any subject, such as a human subject. A sample can include material (e.g., one or more cells) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, can be obtained for analysis as described herein. For example, a first sample can be obtained from a subject at a first time and a second sample can be obtained from the subject at a second time later than the first time. The first time can be before a subject undergoes a treatment regimen or procedure (e.g., to address a disease or condition), and the second time can be during or after the subject undergoes the treatment regimen or procedure. In another example, a first sample can be obtained from a first bodily location or system of a subject (e.g., using a first collection technique) and a second sample can be obtained from a second bodily location or system of the subject (e.g., using a second collection technique), which second bodily location or system can be different than the first bodily location or system. In another example, multiple samples can be obtained from a subject at a same time from the same or different bodily locations. Different samples, such as different samples collected from different bodily locations of a same subject, at different times, from multiple different subjects, and/or using different collection techniques, can undergo the same or different processing (e.g., as described herein). For example, a first sample can undergo a first processing protocol and a second sample can undergo a second processing protocol.

A sample can be a biological sample, such as a cell sample (e.g., as described herein). A sample can include one or more biological particles, such as one or more cells and/or cellular constituents, such as one or more cell nuclei. For example, a sample can include a plurality of cells and/or cellular constituents. Components (e.g., cells or cellular constituents, such as cell nuclei) of a sample can be of a single type or a plurality of different types. For example, cells of a sample can include one or more different types of blood cells.

A biological sample can include a plurality of cells having different dimensions and features. In some cases, processing of the biological sample, such as cell separation and sorting (e.g., as described herein), can affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process can include the use of microfluidics (e.g., to separate biological particles of different sizes, types, charges, or other features).

In an example, a sample including one or more cells can be processed to separate the one or more cells from other materials in the sample (e.g., using centrifugation and/or another process). In some cases, cells and/or cellular constituents of a sample can be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process can include a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells can include, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method. For example, a flow cytometry method can be used to detect cells and/or cellular constituents based on a parameter such as a size, morphology, or protein expression. Flow cytometry-based cell sorting can include injecting a sample into a sheath fluid that conveys the cells and/or cellular constituents of the sample into a measurement region one at a time. In the measurement region, a light source such as a laser can interrogate the cells and/or cellular constituents and scattered light and/or fluorescence can be detected and converted into digital signals. A nozzle system (e.g., a vibrating nozzle system) can be used to generate droplets (e.g., aqueous droplets) including individual cells and/or cellular constituents. Droplets including cells and/or cellular constituents of interest (e.g., as determined via optical detection) can be labeled with an electric charge (e.g., using an electrical charging ring), which charge can be used to separate such droplets from droplets including other cells and/or cellular constituents. For example, FACS can include labeling cells and/or cellular constituents with fluorescent markers (e.g., using internal and/or external biomarkers). Cells and/or cellular constituents can then be measured and identified one by one and sorted based on the emitted fluorescence of the marker or absence thereof. MACS can use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS can use microbubbles (e.g., glass microbubbles) labeled with antibodies to target cells of interest. Cells and/or cellular components coupled to microbubbles can float to a surface of a solution, thereby separating target cells and/or cellular components from other components of a sample. Cell separation techniques can be used to enrich for populations of cells of interest (e.g., prior to partitioning, as described herein). For example, a sample including a plurality of cells including a plurality of cells of a given type can be subjected to a positive separation process. The plurality of cells of the given type can be labeled with a fluorescent marker (e.g., based on an expressed cell surface marker or another marker) and subjected to a FACS process to separate these cells from other cells of the plurality of cells. The selected cells can then be subjected to subsequent partition-based analysis (e.g., as described herein) or other downstream analysis. The fluorescent marker can be removed prior to such analysis or can be retained. The fluorescent marker can include an identifying feature, such as a nucleic acid barcode sequence and/or unique molecular identifier.

In another example, a first sample including a first plurality of cells including a first plurality of cells of a given type (e.g., immune cells expressing a particular marker or combination of markers) and a second sample including a second plurality of cells including a second plurality of cells of the given type can be subjected to a positive separation process. The first and second samples can be collected from the same or different subjects, at the same or different types, from the same or different bodily locations or systems, using the same or different collection techniques. For example, the first sample can be from a first subject and the second sample can be from a second subject different than the first subject. The first plurality of cells of the first sample can be provided a first plurality of fluorescent markers configured to label the first plurality of cells of the given type. The second plurality of cells of the second sample can be provided a second plurality of fluorescent markers configured to label the second plurality of cells of the given type. The first plurality of fluorescent markers can include a first identifying feature, such as a first barcode, while the second plurality of fluorescent markers can include a second identifying feature, such as a second barcode, that is different than the first identifying feature. The first plurality of fluorescent markers and the second plurality of fluorescent markers can fluoresce at the same intensities and over the same range of wavelengths upon excitation with a same excitation source (e.g., light source, such as a laser). The first and second samples can then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples can undergo separate FACS processes and the positively selected cells of the given type from the first sample and the positively selected cells of the given type from the second sample can then be combined for subsequent analysis. The encoded identifying features of the different fluorescent markers can be used to identify cells originating from the first sample and cells originating from the second sample. For example, the first and second identifying features can be configured to interact (e.g., in partitions, as described herein) with nucleic acid barcode molecules (e.g., as described herein) to generate barcoded nucleic acid products detectable using, e.g., nucleic acid sequencing.

### Multiplexing Methods

In some embodiments of the disclosure, steps (a) and (b) of the methods described herein are performed in multiplex format. For example, in some embodiments, step (a) of the methods disclosed herein can include individually partitioning additional single biological particles, such as nuclei, cell beads, cells, or engineered cells (e.g., engineered B cells) of the plurality of biological particles (e.g., nuclei, cell beads, cells, B cells) in additional partitions of the first plurality of partitions, and step (b) can further include determining all or a part of the nucleic acid sequences encoding antibodies or antigen-binding fragments thereof produced by the additional single biological particles, such as nuclei, cell beads, cells, or engineered cell (e.g., engineered B cells).

Accordingly, in some embodiments, the present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more biological particles (e.g., cells or nuclei) or features of biological particles (e.g., cells or nuclei) can be used to characterize biological particles (e.g., cells or nuclei) and/or cell/nucleus features. In some instances, cell features include cell surface features. Cell surface features can include, but are not limited to, a receptor, an antigen or antigen fragment, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof. A labelling agent can include, but is not limited to, a protein, a peptide, an antibody (or an epitope binding fragment thereof), an antigen, an antigen fragment, a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a Darpin, and a protein scaffold, or any combination thereof. The labelling agents can include (e.g., are attached to) a reporter oligonucleotide that is indicative of the cell surface feature to which the binding group binds. For example, the reporter oligonucleotide can include a barcode sequence that permits identification of the labelling agent. For example, a labelling agent that is specific to one type of cell feature (e.g., a first cell surface feature) can have a first reporter oligonucleotide coupled thereto, while a labelling agent that is specific to a different cell feature (e.g., a second cell surface feature) can have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969.

In a particular example, a library of potential cell feature labelling agents can be provided, where the respective cell feature labelling agents are associated with nucleic acid reporter molecules, such that a different reporter oligonucleotide sequence is associated with each labelling agent capable of binding to a specific cell feature. In other aspects, different members of the library can be characterized by the presence of a different oligonucleotide sequence label. For example, an antibody capable of binding to a first protein can have associated with it a first reporter oligonucleotide sequence, while an antibody, (which may be the same antibody), capable of binding to a second protein can have a different, (or additional if the same antibody), reporter oligonucleotide sequence(s) associated with it. The presence of the particular oligonucleotide sequence(s) can be indicative of the presence of a particular antibody or cell feature which can be recognized or bound by the particular antibody.

Labelling agents capable of binding to or otherwise coupling to one or more biological particles (e.g., cells or nuclei) can be used to characterize a biological particle (e.g., cell or nucleus) as belonging to a particular set of biological particles (e.g., cells or nuclei). For example, labeling agents can be used to label a sample of cells, e.g., to provide a sample index. For other example, labelling agents can be used to label a group of cells belonging to a particular experimental condition. In this way, a group of cells can be labeled as different from another group of cells. In an example, a first group of cells can originate from a first sample and a second group of cells can originate from a second sample. Labelling agents can allow the first group and second group to have a different labeling agent (or reporter oligonucleotide associated with the labeling agent). This can, for example, facilitate multiplexing, where cells of the first group and cells of the second group can be labeled separately and then pooled together for downstream analysis. The downstream detection of a label can indicate analytes as belonging to a particular group.

For example, a reporter oligonucleotide can be linked to an antibody or an epitope binding fragment thereof, and labeling a cell can include subjecting the antibody-linked barcode molecule or the epitope binding fragment-linked barcode molecule to conditions suitable for binding the antibody to a molecule present on a surface of the cell. The binding affinity between the antibody or the epitope binding fragment thereof and the molecule present on the surface can be within a desired range to ensure that the antibody or the epitope binding fragment thereof remains bound to the molecule. For example, the binding affinity can be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds can be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 M, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant can be less than about 10 pM. In some embodiments, the antibody or antigen-binding fragment thereof has a desired dissociation rate constant (koff), such that the antibody or antigen binding fragment thereof remains bound to the target antigen or antigen fragment during various sample processing steps

In another example, a reporter oligonucleotide can be coupled to a cell-penetrating peptide (CPP), and labeling cells can include delivering the CPP coupled reporter oligonucleotide into a biological particle. Labeling biological particles can include delivering the CPP conjugated oligonucleotide into a cell and/or cell bead by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide. In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells can include subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649 for a description of organic fluorophores.

A reporter oligonucleotide can be coupled to a lipophilic molecule, and labeling cells can include delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane can be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide can enter into the intracellular space and/or a cell nucleus. In some embodiments, a reporter oligonucleotide coupled to a lipophilic molecule will remain associated with and/or inserted into lipid membrane (as described herein) via the lipophilic molecule until lysis of the cell occurs, e.g., inside a partition. Exemplary embodiments of lipophilic molecules coupled to reporter oligonucleotides are described in PCT/US2018/064600.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to partitioning, the biological particles (e.g., cells or nuclei) can be incubated with the library of labelling agents, that can be labelling agents to a broad panel of different biological particle features, such as cell or nuclear features, e.g., receptors, proteins, etc., and which include their associated reporter oligonucleotides. Unbound labelling agents can be washed from the biological particles (e.g., cells or nuclei), and the biological particles (e.g., cells or nuclei) can then be co-partitioned (e.g., into droplets or wells) along with partition-specific barcode oligonucleotides (e.g., attached to a support, such as a bead or gel bead) as described elsewhere herein. As a result, the partitions can include the biological particle(s) (e.g., cell/nucleus or cells/nuclei), as well as the bound labelling agents and their known, associated reporter oligonucleotides.

In other instances, e.g., to facilitate sample multiplexing, a labelling agent that is specific to a particular biological particle (e.g., cell or nucleus) feature can have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent can interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088.

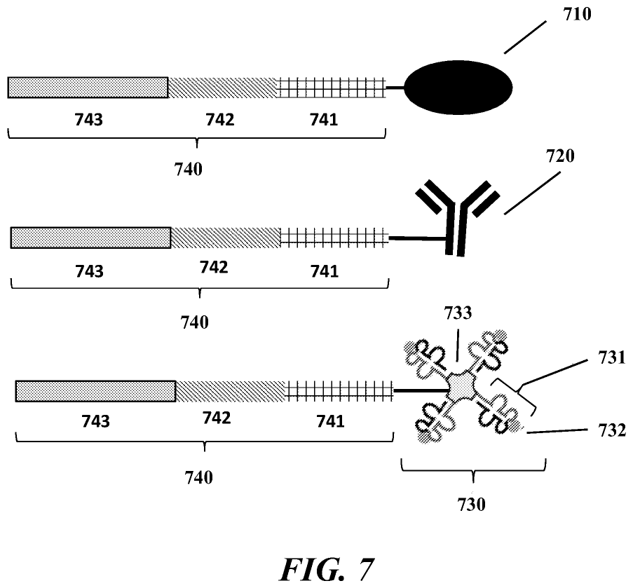

In some embodiments, to facilitate sample multiplexing, individual samples can be stained with lipid tags, such as cholesterol-modified oligonucleotides (CMOs, see, e.g., FIG. 7), anti-calcium channel antibodies, or anti-ACTB antibodies. Non-limiting examples of anti-calcium channel antibodies include anti-KCNN4 antibodies, anti-BK channel beta 3 antibodies, anti-a1B calcium channel antibodies, and anti-CACNA1A antibodies. Examples of anti-ACTB antibodies suitable for the methods of the disclosure include, but are not limited to, mAbGEa, ACTN05, AC-15, 15G5A11/E2, BA3R, and HHF35.

As described elsewhere herein, libraries of labelling agents can be associated with a particular biological particle (e.g., cell or nucleus) feature as well as be used to identify analytes as originating from a particular biological particle (e.g., cell or nucleus) population, or sample. Cell populations can be incubated with a plurality of libraries such that a cell or cells include multiple labelling agents. For example, a cell/nucleus can include coupled thereto a lipophilic labeling agent and an antibody. The lipophilic labeling agent can indicate that the cell/nucleus is a member of a particular cell/nucleus sample, whereas the antibody can indicate that the cell/nucleus includes a particular analyte. In this manner, the reporter oligonucleotides and labelling agents can allow multi-analyte, multiplexed analyses to be performed.

In some instances, these reporter oligonucleotides can include nucleic acid barcode sequences that permit identification of the labelling agent which the reporter oligonucleotide is coupled to. The use of oligonucleotides as the reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents can be achieved through any of a variety of direct or indirect, covalent or non-covalent associations or attachments. For example, oligonucleotides can be covalently attached to a portion of a labelling agent (such a protein, e.g., an antigen or antigen fragment, an antibody or antibody fragment) using chemical conjugation techniques (e.g., Lightning-Link® antibody labelling kits available from Innova Biosciences), as well as other non-covalent attachment mechanisms, e.g., using biotinylated antibodies (or biotinylated antigens, or biotinylated antigen fragments) and oligonucleotides (or beads that include one or more biotinylated linker, coupled to oligonucleotides) with an avidin, and streptavidin linker in monomeric or multimeric form (e.g., tetramic form of streptavidin). Those of skill in the art will recognize that a streptavidin monomer encompasses streptavidin molecules with 1 biotin binding site, while a streptavidin multimer encompasses strepatavidin molecules with more than 1 biotin binding site. For example, a streptavidin tetramer has 4 biotin binding sites. However, a skilled artisan will also recognize that in a streptavidin tetramer does not necessarily 4 streptavidins complexed together. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715. Likewise, protein and peptide biotinylation techniques have been developed and are readily available. See, e.g., U.S. Pat. No. 6,265,552. Furthermore, click reaction chemistry such as a Methyltetrazine-PEG5-NHS Ester reaction, a TCO-PEG4-NHS Ester reaction, or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the label agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent (e.g., an antigen, an antigen fragment, an antibody, an antibody fragment) is presented as a monomer. In some cases, the labelling agent is presented as a multimer. In some cases, a labelling agent (e.g., an antigen, an antigen fragment, an antibody, an antibody fragment) is presented as a dimer. In some cases, a labelling agent (e.g., an antigen, an antigen fragment, an antibody, an antibody fragment) is presented as a trimer. In some cases, a labelling agent (e.g., an antigen, an antigen fragment, an antibody, an antibody fragment) is presented as a tetramer. In some cases, a labelling agent (e.g., an antigen, an antigen fragment, an antibody, an antibody fragment) is presented as a pentamer. In some cases, a labelling agent (e.g., an antigen, an antigen fragment, an antibody, an antibody fragment) is presented as a hexamer. In some cases, a labelling agent (e.g., an antigen, an antigen fragment, an antibody, an antibody fragment) is presented as a heptamer. In some cases, a labelling agent (e.g., an antigen, an antigen fragment, an antibody, an antibody fragment) is presented as an octamer. In some cases, a labelling agent (e.g., an antigen, an antigen fragment, an antibody, an antibody fragment) is presented as a nonamer. In some cases, a labelling agent (e.g., an antigen, an antigen fragment, an antibody, an antibody fragment) is presented as a decamer. In some cases, a labelling agent (e.g., an antigen, an antigen fragment, an antibody, an antibody fragment) is presented as a 10+−mer.

In some cases, the labelling agent can include a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide can be allowed to hybridize to the reporter oligonucleotide.

FIG. 7 describes exemplary labelling agents (710, 720, 730) including reporter oligonucleotides (740) attached thereto. Labelling agent 710 (e.g., any of the labelling agents described herein) is attached (either directly, e.g., covalently attached, or indirectly) to reporter oligonucleotide 740. Reporter oligonucleotide 740 can include barcode sequence 742 that identifies labelling agent 710. Reporter oligonucleotide 740 can also include one or more functional sequences 743 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 7, in some instances, reporter oligonucleotide 740 conjugated to a labelling agent (e.g., 710, 720, 730) includes a functional sequence 741, a reporter barcode sequence 742 that identifies the labelling agent (e.g., 710, 720, 730), and reporter capture handle 743. Reporter capture handle sequence 743 can be configured to hybridize to a complementary sequence, such as a complementary sequence present on a nucleic acid barcode molecule 790 (not shown), such as those described elsewhere herein. In some instances, nucleic acid barcode molecule 790 is attached to a support (e.g., a bead, such as a gel bead), such as those described elsewhere herein. For example, nucleic acid barcode molecule 790 can be attached to the support via a releasable linkage (e.g., including a labile bond), such as those described elsewhere herein. In some instances, reporter oligonucleotide 740 includes one or more additional functional sequences, such as those described above.

In some instances, the labelling agent 710 is a protein or polypeptide (e.g., an antigen or prospective antigen, or a fragment of an antigen or prospective antigen) including reporter oligonucleotide 740. Reporter oligonucleotide 740 includes reporter barcode sequence 742 that identifies polypeptide 710 and can be used to infer the presence of an analyte, e.g., a binding partner of polypeptide 710 (i.e., a molecule or compound to which polypeptide 710 can bind). In some instances, the labelling agent 710 is a lipophilic moiety (e.g., cholesterol) including reporter oligonucleotide 740, where the lipophilic moiety is selected such that labelling agent 710 integrates into a membrane of a cell or nucleus. Reporter oligonucleotide 740 includes reporter barcode sequence 742 that identifies lipophilic moiety 710 which in some instances is used to tag biological particles such as cells or nuclei (e.g., groups of cells or nuclei, cell samples, etc.) and can be used for multiplex analyses as described elsewhere herein. In some instances, the labelling agent is an antibody 720 (or an epitope binding fragment thereof) including reporter oligonucleotide 740. Reporter oligonucleotide 740 includes reporter barcode sequence 742 that identifies antibody 720 and can be used to infer the presence of, e.g., a target of antibody 720 (i.e., a molecule or compound to which antibody 720 binds). In other embodiments, labelling agent 730 includes an MHC molecule 731 including peptide 732 and reporter oligonucleotide 740 that identifies peptide 732. In some instances, the MHC molecule is coupled to a support 733. In some instances, support 733 can be a polypeptide, such as streptavidin, or a polysaccharide, such as dextran. In some instances, reporter oligonucleotide 740 can be directly or indirectly coupled to MHC labelling agent 730 in any suitable manner. For example, reporter oligonucleotide 740 can be coupled to MHC molecule 731, support 733, or peptide 732. In some embodiments, labelling agent 730 includes a plurality of MHC molecules, (e.g. is an MHC multimer, which can be coupled to a support (e.g., 733)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5@ MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969.

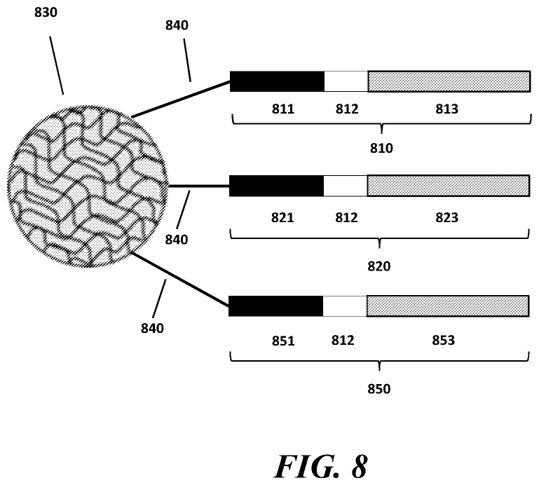

Exemplary barcode molecules attached to a support (e.g., a bead) is shown in FIG. 8. In some embodiments, analysis of multiple analytes (e.g., RNA and one or more analytes using labelling agents described herein) can include nucleic acid barcode molecules as generally depicted in FIG. 8. In some embodiments, nucleic acid barcode molecules 810 and 820 are attached to support 830 via a releasable linkage 840 (e.g., including a labile bond) as described elsewhere herein. Nucleic acid barcode molecule 810 can include functional sequence 811, barcode sequence 812 and capture sequence 813. Nucleic acid barcode molecule 820 can include adapter sequence 821, barcode sequence 812, and capture sequence 823, wherein capture sequence 823 includes a different sequence than capture sequence 813. In some instances, adapter 811 and adapter 821 include the same sequence. In some instances, adapter 811 and adapter 821 include different sequences. Although support 830 is shown including nucleic acid barcode molecules 810 and 820, any suitable number of barcode molecules including common barcode sequence 812 are contemplated herein. For example, in some embodiments, support 830 further includes nucleic acid barcode molecule 850. Nucleic acid barcode molecule 850 can include adapter sequence 851, barcode sequence 812 and capture sequence 853, wherein capture sequence 853 includes a different sequence than capture sequence 813 and 823. In some instances, nucleic acid barcode molecules (e.g., 810, 820, 850) include one or more additional functional sequences, such as a UMI or other sequences described herein. The nucleic acid barcode molecules 810, 820 or 850 can interact with analytes as described elsewhere herein, for example, as depicted in FIGS. 9A-9C.

Referring to FIG. 9A, in an instance where biological particles (e.g., cells or nuclei) are labelled with labeling agents, capture sequence 923 can be complementary to an adapter sequence of a reporter oligonucleotide. Biological particles (e.g., cells or nuclei) can be contacted with one or more reporter oligonucleotide 920 conjugated labelling agents 910 (e.g., polypeptide such as an antigen or fragment of an antigen, antibody, or others described elsewhere herein). In some cases, the biological particles (e.g., cells or nuclei) can be further processed prior to barcoding. For example, such processing steps can include one or more washing and/or cell sorting steps. In some instances, a biological particle (e.g., cell or nucleus) that is bound to labelling agent 910 which is conjugated to reporter oligonucleotide 920 and support 930 (e.g., a bead, such as a gel bead) including nucleic acid barcode molecule 990 is partitioned into a partition amongst a plurality of partitions (e.g., a droplet of a droplet emulsion or a well of a microwell array). In some instances, the partition includes at most a single cell or nucleus bound to labelling agent 910. In some instances, reporter oligonucleotide 920 conjugated to labelling agent 910 (e.g., polypeptide such as an antigen or fragment of an antigen, an antibody, pMHC molecule such as an MHC multimer, etc.) includes a first functional sequence 911 (e.g., a primer sequence), a barcode sequence 912 that identifies the labelling agent 910 (e.g., the polypeptide such as an antigen or fragment of an antigen, antibody, or peptide of a pMHC molecule or complex), and a capture handle sequence 913. Capture handle sequence 913 can be configured to hybridize to a complementary sequence, such as capture sequence 923 present on a nucleic acid barcode molecule 990 (e.g., partition-specific barcode molecule). In some instances, oligonucleotide 910 includes one or more additional functional sequences, such as those described elsewhere herein.

Barcoded nucleic acid molecules can be generated (e.g., via a nucleic acid reaction, such as nucleic acid extension, reverse transcription, or ligation) from the constructs described in FIGS. 9A-9C. For example, capture handle sequence 913 can then be hybridized to complementary capture sequence 923 to generate (e.g., via a nucleic acid reaction, such as nucleic acid extension or ligation) a barcoded nucleic acid molecule including cell barcode (e.g., common barcode or partition-specific barcode) sequence 922 (or a reverse complement thereof) and reporter barcode sequence 912 (or a reverse complement thereof). In some embodiments, the nucleic acid barcode molecule 990 (e.g., partition-specific barcode molecule) further includes a UMI (925). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) can be performed. For example, the workflow can include a workflow as generally depicted in any of FIGS. 9A-9C, or a combination of workflows for an individual analyte, as described elsewhere herein. For example, by using a combination of the workflows as generally depicted in FIGS. 9A-9C, multiple analytes can be analyzed.

In some instances, analysis of an analyte (e.g. a nucleic acid, a polypeptide, a carbohydrate, a lipid, etc.) includes a workflow as generally depicted in FIG. 9A. A nucleic acid barcode molecule 990 can be co-partitioned with the one or more analytes. In some instances, nucleic acid barcode molecule 990 is attached to a support 930 (e.g., a bead, such as a gel bead), such as those described elsewhere herein. For example, nucleic acid barcode molecule 990 can be attached to support 930 via a releasable linkage 940 (e.g., including a labile bond), such as those described elsewhere herein. Nucleic acid barcode molecule 990 can include a functional sequence 921 and optionally include other additional sequences, for example, a barcode sequence 922 (e.g., common barcode, partition-specific barcode, or other functional sequences described elsewhere herein), and/or a UMI sequence 925. The nucleic acid barcode molecule 990 can include a capture sequence 923 that can be complementary to another nucleic acid sequence, such that it can hybridize to a particular sequence.

For example, capture sequence 923 can include a poly-T sequence and can be used to hybridize to mRNA. Referring to FIG. 9C, in some embodiments, nucleic acid barcode molecule 990 includes capture sequence 923 complementary to a sequence of RNA molecule 960 from a cell. In some instances, capture sequence 923 includes a sequence specific for an RNA molecule. Capture sequence 923 can include a known or targeted sequence or a random sequence. In some instances, a nucleic acid extension reaction can be performed, thereby generating a barcoded nucleic acid product including capture sequence 923, the functional sequence 921, UMI sequence 925, any other functional sequence, and a sequence corresponding to the RNA molecule 960.

In another example, capture sequence 923 can be complementary to an overhang sequence or an adapter sequence that has been appended to an analyte. For example, referring to FIG. 9B, in some embodiments, primer 950 includes a sequence complementary to a sequence of nucleic acid molecule 960 (such as an RNA encoding for a BCR sequence) from a biological particle. In some instances, primer 950 includes one or more sequences 951 that are not complementary to RNA molecule 960. Sequence 951 can be a functional sequence as described elsewhere herein, for example, an adapter sequence, a sequencing primer sequence, or a sequence the facilitates coupling to a flow cell of a sequencer. In some instances, primer 950 includes a poly-T sequence. In some instances, primer 950 includes a sequence complementary to a target sequence in an RNA molecule. In some instances, primer 950 includes a sequence complementary to a region of an immune molecule, such as the constant region of a BCR sequence. Primer 950 is hybridized to nucleic acid molecule 960 and complementary molecule 970 is generated. For example, complementary molecule 970 can be cDNA generated in a reverse transcription reaction. In some instances, an additional sequence can be appended to complementary molecule 970. For example, the reverse transcriptase enzyme can be selected such that several non-templated bases 980 (e.g., a poly-C sequence) are appended to the cDNA. In another example, a terminal transferase can also be used to append the additional sequence. Nucleic acid barcode molecule 990 includes a sequence 924 complementary to the non-templated bases, and the reverse transcriptase performs a template switching reaction onto nucleic acid barcode molecule 990 to generate a barcoded nucleic acid molecule including cell (e.g., partition specific) barcode sequence 922 (or a reverse complement thereof) and a sequence of complementary molecule 970 (or a portion thereof). In some instances, capture sequence 923 includes a sequence complementary to a region of an immune molecule, such as the constant region of a BCR sequence. Capture sequence 923 is hybridized to nucleic acid molecule 960 and a complementary molecule 970 is generated. For example, complementary molecule 970 can be generated in a reverse transcription reaction generating a barcoded nucleic acid molecule including cell barcode (e.g., common barcode or partition-specific barcode) sequence 922 (or a reverse complement thereof) and a sequence of complementary molecule 970 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969.

In some embodiments, biological particles (e.g., cells or nuclei) from a plurality of samples (e.g., a plurality of subjects) can be pooled, sequenced, and demultiplexed by identifying mutational profiles associated with individual samples and mapping sequence data from single biological particles to their source based on their mutational profile. See, e.g., Xu J. et al., Genome Biology Vol. 20, 290 (2019); Huang Y. et al., Genome Biology Vol. 20, 273 (2019); and Heaton et al., Nature Methods volume 17, pages 615-620(2020), which are hereby incorporated by reference in their entireties.

### Combinatorial Barcoding

In some instances, barcoding of a nucleic acid molecule may be done using a combinatorial approach. In such instances, one or more nucleic acid molecules (which may be comprised in a cell or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating first barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The first barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the first barcoded nucleic acid molecules, thereby generating second barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The second barcoded nucleic acid molecules may thus comprise two barcode sequences (e.g., from the first nucleic acid barcode molecules and the second nucleic acid barcode molecules).

Additional barcode sequences may be attached to the second barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 5, 6, 7, 8, 9, 10 or more operations of splitting (e.g., partitioning) and/or pooling (e.g., from the partitions). Additional examples of combinatorial barcoding may also be found in International Patent Publication Nos. WO2019/165318, each of which is herein entirely incorporated by reference for all purposes.

Beneficially, the combinatorial barcode approach may be useful for generating greater barcode diversity, and synthesizing unique barcode sequences on nucleic acid molecules derived from a cell or partition. For example, combinatorial barcoding comprising three operations, each with 100 partitions, may yield up to 10⁶ unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g., a sample or target nucleic acid molecule) in successive operations within a partition (e.g., droplet or well) to generate a first barcoded nucleic acid molecule. A second unique nucleic acid molecule comprising one or more nucleic acid bases may be attached to the first barcoded nucleic acid molecule molecule, thereby generating a second barcoded nucleic acid molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in PCT/US2018/067356 and U.S. Pat. Pub. No. 2019/0330694, which are hereby incorporated by reference in its entirety.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

### Example 1: Method of Analyzing Binding Affinity of Antigen-Binding Molecules

This Example provides a general procedure using stgRNA tracking to identify an antigen-binding molecule with desired affinity.

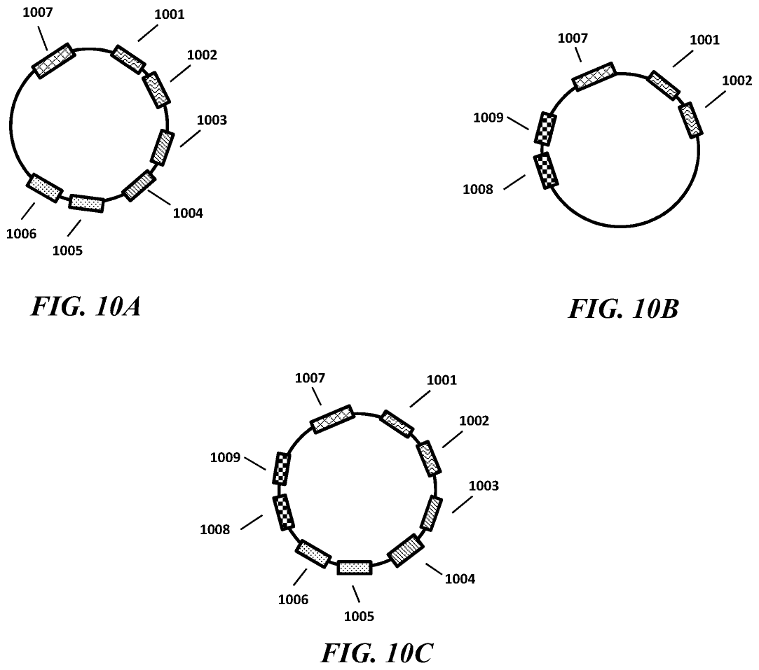

In this Example, cells are engineered to express the construct as illustrated in FIG. 10A. Briefly, the construct comprises a first promoter 1001 operably linked to nucleic acid sequence encoding an antigen-binding molecule 1002 (e.g., a BCR), a second promoter 1003 operably linked to nucleic acid sequence encoding a stgRNA 1004, and a NFkB promoter 1005 operably linked to a coding sequence for Cas protein 1006 (e.g., Cas9). In some embodiments, the construct does not comprise first promoter 1001 operably linked to antigen-binding molecule 1002. For example, in cases wherein the cells are B cells that are known to express an antigen-binding molecule sequence of interest, the construct does not comprise first promoter 1001 or nucleic acid sequence encoding antigen-binding molecule 1002. In some experiments, an A1 or c-Myc promoter can be used in place of the NFkB promoter 1005. The BCR sequence 1002 can be modified and expressed in various ways. In some cases, the second promoter 1003 can be a constitutive promoter capable of driving constitutive expression of a coding sequence operably linked downstream thereof, or an inducible promoter capable of driving expression dependent on a response element, such as a nutritional response element. In some experiments, the construct is engineered to also include one or more selection markers 1007. The one or more selection markers 1007 can be any suitable markers, such as, GFP or drug resistance.

Cells are engineered to express the construct, e.g., by transduction or transfection. Cells, such as B cells, can be transduced or transfected by any suitable methods in the art.

Subsequently, the engineered (e.g., transduced) cells are incubated with an antigen of interest. In addition, the transduced cells can also be incubated with one or more co-stimulatory molecules, such as BAFF, CD40-L, anti-c, and anti-k. Furthermore, the transduced cells can also be a cell feeder layer for B cells, or anti-CD28 or anti-4-1BB for T cells. BCR-expressing cells that bind the antigen trigger NFkB expression of Cas. Cas induces stgRNA to self-target and mutate. In these experiments, the number of mutations within the stgRNA sequence induced by the NFkB-driven Cas protein is a readout of BCR-mediated immune response by the antigen, with higher number of mutations indicating greater antigen binding.

The cells are subsequently partitioned into the discrete droplets for barcoding according to methods disclosed herein. Accordingly, components from each cell carry a partition-specific barcode that allows each of the component, such as the stgRNA and BCR sequences to be identified by methods described in the present disclosure, such as high-throughput sequencing (e.g., NGS). Thus, the stgRNA sequences are determined. As stated above, the number of mutations induced in the stgRNA indicate NFkB driven Cas immune response by the antigen binding to the BCR, with higher number of mutations indicating greater antigen binding. Subsequently, the BCR of interest which have increased affinity to the specific antigen, as indicated by an increased number of mutations, are selected and optionally isolated for further analysis.

### Example 2: Method for Affinity-Maturing an Antigen-Binding Molecule

In this Example, the cells are engineered (e.g., transduced or transfected) with a construct as illustrated in FIG. 10B. Briefly, the construct used in this experiment does not include the promoter 1003 and stgRNA 1004. Further, the construct used in this experiment does not include promoter 1005 and Cas 1006. Instead, the construct contains a coding sequence for an SHM enzyme (such as an activation-induced cytidine deaminase (AID)) 1009 operably linked to an inducible promoter (e.g., an NFkB promoter) 1008. In addition, the construct contains a coding sequence for an antigen-binding molecule of interest 1002 (e.g., an antibody, or a BCR) operably linked to promoter 1001.

The engineered (e.g., transduced or transfected) cells are then incubated with an antigen of interest. In these experiments, antigen binding to the antigen-binding molecule of interest 1002 triggers NFkB expression of the SHM enzyme 1009 which induces somatic hypermutation into the antigen-binding molecule coding sequence 1002. Thus, antigen-binding molecules that bind to the target antigen selectively undergo SHM and affinity maturation. Subsequently, the engineered (e.g., transduced or tranfected) cells are partitioned into the discrete droplets and processed as in Example 1. The antibody of interest is identified, selected, and isolated.

### Example 3: Use of STGRNA Mutation Tracking and SHM

In this Example, both stgRNA mutation tracking and SHM are used in parallel to analyze and affinity mature an antigen-binding molecule.

In this Example, cells are engineereed (e.g., transduced or tranfected) with the construct illustrated in FIG. 10C. The construct contains (i) a first promoter 1001 operably linked to a coding sequence for an antigen-binding molecule 1002 (e.g., a BCR), (ii) a second promoter 1003 operably linked to a coding sequence for a stgRNA 1004, (iii) a NFkB promoter 1005 operably linked to a coding sequence for Cas 1006, and (iv) a coding sequence for an SHM enzyme 1009 (such as AID) operably linked to another inducible promoter 1008 (e.g., an NFkB promoter). The engineered (e.g., transduced or tranfected) cells are then incubated with an antigen of interest. Antigen binding to the cells triggers the expression of promoters 1005 and 1008, which in turn drive expression of Cas and SHM, respectively. The SHM 1009 introduces antigen-specific mutations into the BCR coding sequence 1002 in a dose-dependent fashion. Similarly, the stgRNA is self-edited in a binding activity-dependent fashion. Subsequently, the transduced cells are partitioned into the discrete droplets and processed as in Example 1. Sequencing of the mutated BCR using any methods known in the art or described herein enables the readout of mutations from the SHM enzyme 1009. In addition, sequencing of the stgRNA provides parallel confirmation.

### Example 4: Error Prone Amplification

This Example provides a general procedure for performing error-prone amplification to generate variant antibodies, and variant antigen-binding fragments thereof, that may be identified as having an altered characteristic based on stgRNA mutations.

Error prone PCR is performed on purified antibody clone material, e.g., comprising a selected antibody or selected antigen-binding fragment thereof, which may be from a construct (or a set of constructs) that include the coding sequence for the antibody or an antigen-binding fragment of the antibody or may be from a cDNA library. By amplifying the target sequences using Taq DNA polymerase in the presence of Mn, Mg, and K salts, with variable ratios of dNTPs, pools of product containing random mutations are created. The high divalent cation environment of the error-prone PCR reaction leads to an increase in the frequency of improper base pairing events. The products accordingly comprise nucleic acid sequences encoding variant antibodies or antigen-binding fragments.

These products are inserted into expression vectors and transfected into cells, e.g., B cells. In addition, a sequence encoding the selected antibody or selected antigen-binding fragment thereof is inserted into an expression vector and transfected into cells, e.g., B cells. The cells are then screened for characteristics including antibody specificity and/or antibody affinity.

In some instances, the products are inserted into an expression construct according to the methods described herein, e.g., described in Example 1. In addition, in such instances, a sequence or sequences encoding the selected antibody or selected antigen-binding fragment thereof is inserted into an expression construct according to the method described in Example 1.

Cells are engineered to express the construct, e.g., via transduction or transfection.

Subsequently, the transduced cells are incubated with an antigen of interest. In addition, the transduced cells can also be incubated with one or more co-stimulatory molecules, such as BAFF, CD40-L, anti-c, and anti-k. Furthermore, the transduced cells can also be a cell feeder layer for B cells, or anti-CD28 or anti-4-1BB for T cells. BCR-expressing cells that bind the antigen trigger NFkB expression of Cas. Cas induces stgRNA to self-target and mutate. In these experiments, the number of mutations within the stgRNA sequence induced by the NFkB-driven Cas protein is a readout of BCR-mediated immune response by the antigen, with higher number of mutations indicating greater antigen binding.

The cells are subsequently partitioned into the discrete droplets for barcoding according to methods disclosed herein. Accordingly, components from each cell carry a partition-specific barcode that allows each of the component, such as the stgRNA and BCR sequences to be identified by methods described in the present disclosure, such as high-throughput sequencing (e.g., NGS). Thus, the stgRNA sequences are determined. As stated above, the number of mutations induced in the stgRNA indicate NFkB driven Cas immune response by the antigen binding to the BCR, with higher number of mutations indicating greater antigen binding. Subsequently, the BCR of interest which have increased affinity to the specific antigen, as indicated by an increased number of mutations, are selected and optionally isolated for further analysis.

In some instances, the products are inserted into an expression construct according to a method described in Example 3. In some instances, a sequence or sequences encoding the selected antibody or selected antigen-binding fragment thereof is inserted into an expression construct according to the method described in Example 3.

Cells are engineered to express the construct, e.g., via transduction or transfection. Reference engineered cells may also be provided. The engineered cells are then incubated with an antigen of interest. Antigen binding to the cells triggers the expression of promoters 1005 and 1008, which in turn drive expression of Cas and SHM, respectively. The SHM 1009 introduces antigen-specific mutations into the BCR coding sequence 1002 in a dose-dependent fashion. Similarly, the stgRNA is self-edited in a binding activity-dependent fashion. Subsequently, the transduced cells are partitioned into the discrete droplets and processed as in Example 1. Sequencing of the mutated BCR using any methods known in the art or described herein enables the readout of mutations from the SHM enzyme 1009. In addition, sequencing of the stgRNA provides parallel confirmation.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1. An nucleic acid construct comprising:
a) a third nucleic acid sequence encoding an antigen-binding molecule of interest, and
b) a nucleic acid sequence encoding an enzyme capable of mediating SHM, wherein the coding sequence for the SHM enzyme is operably linked to an inducible promoter, wherein the inducible promoter is induced by an immune response.

2. The nucleic acid construct of claim 1, wherein the inducible promoter is selected from the group consisting of a nucleotide factor kappa-B (NFkB) promoter, a MyD88 promoter, an interleukin (IL)-2 promoter, and a JAK/STAT promoter, a Bcl-xL promoter, a c-Rel promoter, a Myc promoter, a NFAT promoter, a mTORC1 promoter, a CD40 promoter, a TLR9 promoter, a Btk promoter, a PI3K promoter, an Arp2/3 promoter, and an IL-4 promoter.

3. The nucleic acid construct of claim 1, wherein the SHM enzyme is selected from the group consisting of RAG1, RAG2, APOBEC3F, activation-induced cytidine deaminase (AID), POLH/, Pol η, Pol θ, Polι, Polζ, Pol λ, REV1, PCNA, UNG, and a functional mutant thereof.

4. The nucleic acid construct of claim 1, wherein the third nucleic acid sequence is heterologous or non-heterologous.

5. The nucleic acid construct of claim 1, wherein the third nucleic acid sequence is operably linked to a promoter.

6. (canceled)

7. A nucleic acid construct comprising one or more of the following:
a) a first heterologous nucleic acid sequence encoding a self-targeting guide RNA (stgRNA) operably linked to a first promoter,
b) a second heterologous nucleic acid sequence encoding a CRISPR-associated (Cas) protein operably linked to a second promoter, wherein the second promoter is induced by an immune response; and
c) a third nucleic acid sequence encoding an antigen-binding molecule of interest.

8-27. (canceled)

28. The nucleic acid construct of claim 1, wherein the antigen-binding molecule comprises an immune receptor, an immunoglobulin, and an antibody or a functional fragment thereof.

29. The nucleic acid construct of claim 28, wherein the antigen-binding molecule is an immunoglobulin.

30. The nucleic acid construct of claim 29, wherein the immunoglobulin is selected from the group consisting of IgA, IgD, IgE, IgG, and IgM.

31. The nucleic acid construct of claim 28, wherein the antigen-binding molecule is an antibody or a functional fragment thereof.

32-33. (canceled)

34. The nucleic acid construct of claim 28, wherein the immune receptor comprises a B cell receptor, a chimeric antigen receptor, a membrane-bound antibody, and a membrane-bound immunoglobulin superfamily member.

35-36. (canceled)

37. The nucleic acid construct of claim 28, wherein the third nucleic acid sequence encoding the antigen-binding molecule is configured as a single chain comprising a first segment encoding a first variable region and a second segment encoding a second variable region.

38. The nucleic acid construct of claim 37, wherein the third nucleic acid sequence comprises a linker that operably links the first segment and the second segment.

39-42. (canceled)

43. A composition comprising a plurality of the nucleic acid constructs of claim 1.

44. A vector comprising the nucleic acid construct of claim 1.

45-46. (canceled)

47. A composition comprising a plurality of the vectors of claim 44.

48. (canceled)

49. An engineered cell comprising the nucleic acid construct of claim 1.

50-63. (canceled)

64. A method for affinity maturing an antigen-binding molecule of interest, comprising contacting the engineered cells of claim 49 with a target antigen of the antigen-binding molecule of interest.

65-95. (canceled)

96. A method of making an antigen-binding molecule of interest, comprising:
a) identifying the antigen-binding molecule of interest by the method of claim 64, and
b) purifying and/or producing the antigen-binding molecule from the identified single engineered cell expressing the antigen-binding molecule thereof.

97. An antigen-binding molecule identified by the method of claim 64.

98-99. (canceled)
